(19) United States
(12) Patent Application Publication
(10) Pub. No.: US 2024/0018526 A1
(43) Pub. Date: Jan. 18, 2024

(54) DEOXYRIBONUCLEIC ACID-BASED BIOSENSOR AND ASSOCIATED METHODS

(71) Applicant: FACible BioDiagnostics LLC, Meridian, ID (US)

(72) Inventors: Steven J. Burden, Boise, ID (US); Clémentine FN Gibard Bohachek, Boise, ID (US); Jonathon C. Reeck, Boise, ID (US); Hunter J. Covert, Boise, ID (US); Tanner B. Pollock, Boise, ID (US); Nicholas H. Shults, Boise, ID (US); Shiwei Li, Lower Hutt City (NZ); Aidan M. Poe, Boise, ID (US)

(21) Appl. No.: 18/005,590

(22) PCT Filed: Jul. 15, 2021
(86) PCT No.: PCT/US21/41883

§ 371 Date: Jan. 13, 2023

Related U.S. Application Data
(60) Provisional application No. 63/134,164, filed on Jan. 5, 2021.
(60) Provisional application No. 63/087,093, filed on Oct. 2, 2020.
(60) Provisional application No. 63/052,353, filed on Jul. 15, 2020.

(51) Int. Cl. C12N 15/115; G01N 33/569; G01N 33/533

(52) U.S. Cl. CPC C12N 15/115; G01N 33/56983; G01N 2333/165; C12N 2310/16; G01N 33/533

(57) ABSTRACT

The present disclosure relates to biosensors comprising a sensor region, a linker region, and a reporter region. The sensor region is a DNA aptamer and includes a target domain configured to bind to a target and a reporter domain configured to bind to a reporter. The linker domain operably connects the target domain to the reporter domain. Binding of the target to the target domain results in a conformational change, such as an allosteric change, to the aptamer resulting in second signal emitted by the reporter that differs from a first signal emitted by the reporter compared to the target unbound state. Methods of selecting biosensors and their use to detect the presence of a target in a sample are provided herein.

### CROSS-REFERENCE TO RELATED APPLICATIONS

This application claims the benefit of and priority to U.S. provisional patent application Ser. No. 63/052,353, filed Jul. 15, 2020; 63/087,093, filed Oct. 2, 2020; and 63/134,164, filed Jan. 5, 2021, which are each incorporated herein by reference in their entireties.

### TECHNICAL FIELD

The present application relates generally to nucleic acid-based biosensors comprising an aptamer (e.g., a DNA aptamer) and a reporter, and to methods of preparing and using the same. The present disclosure relates to the fields of biology, chemistry, medicinal chemistry, medicine, molecular biology, and pharmacology.

### BACKGROUND

Ribonucleic acid (RNA) aptamers are short, single-stranded RNA molecules that fold into stable three-dimensional shapes and are useful for binding to certain structural features of target molecules. RNA aptamers having high affinity and specificity for target molecules, such as proteins, nucleic acids, small molecules, and ions, have previously been selected from complex libraries using the Selective Enrichment of Ligands by Exponential Enrichment (SELEX) protocol (Tuerk and Gold, 1990; Ellington and Szostak, 1992, Ellington et al., 1990, In vitro selection of RNA molecules that bind specific ligands. Nature 346: 818-822. https://doi.org/10.1038/346818a0; Bock et al. 1992, Selection of single-stranded DNA molecules that bind and inhibit human thrombin. Nature 355: 564-566. https://doi.org/10.1038/355564a0, both herein incorporated in their entirety). Most therapeutic RNA aptamers are exogenously administered to cells that express a target molecule (e.g., a target cell) by binding to extracellular domains of certain cell surface proteins. These have been used to inhibit a function of the target molecule or as vehicles to deliver a therapeutic agent to the target cell. (Zhou, et al., 2009; Famalingam, et al., 2011; Ditzler, et al., 2011; Whatley, et al., 2013; Shum, Zhou and Rossi, 2013; Duclair, et al). Aptamers have also been constructed using deoxyribonucleic acid (DNA) using protocols that are generally the same, or similar to, SELEX while being adapted for DNA instead of RNA. These aptamers are referred to as DNA aptamers and generally throughout the application as aptamers.

In addition to therapeutic uses, aptamers can be designed to bind a reporter molecule, such as a fluorescent molecule, making them useful reagents (e.g., biosensors) for diagnostic and testing purposes. DNA aptamer biosensors generally emit a fluorescence signal after target binding to an allosteric site. The emitted fluorescent signal can be dim or bright and is detectable above a certain amount of background fluorescence. In certain instances, a DNA aptamer emits a low signal, such as a dim signal, when a target is bound to the binding site and While useful for certain applications, the current structure of DNA aptamers prevents their use in more complex functionalities. Accordingly, there is a need to identify new DNA aptamers useful as biosensors for a variety of different therapeutic and diagnostic purposes, in vitro and in vivo.

### SUMMARY

The application relates generally to biosensors comprising a reporter and a DNA aptamer with at least one stem. The biosensors disclosed herein may include a DNA aptamer and a reporter. The DNA aptamer is capable of binding a fluorophore reporter such as sulforhodamine-dinitroaniline (SR-DN) and a receptor binding domain (RBD) of a SARS-CoV-2 spike protein. In some embodiments, the DNA aptamer has a sequence that includes at least a portion of a SARS-CoV-2-RBD aptamer backbone and a randomized region. The DNA aptamer can also include one or more primer handles. In such embodiments, the DNA may include a forward primer handle at the 5′ end, a reverse primer handle at the 3′ end, or both a 5′ and a 3′ primer handle.

In certain embodiments, the aptamer includes a SARS-CoV-2-RBD aptamer backbone, such as a SARS-CoV-2-RBD-1C (1C) backbone or a SARS-CoV-2-RBD-4C (4C) backbone (e.g., SEQ ID NO:1, SEQ ID NO:2, SEQ ID NO:3, or SEQ ID NO:4). In some embodiments, the 1C or 4C aptamer backbone is modified to remove at least 5 nucleotides from the 3′ and/or 5′ ends or to remove, insert, or substitute one or more nucleotides.

In certain embodiments, the DNA aptamers disclosed herein comprise any one of SEQ ID NOs:20, 23-42, 89-90. Further, the DNA aptamer may also include a functional group that facilitates attachment to a plate, selected from an NH2 group or a biotin. IN that case, the DNA aptamer may comprise any one of SEQ ID NOs:91-100.

Also disclosed herein according to some embodiments, kits that contain the reagents necessary to perform an assay to diagnose SARS CoV-2 are provided, to include the DNA aptamers discussed above. The it may include one or more of the following: a plate that has immobilized DNA aptamer in the wells of the plate, the detection reagent (SRDN), a buffer, a positive control solution, a negative control solution, a microplate seal, and/or a package insert.

In other embodiments, the present disclosure provides DNA aptamers of the biosensors having a target domain comprising a randomized region of a length of nucleotides within the range of about 15 nucleotides to about 60 nucleotides disposed within the at least one stem, a reporter domain configured to bind to the reporter, and a linker domain operably connected between the target domain and the reporter domain. In certain embodiments, the DNA aptamer comprises a nucleotide sequence of

| (SEQ ID NO: 3) | |
|---|---|
| | NY₁CCGACCTTGTGCTTTGGGAGTGCTGGTCCAAGGGCGTTAANY₂ |

In other embodiments, the DNA aptamer comprises a nucleotide sequence of

(SEQ ID NO: 4)
NY₃ACGCAGCATTTCATCGGGTCCAAAAGGGGCTGCTCGGGATTGCGGAT
ATGGNY₂..

In further embodiments, each of NY₁, NY₂, and NY₃ represents at least a portion of the randomized region.

In some embodiments, the randomized region is about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, or about 60 nucleotides.

In some embodiments, the randomized region is 33 nucleotides. In certain embodiments, NY₁ is 16 nucleotides and NY₂ is 17 nucleotides. In further embodiments, the randomized region of 33 nucleotides is inserted on an outside stem or an inside stem of the aptamer.

In some embodiments, the randomized region is 38 nucleotides. In certain embodiments, NY₃ is 21 nucleotides and NY₂ is 17 nucleotides. In further embodiments, the randomized region of 38 nucleotides is inserted on an outside stem or an inside stem of the aptamer.

In some embodiments, the nucleotide sequence of the randomized region in the target domain that binds the target is identified by SELEX.

In some embodiments, the reporter is a fluorescent molecule.

The present disclosure also provides methods of detecting a target in sample comprising contacting the sample with the biosensors of the present technology. In some embodiments, the target is a pathogen, a small molecule, a solvent, or an ion. In certain embodiments, the pathogen is a bacterial pathogen, a viral pathogen, a prokaryotic pathogen, a fungal pathogen, or a combination thereof. In some aspects, the pathogen is adenovirus, coronavirus, human metapneumovirus, human rhinovirus/enterovirus, influenza, parainfluenza, respiratory syncytial virus, bordatella pertussis, chlamydophia penumoniae, SARS-CoV, SARS-CoV2, MERS-CoV, UPEC, E. coli, Klebsiella pneumoniae, Proteus mirabilis, Pseudomonas aeruginosa, Staphylococcus saprophyticus, Enterococcus faecalis, Enterococcus faecim, Clostridioides difficile, methicillin-resistant Staphylococcus aureus, proteins synthesized by antibiotic resistant bacteria, West Nile virus, Zika virus, Ebola virus, salmonella, equine herpesvirus type 1) and type IV, human immunodeficiency virus (HIV), hepatitis A, hepatitis B, hepatitis C, malaria, Dengue virus, norovirus, rotavirus, astrovirus, Marburg virus, rabies, small pox, measles, or hantavirus. In other aspects, the small molecule is a toxin or a pharmaceutical agent. In certain aspects, the small molecule is a cannabinoid, bisphenol A, fluoride, or benzene. In further aspects, the cannabinoid is cannabidiol, cannabinol, or tetrahydrocannabinol. In some aspects, the solvent is acetone, cyclohexane, acetic acid, ethanol, or benzene. In certain aspects, the ion is potassium, chloride, sodium, lithium, magnesium, mercury, or lead.

### BRIEF DESCRIPTION OF THE DRAWINGS

Many aspects of the present disclosure can be better understood with reference to the following figures. The components in the figures are not necessarily to scale. Instead, emphasis is placed on illustrating clearly the principles of the present disclosure.

FIGS. 1A-1C depict aspects of a 51 nt-3-hairpin structured CoV2-RBD-1C aptamer (“1C aptamer” or “1C”), such as the 1C aptamer backbone sequence with a location of where a randomized region is disposed (FIG. 1A), a computer modeled predictive thermogram for the 1C aptamer 51 nt library with primer handles at 30.0° C. (FIG. 1B) and at 20.0° C. (FIG. 1C) in accordance with embodiments of the present technology.

FIGS. 2A and 2B depict aspects of a 67 nt-hairpin structured CoV2-RBD-4C aptamer (“4C aptamer”), such as the 4C aptamer backbone sequence with a location of where a randomized region is disposed (FIG. 2A) and a predictive thermogram for the 4C aptamer 67 nt library with primer handles at 20.0° C. (FIG. 2B) in accordance with embodiments of the present technology.

FIG. 3 is an example map of a 96-well plate in accordance with some embodiments.

FIG. 4 is an algorithms used in the validated macro-driven Excel spreadsheet according to some embodiments.

FIG. 5 is a screen shot from the validated macro-driven Excel spreadsheet according to some embodiments.

FIG. 6 is a screen shot from the validated macro-driven Excel spreadsheet according to some embodiments.

FIGS. 7A-7B are a screen shots from the validated macro-driven Excel spreadsheet according to some embodiments. FIG. 7A is an exemplary Plate Summary showing results of each well, and FIG. 7B is an exemplary patient summary, which matches each result to the correct patient.

FIG. 8 is a screen shot from the validated macro-driven Excel spreadsheet according to some embodiments.

FIG. 9 is a screen shot from the validated macro-driven Excel spreadsheet according to some embodiments. FIG. 9 shows patient ID sorted by alphabetical order in accordance with the filter and/or sorting functions shown in FIG. 8.

FIG. 10 is a screen shot from the validated macro-driven Excel spreadsheet according to some embodiments. FIG. 9 shows patient ID filtered by positive or negative results in accordance with the filter and/or sorting functions shown in FIG. 8.

FIG. 11 is an example map of a 96-well plate in accordance with some embodiments.

FIG. 12 is a graph showing a standard curve that was generated by making 5, ten-fold serial dilutions of gamma-irradiated SARS-CoV-2 virus in accordance with some embodiments.

FIG. 13 is a scatter dot plot showing the distribution of samples for groups as indicated (positive control, negative control, reference dyt, positive samples, and negative samples), each group superimposed with a floating bar graph showing the minimum and maximum threshold reference levels of relative fluorescent units for that group for Control Results and Sample Interpretation as indicated in Tables 6 and 7.

FIGS. 14A-14C are predicted structures and binding between the SARS-CoV2 spike protein. The aptamer's folding is predicted using RNAcomposer (see Biesiada M, Pachulska-Wieczorek K, Adamiak R W, Purzycka K J. RNAComposer and RNA 3D structure prediction for nanotechnology. Methods. 2016 Jul. 1; 103:120-7. doi: 10.1016/j.ymeth.2016.03.010. Epub 2016 Mar. 24. PMID: 27016145). FIG. 14A illustrates the predicted binding of the N6-D2 aptamer to the spike protein. FIGS. 14B and 14C show models of N6 aptamers (N6 and digested versions of N6: N6-D2, N6-D3, and N6-D4), predicted by RNAcomposer. FIG. 14B shows surface fill models of the N6 aptamers. The predicted spike protein complex binds to the bottom portion of the structures (i.e., target domain) (FIG. 14C), with the SR-DN dye binding elsewhere, likely the center pocket (i.e., reporter domain).

FIG. 15 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN was measured in comparison to controls in the following four wells: H1 (dye only: 3 mg SR-DN powder in 2 mL H2O), H2 (water only), H3 (binding wash buffer, BWB), and H4 (2 uL SRDN at 500 uM in DMSO with 1 mL 1×BWB). No aptamer or SARS-CoV2 spike protein were added to the wells. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 21.6° C.

FIG. 16 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in comparison to controls (water and binding wash buffer) in the following fourwells: H1 (dye only: 3 mg SR-DN powder in 2 mL H2O), H2 (water only), H3 (binding wash buffer, BWB), and H4 (2 uL SRDN at 500 uM in DMSO with 1 mL 1×BWB). 20 μL of 1 μM N1 DNA aptamer was added to each of the wells. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 21.7° C.

FIG. 17 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in comparison to controls (water and binding wash buffer) in the following four wells: H1 (dye only: 3 mg SR-DN powder in 2 mL H2O), H2 (water only), H3 (binding wash buffer, BWB), and H4 (2 uL SRDN at 500 uM in DMSO with 1 mL 1×BWB). 10 μL of dil 6 spike 1 (SARS-CoV2 spike protein) was added to each of the wells. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 21.8° C.

FIG. 18 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following twelve wells: A1 (50 μL SR-DN in H2O), A2 (50 μL SR-DN in DMSO), A3 (dye only), B1 (50 μL SR-DN in H2O+10 μL dil6 Spike 1), B2 (50 μL SR-DN in DMSO+10 μL dil6 Spike 1), B3 (dye+spike protein), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), C3 (SR-DN dye+DNA aptamer), D1 (spike protein+N1), D2 (spike protein+N1), D3 (SR-DN dye+DNA aptamer+spike protein). The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 21.1° C.

FIG. 19 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). The plate reader was set to the following parameters: (i) an excitation wavelength of 575 and an emission of 602 (575, 602), (ii) optics: top, gain: Scale to high wells (D2) (automatic gain values: Gain(575,602): 84), (iii) light source: Xenon Flash, lamp energy: high, (iv) read speed: normal, (v) delay: 100 msec, (vi) measurements/data point: 10, (vii) read height: 6 mm. The experiment was run at a temperature of 22.1° C.

FIG. 20 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). The plate reader was set to the following parameters: (i) an excitation wavelength of 575 and an emission of 602 (575, 602), (ii) optics: top, gain: Scale to low wells (A2) (automatic gain values: Gain(575,602): 50), (iii) light source: Xenon Flash, lamp energy: high, (iv) read speed: normal, (v) delay: 100 msec, (vi) measurements/data point: 10, (vii) read height: 6 mm. The experiment was run at a temperature of 22.1° C.

FIG. 21 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). 50 μL of graphene oxide (GO) was also added to each well. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 22.3° C.

FIG. 22 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). A 50 μL of graphene oxide (GO) was also added to each well, followed by another 50 μL of GO added to each well. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 22.3° C.

FIG. 23 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). A 50 μL of graphene oxide (GO) was also added to each well, followed by another 50 μL of GO, followed by yet another 50 μL of GO. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 23.1° C.

FIG. 24 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the N1 DNA aptamer (see Table 1) was measured in the following eight wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1). The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 23.7° C.

FIG. 25 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of a different DNA aptamer, N4 (see Table 1), was measured in the plate discussed above in FIG. 24 in the following wells: A1 (50 μL SR-DN in H2O), A2 (50 μL SR-DN in DMSO), B1 (50 μL SR-DN in H2O+10 μL dil6 Spike 1), B2 (50 μL SR-DN in DMSO+10 μL dil6 Spike 1), C1 (50 μL SR-DN in H2O+10 μL dil6 Spike 1+20 μL 1 μM N1), C2 (50 μL SR-DN in DMSO+10 μL dil6 Spike 1+20 μL 1 μM N1), D1 (spike protein+N1), D2 (spike protein+N1), E1 (50 μL SR-DN in H2O), E2 (50 μL SR-DN in DMSO), F1 (50 μL SR-DN in H2O+10 μL dil6 Spike 1), F2 (50 μL SR-DN in DMSO+10 μL dil6 Spike 1), G1 (50 μL SR-DN in H2O+10 μL dil6 Spike 1+20 μL 1 μM N4), G2 (50 μL SR-DN in DMSO+10 μL dil6 Spike 1+20 μL 1 μM N4), H1 (spike protein+N1), H2 (spike protein+N1). The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 23.8° C.

FIG. 26 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. 100 μL of GO was also added to each cell. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 23.9° C.

FIG. 27 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.1° C.

FIG. 28 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.2° C.

FIG. 29 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: Scale to high wells (D2) (Gain(575,602): 84, (iii) light source: Xenon Flash, lamp energy: high, (iv) read speed: normal, (v) delay: 100 msec, (vi) measurements/data point: 10, (vii) read height: 6 mm. The experiment was run at a temperature of 24.3° C.

FIG. 30 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: Scale to high wells (D2) (Gain(575,602): 84, (iii) light source: Xenon Flash, lamp energy: high, (iv) read speed: normal, (v) delay: 100 msec, (vi) measurements/data point: 10, (vii) read height: 6 mm. The experiment was run at a temperature of 24.4° C.

FIG. 31 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: Scale to high wells (D2) (Gain(575,602): 84, (iii) light source: Xenon Flash, lamp energy: high, (iv) read speed: normal, (v) delay: 100 msec, (vi) measurements/data point: 10, (vii) read height: 6 mm. The experiment was run at a temperature of 24.5° C.

FIG. 32 is an excel chart with 96 cells, which correspond to the 96 wells of a microplate used for an experiment to detect SARS-CoV2 spike proteins by a DNA aptamer according to some embodiments. Fluorescence emitted by the fluorophore SR-DN in the presence of the DNA aptamers, N1 and N4 (see Table 1), was measured in the plate discussed above in FIG. 25. The plate reader was set to the following parameters: (i) an excitation wavelength of 575 and an emission of 602 (575, 602), (ii) optics: top, gain: Scale to high wells (D2) (Gain(575,602): 84, (iii) light source: Xenon Flash, lamp energy: high, (iv) read speed: normal, (v) delay: 100 msec, (vi) measurements/data point: 10, (vii) read height: 6 mm. The experiment was run at a temperature of 24.5° C.

FIG. 33 is an excel chart with 96 cells, which correspond to the 96 wells of a microplate used for an experiment to detect MERS and SARS-CoV2 spike proteins (S1, S2) by a DNA aptamer according to some embodiments (TO). Fluorescence emitted by the fluorophore SR-DN in the presence of DNA aptamers (N1 and/or N4), was measured and recorded in the excel chart. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.6° C.

FIG. 34 is an excel chart with 96 cells, which correspond to the 96 wells of a microplate used for an experiment to detect MERS and SARS-CoV2 spike proteins (S1, S2) by a DNA aptamer according to some embodiments (T1). Fluorescence emitted by the fluorophore SR-DN in the presence of DNA aptamers (N1 and/or N4), was measured and recorded in the excel chart. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.6° C.

FIG. 35 is an excel chart with 96 cells, which correspond to the 96 wells of a microplate used for an experiment to detect MERS and SARS-CoV2 spike proteins (S1, S2) by a DNA aptamer according to some embodiments (T5). Fluorescence emitted by the fluorophore SR-DN in the presence of DNA aptamers (N1 and/or N4), was measured and recorded in the excel chart. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.6° C.

FIG. 36 is an excel chart with 96 cells, which correspond to the 96 wells of a microplate used for an experiment to detect MERS and SARS-CoV2 spike proteins (S1, S2) by a DNA aptamer according to some embodiments (T30). Fluorescence emitted by the fluorophore SR-DN in the presence of DNA aptamers (N1 and/or N4), was measured and recorded in the excel chart. The plate reader was set to the following parameters: (i) an excitation wavelength of 575 and an emission of 602 (575, 602), (ii) optics: top, gain: 100, (iii) light source: Xenon Flash, lamp energy: high, (iv) read speed: normal, (v) delay: 100 msec, (vi) measurements/data point: 10, (vii) read height: 7 mm. The experiment was run at a temperature of 24.6° C.

FIGS. 37-39 are a summary of FIGS. 33-36, and analyses including a comparison of signals between S1, S2, and MERS spike proteins. Over time signal increases for S2 when compared to S1. The S1:S2 ratio decreases over time indicating preferential binding for S1 over S2. MERS is significantly brighter than both S1 and S2, which may be due to S1/S2 decreasing more than MERS or becoming less bright.

### DETAILED DESCRIPTION

The present technology is directed to biosensors comprising an aptamer (e.g., an aptamer having a target domain, a reporter domain, and a linker domain) and a reporter for determining the presence of a target in a sample, and associated systems and methods of use. Some embodiments of the present technology, for example, are directed to biosensors having aptamers that, upon binding of the target to the target domain, undergo a conformational change resulting in a change in signal emission, reduced or enhanced signal emission, signal quenching, or enhanced signal quenching by the reporter (e.g., a second signal or a second state) compared to a signal emitted when the aptamer is not bound to the target (e.g., a first signal or a first state). The conformational change can be an allosteric change.

In some embodiments, the DNA aptamers disclosed herein are based on an aptamer backbone (“1C aptamer” or “1C”) against the receptor binding domain (RBD) of SARS-CoV-2 spike glycoprotein

SEQ ID NO: 1
(CAGCACCGACCTTGTGCTTTGGGAGTGCTGGTCCAAGGGCGTTAATGGA
CA,)

or a SARS-CoV-2-RBD-4C aptamer backbone (“4C aptamer” or “4C”) against the RBD of SARS-CoV-2 spike glycoprotein

SEQ ID NO: 2
(ATCCAGAGTGACGCAGCATTTCATCGGGTCCAAAAGGGGCTGCTCGGGA
TTGCGGATATGGACACGT,),

and include a length of randomized nucleic acids within at least one stem of the 1C or the 4C aptamer backbone. Both the 1C and the 4C aptamer backbones were previously identified by Song et. al. (Song, Yanling: Song, Jia; Wei, Xinyu: Huang, Mengjiao; Sun, Miao; Zhu, Lin; et al. (2020): Discovery of Aptamers Targeting Receptor-Binding Domain of the SARS-CoV-2 Spike Glycoprotein. ChemRxiv. Preprint. https://doi.org/10.26434/chemrxiv.12053535.v1). In some embodiments, the DNA aptamers disclosed herein comprise a nucleotide sequence of

(SEQ ID NO: 3)
NY₁CCGACCTTGTGCTTTGGGAGTGCTGGTCCAAGGGCGTTAANY₂
or
(SEQ ID NO: 4)
NY₃ACGCAGCATTTCATCGGGTCCAAAAGGGGCTGCTCGGGATTGCGGAT
ATGGNY₂.

In certain embodiments, each of NY₁, NY₂, and NY₃ represents at least a portion of the randomized region. The length of the randomized region can be selected based on the target of interest and specific aptamer sequences including the randomized region identified using SELEX. In some embodiments, the randomized region is about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, or about 60 nucleotides. For example, in some embodiments, while not intending to be limiting, the randomized region is 33 or 38 nucleotides and NY₁ is 16 nucleotides and NY₂ is 17 nucleotides or NY₃ is 21 nucleotides and NY₂ is 17 nucleotides.

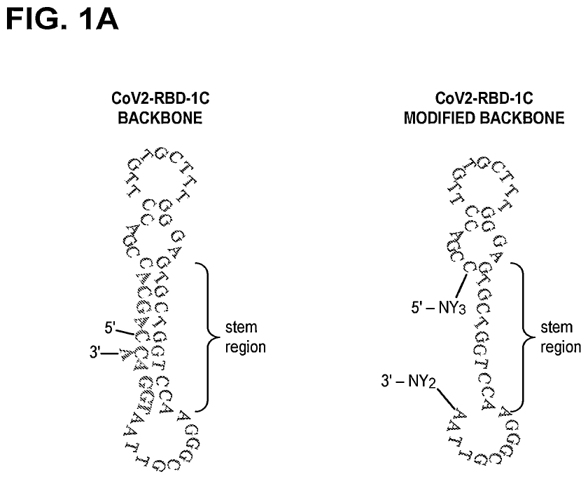

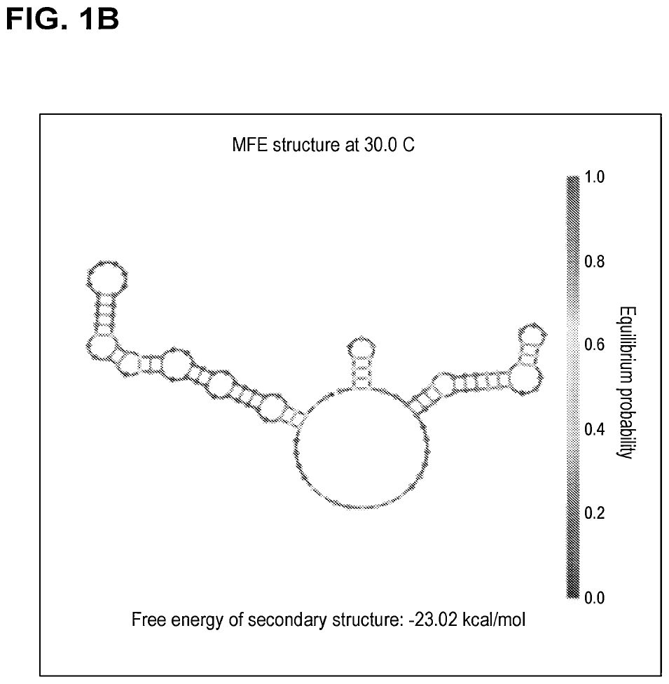

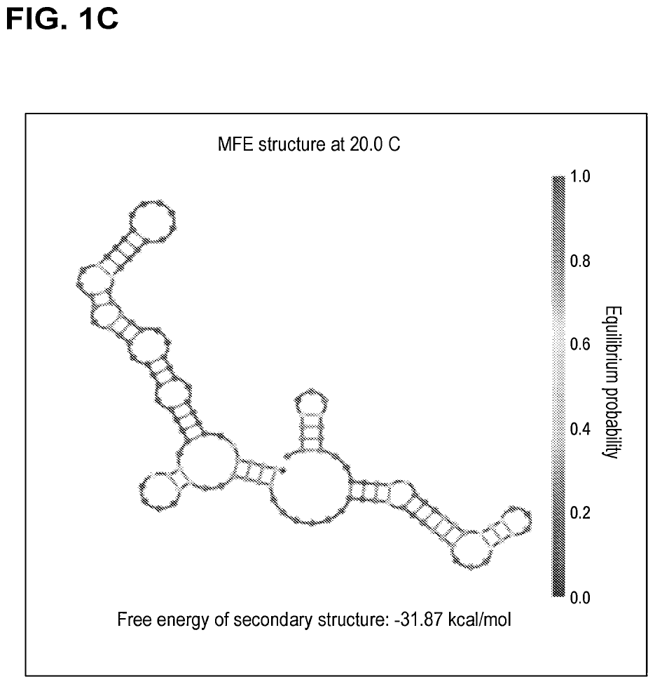

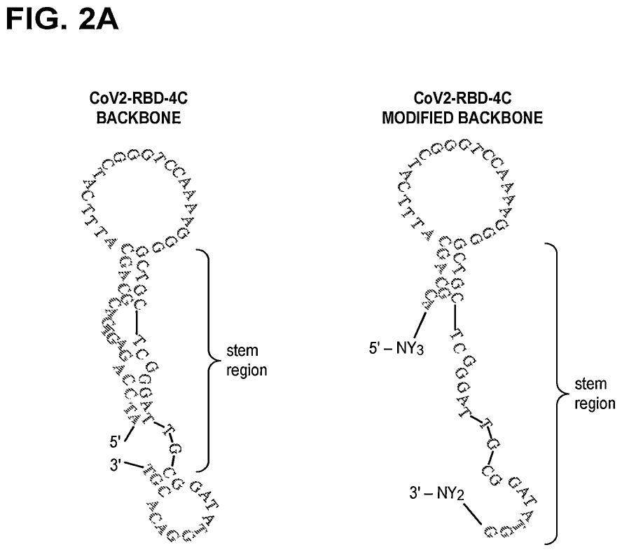

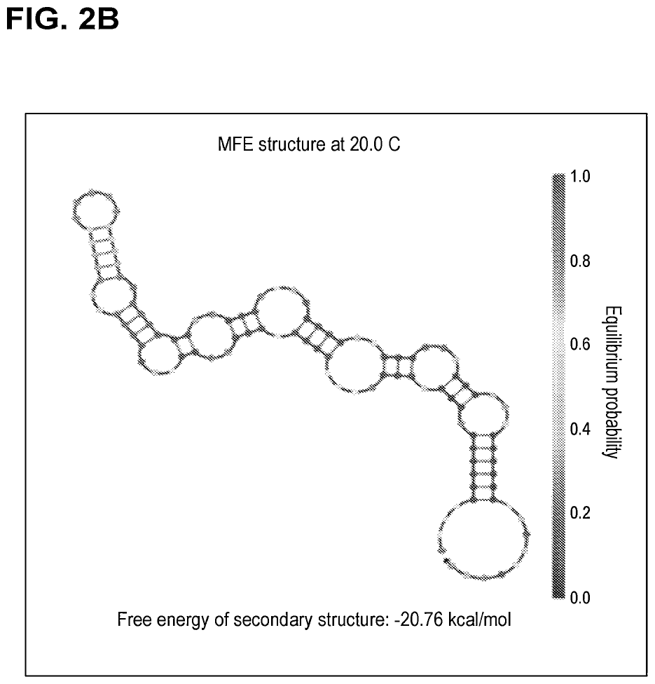

Useful reporters include fluorescent reporters, such as sulforhodamine-dinitroaniline (SR-DN), as discussed below. Binding of the aptamer to the target can be detected by a change between a first signal emitted in a target unbound state and a second signal emitted in a target bound state. In some embodiments, the first signal has a greater intensity than the second signal. In other embodiments, the second signal has a greater intensity than the first signal. In either of these embodiments, the signal is determined after quenching has been detected. These aptamers and reporters, and other aptamers and reporters derived from and/or otherwise based upon the aptamers and reporters described herein, are included in embodiments of the present technology. Specific details of several embodiments of the technology are described below with reference to FIGS. 1A-2B.

While the present technology is capable of being embodied in various forms, the description below of several embodiments is made with the understanding that the present disclosure is to be considered as an exemplification of the technology and is not intended to limit the technology to the specific embodiments illustrated. Headings are provided for convenience only and are not to be construed to limit the technology in any manner. Embodiments illustrated under any heading may be combined with embodiments illustrated under any other heading.

In the present description, any concentration range, percentage range, ratio range, or integer range is to be understood to include the value of any integer within the recited range and, when appropriate, fractions thereof (such as one tenth and one-hundredth of an integer), unless otherwise indicated. Also, any number range recited herein is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present technology has been described in terms of particular embodiments found or proposed by the present inventor to comprise preferred modes for the practice of the technology. It will be appreciated by those of skill in the art that, in light of the present disclosure, numerous modifications and changes can be made in the particular embodiments exemplified without departing from the intended scope of the technology. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Unless otherwise defined, all technical and scientific terms used herein have the same meaning as commonly understood by one of ordinary skill in the art to which this disclosure pertains. Although methods and materials similar or equivalent to those described herein can be used in the practice of the present disclosure, suitable methods and materials are described below. Unless otherwise indicated, nucleic acids are written left to right in 5′ to 3′ orientation; amino acid sequences are written left to right in amino to carboxy orientation, respectively. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

### Definitions

The term “biosensor”, as used herein, refers to a macromolecule comprising an aptamer and a reporter, and optionally, one or more linkers.

As used herein, the term “aptamer” refers to any polynucleotide, generally a DNA, that has a useful biological activity in terms of biochemical activity, molecular recognition or binding attributes to a target. Usually, an aptamer has a molecular activity such as binding to a target at a specific binding site on the target. It is generally accepted that an aptamer, which is specific in its binding to the target, may be synthesized and/or identified by SELEX. Aptamers of the present technology often include two binding sites, a target binding site and a reporter binding site.

The term “systematic evolution of ligands by exponential enrichment” or “SELEX” generally means any method of selecting for an aptamer which binds to a target. SELEX involves screening a pool of random targets for a particular aptamer that binds to a target or has a particular activity that is selectable. Generally, the particular aptamer represents a very small fraction of the target pool, therefore, a round of aptamer amplification, usually via polymerase chain reaction, is employed to increase the representation of potentially useful aptamers. Successive rounds of selection and amplification are employed to exponentially increase the abundance of the particular and useful aptamer. SELEX is described in several publications including, but not limited to, Famulok, M.; Szostak, J. W., In Vitro Selection of Specific Ligand Binding Nucleic Acids, Angew. Chem. 1992, 104, 1001. (Angew. Chem. Int. Ed. Engl. 1992, 31, 979-988); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20, 97-107; and Burgstaller, P.; Famulok, M. Synthetic ribozymes and the first deoxyribozyme; Angew. Chem. 1995, 107, 1303-1306 (Angew. Chem. Int. Ed. Engl. 1995, 34, 1189-1192).

As used herein, the terms “antigen,” “target” and “analyte” are used interchangeably and refer generally to a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or any other agent which is capable of binding to an aptamer of the present technology. A target is characterized by its ability to be “bound” by the aptamer. Target can also mean the substance used to elicit the production of targeting moieties, such as the production of aptamers through immunizing with the target.

The term “antigen binding site,” “target binding site,” “analyte brining site,” or “epitope” refers to the portion of the target to which the aptamer binds.

The terms “bind,” “binds,” and “specifically binds” refers to the ability of an aptamer to bind to a target with greater affinity than it binds to a non-target. In certain embodiments, specific binding refers to binding for aptamer with an affinity that is at least 10, 50, 100, 250, 500, or 1000 times greater than the affinity for a non-target.

The term “binding affinity” refers to the strength of interaction between an aptamer and its target as a function of its association and dissociation constants. Higher affinities typically mean that the aptamer has a fast on rate (association) and a slow off rate (dissociation). Binding affinities can change under various physiological conditions due to changes that occur to the target or aptamer under those conditions. Binding affinities of the aptamer can also change when a reporter is attached. Binding affinities can also change when slight changes occur to the target, such as changes in the amino acid or nucleotide sequence or glycosylation of the target. Generally, the aptamers of the present disclosure have high binding affinities for their respective targets.

The term “linker” or “linker molecule” refers to any polymer attached to an aptamer or aptamer construct. The attachment may be covalent or non-covalent. It is envisioned that the linker can be a polymer of amino acids or nucleotides. A preferred linker molecule is flexible and does not interfere with the binding of a nucleic acid binding factor to the set of nucleic acid components.

The term “reporter,” as used herein, refers to any substance attachable (e.g., by binding) to an aptamer in which the substance is detectable by a detection method. Non-limiting examples of reporters applicable to this technology include but are not limited to luminescent molecules, chemiluminescent molecules, fluorochromes, fluorescent quenching agents, colored molecules, radioisotopes, mass reporters, biotin, avidin, streptavidin, protein A, protein G, antibodies or fragments thereof, polyhistidine, nickel and its ions, Flag tags, myc tags, heavy metals, enzymes, alkaline phosphatase, peroxidase, luciferase, and colorimetric substrates.

As used herein, the terms “detection method” and “detectable signal” are interchangeable and refer to any method or output of the method known in the art to detect a molecular interaction event, such as binding of an aptamer to a target. Non-limiting examples of detection methods include detecting changes in fluorescence (e.g., FRET, FCCS, decreasing fluorescence, such as fluorescence quenching, or increasing fluorescence, fluorescence polarization), changes in mass, changes in enzymatic activity, and changes chemiluminescence.

The term “effective amount” refers to an amount of an aptamer, either alone or as a part of a composition, such as a biosensor or other composition, that is capable of having any detectable output, such as a detectable signal when combined with a sample, or a therapeutic effect on any symptom, aspect, parameter or characteristics of a disease state or condition when administered to a subject. Such effect need not be absolute to be detectable and/or beneficial.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, zoo animals, animals used in sporting events (e.g., racehorses), or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L, and norleucine (NIe).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gin or Q); Group 4: Arginine (Arg or R), Lysine (Lys or K), Histidine (His or H); Group 5: Isoleucine (lie or 1), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine (A) and guanine (G), and pyrimidine bases include uracil (U), thymine (T), and cytosine (C). Unless otherwise indicated, the aptamers disclosed herein are generally composed of polydeoxyribonucleic acid (DNA) molecules, which includes cDNA, genomic DNA, and synthetic DNA, any of which may be single or double-stranded. A nucleic acid molecule encoding an amino acid sequence includes all nucleotide sequences that encode the same amino acid sequence. In addition to the single letter codes for the nucleotides A, G, U, T, C, nucleotide sequences may use IUPAC single letter codes to designate more than one nucleotide alternative/ambiguous sequence, e.g., R (A or G); Y (C or T/U); M (A or C); K (G or T); S (C or G); W (A or T); H (A or C or T); B (C or G or T); V (A or C or G); D (A or G or T); or N (A or C or G or T).

The terms “nucleotide” and “nucleic acid” are used interchangeably and refer to any nucleoside linked to a phosphate group. Nucleic acids in accordance with the embodiments described herein may include nucleotides entirely of the types found in naturally occurring nucleic acids, or may instead include one or more nucleotide analogs or have a structure that otherwise differs from that of a naturally occurring nucleic acid. U.S. Pat. Nos. 6,403,779, 6,399,754, 6,225,460, 6,127,533, 6,031,086, 6,005,087, 5,977,089, disclose a wide variety of specific nucleotide analogs and modifications that may be used, and are hereby incorporated by reference as if fully set forth herein. Also see Crooke, S. Antisense Drug Technology: Principles, Strategies, and Applications (1st ed), Marcel Dekker; ISBN: 0824705661; 1st edition (2001), which is also hereby incorporated by reference as if fully set forth herein. For example, the nucleoside may be natural, including but not limited to, any of cytidine, uridine, adenosine, guanosine, thymidine, inosine (hypoxanthine), or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Nucleic acids having a variety of different nucleotide analogs, modified backbones, or non-naturally occurring internucleoside linkages can be utilized in accordance with the embodiments described herein.

Nucleic acids may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurne, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluoroibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available.

Alternatively, a nucleotide may be abasic, such as but not limited to 3-hydroxy-2-hydroxymethyl-tetrahydrofuran, which act as a linker group lacking a base or be a nucleotide analog. 2-modifications include halo, alkoxy and allyloxy groups, and the 2′-OH group can be replaced by a group selected from H, OR, R, halo, SH, SR, NH₂, NHR, NR₂ or CN, wherein R is C1-C6 alkyl, alkenyl, or alkynyl, and halo is F, Cl, Br, or I.

Non-limiting examples of synthetic bases and analogs include, but are not limited to methyl-substituted phenyl analogs, such as but not limited to mono-, di-, tri-, or tatramethylated benzene analogs; hydrophobic base analogs, such as but not limited to 7-propynyl isocarbostyril nucleoside, isocarbostyril nucleoside, 3-methylnapthalene, azaindole, bromo phenyl derivates at positions 2, 3, and 4, cyano derivatives at positions 2, 3, and 4, and fluoro derivates at position 2 and 3; purine/pyrimidine mimics, such as but not limited to azole hetercyclic carboxamides, such as but not limited to (1H)-1,2,3-triazole-4-carboxamide, 1,2,4-triazole-3-carboxamide, 1,2,3-trazole-4-carboxamide, or 1,2-pyrazole-3-carboxamide, or heteroatom-containing purine mimics, such as furo or theino pyridiones, such as but not limited to furo[2,3-c]pyridin-7(6H)-one, thieno[2,3-c]pyridin-7(6H)-one, furo[2,3-c]pyridin-7-thiol, furo[3,2-c]pyridin-4(5H)-one, thieno[3,2-c]pyridin-4(5H)-one, or furo[3,2-c]pyridin-4-thiol, or other mimics, such as but not limited to 5-phenyl-indolyl, 5-nitro-indolyl, 5-fluoro, 5-amino, 4-methylbenzimidazole, 6H,8H-3,4-dihydropropyrimido[4,5-c][1,2]oxazin-7-one, or N6-methoxy-2,6-diaminopurine; isocytosine, isoquanosine; thymidine analogs, such as but not limited to 5-methylisocytosine, difluorotoluene, 3-toluene-1-β-D-deoxyriboside, 2,4-difluoro-5-toluene-1-β-D-deoxyriboside, 2,4-dichloro-5-toluene-1-β-D-deoxyrboside, 2,4-dibromo-5-toluene-1-β-D-deoxyriboside, 2,4-diiodo-5-toluene-1-β-D-deoxyriboside, 2-thiothymidine, 4-Se-thymidine; or fluorescent base analogs, such as but not limited to 2-aminopurine, 1,3-diaza-2-oxophenothiazine, 1,3-diaza-2-oxophenoxazine, pyrrolo-dC and derivatives, 3-MI, 6-MI, 6-MAP, or furan-modified bases, phosporothioate nucleotides, 2′-O-methyl ribonucleotides, 2′-O-methoxy-ethyl ribonucleotides, peptide nucleotides, N3′-P5′ phosphoroamidate, 2′-fluoro-arabino nucleotides, locked nucleotides (LNA), unlocked nucleotides (UNA), morpholino phosphoroamidate, cyclohexene nucleotides, tricyclo-deoxynucleotides, or triazole-linked nucleotides. Examples of modified linkages include phosphorothioate and 5′-N-phosphoramidite linkages.

Modified nucleic acids need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially affected. Non-limiting examples of modifications are located at any position of an aptamer component such that the ability of the aptamer to specifically bind to the target is not substantially affected. The modified region may be at the 5′-end and/or the 3′-end of one or both strands. For example, modified nucleic acid aptamers in which approximately 1-5 residues at the 5′ and/or 3′ end of either of both strands are nucleotide analogs and/or have a backbone modification have been employed. The modification may be a 5′ or 3′ terminal modification.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “nucleotide duplex” is when two strands of complement nucleotide oligomers complementary bind to each other. The two strands may be part of the same nucleotide molecule or separate nucleotide molecules.

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

A “receptor” may be peptides, proteins, glycoproteins, lipoproteins, epitopes, antibodies, lipids, carbohydrates, multi-molecular structures, a specific conformation of one or more molecules and a morphoanatomic entity that has a binding affinity for a specific group of chemicals or molecules, such as other proteins or viruses. Upon recognition and binding of the chemical or molecule, the receptor can cause some form of signaling or other process within a cell to respond to the chemical or molecule. Optionally, the chemical or molecule can cause a receptor to stop functioning property and shut off processes.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “expression vector” refers to a nucleic acid construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors). The terms should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; U.S. Pat. No. 5,591,439). Examples of non-viral vectors include, but are not limited to, liposomes, polyamine derivatives of DNA, and the like. Once transformed into a suitable host, the vector may replicate and function independently of the host genome, or may, in some instances, integrate into the genome itself. Here, “plasmid,” “expression plasmid,” “virus,” and “vector” are often used interchangeably. The terms refer broadly to any plasmid or virus encoding an exogenous nucleic acid.

As used herein “promoter” includes reference to a region of DNA upstream from the start of transcription and involved in recognition and binding of RNA polymerase and other proteins to initiate transcription.

The term “introduced” in the context of inserting a nucleic acid molecule into a cell means “transfection,” “transformation,” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, enzymes, or other nucleic acid molecule additions, deletions, substitutions, or other functional disruption of a cell's genetic material.

The term “construct” refers to any polynucleotide that contains a recombinant nucleic acid molecule. A construct may be present in a vector (e.g., a bacterial vector, a viral vector) or may be integrated into a genome.

The term “host cell”, as used herein, includes any cell type which is susceptible to transformation with a nucleic acid construct. By “host cell” is meant a cell which contains a vector and supports the replication and/or expression of the vector. Host cells may be prokaryotic cells such as E. coli, or eukaryotic cells such as yeast, insect, amphibian, or mammalian cells.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

A “subject in need thereof” as used herein refers to a mammalian subject, preferably a human, who has been diagnosed with a condition, is suspected of having a condition, and/or exhibits one or more symptoms or risk factors associated with a condition.

The terms “treating” and “treatment” in relation to a given condition, disease, or disorder are used interchangeably and include, but are not limited to, inhibiting the disease or disorder, for example, arresting the development or rate of development of the condition, disease, or disorder; relieving the condition, disease, or disorder, for example, causing regression of the condition, disease, or disorder; or relieving a condition caused by or resulting from the disease or disorder, for example, arresting, relieving, preventing, or causing regression of at least one of the symptoms of the disease or disorder.

The terms “preventing” and “prevention” in relation to a given condition, disease, or disorder are used interchangeably and include, but are not limited to, preventing or delaying the onset of its development if none had occurred; preventing or delaying the condition, disease, or disorder from occurring in a subject that may be predisposed to the condition, disease, or disorder but has not yet been diagnosed as having the condition, disease; or disorder, and/or preventing or delaying further development of the condition, disease, or disorder if already present.

As used herein, “route” in relation to administration of one or more therapies, such as a therapeutic agent (e.g., drug), refers to a path by which the therapeutic agent is delivered to a subject, for example, a subject's body. A route of therapeutic administration include enteral and parenteral routes of administration. Enteral administration includes oral, rectal, intestinal, and/or enema. Parenteral includes topical, transdermal, epidural, intracerebral, intracerebroventricular, epicutaneous, sublingual, sublabial, buccal, inhalational (e.g., nasal), intravenous, intraarticular, intracardiac, intradermal, intramuscular, intraocular, intraosseous infusion, intraperitoneal, intrathecal, intravitreal, subcutaneous, perivascular, implantation, vaginal, otic, and/or transmucosal.

While the present technology is capable of being embodied in various forms, the description below of several embodiments is made with the understanding that the present disclosure is to be considered as an exemplification of the technology and is not intended to limit the technology to the specific embodiments illustrated.

### Biosensors

Biosensors of the present technology generally include a reporter and an aptamer, the aptamer having a target region to bind a target (e.g., a SARS-CoV spike protein), a reporter region that binds a reporter (e.g., SR-DN), and a linker region between the target and reporter regions. Upon hybridization of a target with a target domain in the aptamer, the aptamer undergoes a conformational change (e.g., allosteric change) through a communication domain resulting in a change in signal emission, reduced or enhanced signal emission, by the reporter compared to a state when the aptamer is not bound to the target, i.e., as compared to a control. The emitted signal correlates to the presence of the target, such as identifying the target in the sample. In some embodiments, the emitted signal for the target (e.g., SARS-CoV2 spike protein) is less than a signal emitted by a control, such as a target in the same class yet having a different structure (e.g., MERS spike protein). In other embodiments, the emitted signal for the target is greater than a signal emitted by the control. Non-limiting examples of controls include phosphate buffered saline or a background signal generated by the fluorescent dye emitted at an emission wavelength followed by an excitation wavelength. Regardless of the embodiments, biosensors of the present technology are logic gated and binding to the target is compared to the control to determine presence or absence of the target in a sample which can be indicated by increased or decreased fluorescent signal upon target binding compared to the control. In some embodiments, binding of the biosensors of the present technology to the target is indicated by decreased fluorescent signal upon target binding compared to the control. For example, in samples form subjects having SARS-CoV2, decreased fluorescent signal upon target binding to the biosensors of the present technology is detected compared to a control (e.g, subjects from patients not having a SARS-CoV2 infection) is observed.

In some embodiments, biosensors of the present technology are modular with one or more regions configured to be replaced with a different region of the same type. For example, a first aptamer region may be replaced with a second aptamer region that may or may not bind the same target as the first aptamer region. As another example, a first reporter region may be replaced with a second reporter region. This modular configuration of the biosensors of the present technology results in a plug-and-play design approach making the biosensors rapidly and efficiently adaptable for use in multiple different applications with minimal design change. In some embodiments, the biosensors are useful as a logic gate. Components of the biosensors, additional features of the biosensors, methods of preparing the biosensors, and methods of using the biosensors are described in greater detail below.

### Aptamers

Aptamers useful with biosensors of the present technology include at least three domains: a sensor domain configured to bind a target at a target binding site within the sensor domain, a reporter domain configured to bind a reporter at a reporter binding site within the reporter domain, and a linker. The aptamer is allosteric, resulting in a conformational change once the target binding site is bound to the target.

Aptamers useful with biosensors of the present technology include one or more strands of oligonucleotides including, but not limited to, DNA. In other embodiments, one or more non-DNA nucleic acids, such as RNA, PNA, LNA, and UNA may be included in one or more strands of oligonucleotides to change a physical property of the biosensor, such as a rigidity of the biosensor. For example, UNA may be used to make a more relaxed backbone while LNA may make a more rigid backbone compared to a biosensor comprised solely of DNA.

In certain embodiments, the aptamers described herein include a DNA aptamer backbone that has a predetermined or known sequence. In some embodiments, the backbone is an aptamer having a publicly available sequence, such as but not limited to 1C (FIG. 1A, left panel) or 4C (FIG. 2A, left panel). The nucleotide sequence of 1C is

(SEQ ID NO: 1)
CAGCACCGACCTTGTGCTTTGGGAGTGCTGGTCCAAGGGCGTTAATGGAC
A.

The nucleotide sequence of 4C is

(SEQ ID NO: 2)
ATCCAGAGTGACGCAGCATTTCATCGGGTCCAAAAGGGGCTGCTCGGGAT
TGCGGATATGGACACGT.

In other embodiments, the backbone is an aptamer having a sequence that is not publicly known. In certain embodiments, the backbone is modified. Non-limiting examples of modifications that may be made to a backbone include modifications to one or more nucleotides or nucleosides; modifications to one or more nucleotide linkages; modifications to the sequence of the backbone (e.g., one or more nucleic acid molecule additions, deletions, substitutions); or a combination thereof.

In some embodiments, the aptamers described herein include a DNA aptamer backbone that is modified to remove 11 nucleotides from the from the original 1C aptamer (5 nucleotides on the 5′ end and 6 nucleotides on the 3′ end) to improve the stability of the final structure of the aptamers described herein, and resulting in a nucleotide sequence of CCGACCTTGTGCTTTGGGAGTGCTGGTCCAAGGGCGTTAA (SEQ ID NO:5)). In other embodiments, the aptamers described herein include a DNA aptamer backbone that is modified to remove 16 nucleotides from the from the original 4C aptamer (10 nucleotides on the 5′ end and 6 nucleotides on the 3′ end) to improve the stability of the final structure of the aptamers described herein, and resulting in a nucleotide sequence of ACGCAGCATTTCATCGGGTCCAAAAGGGGCTGCTCGGGATTGCGGATATGG (SEQ ID NO:6)). The aptamer may have fewer or additional truncations of the 1C aptamer (e.g., 8, 9, 10, 11, 12, 13, 14, or more that 15 nucleotides may be removed) or 4C aptamer (e.g., 10 11, 12, 13, 14, 15, 16, 17, 18, 19, 20, or more that 20 nucleotides may be removed). In addition to improving the stability of the structure, truncation of the aptamer backbone makes the aptamer easier to synthesize, and is also less expensive to produce.

Target domains of the aptamers include randomized regions at one or more stems of the backbone. For example, the aptamers may include one or more randomized regions that is (a) inserted into a stem region of the backbone, (b) replaces all or a portion of a stem region of the backbone, and/or (c) is added to the 3′-terminal, to the 5′-terminal, or to both the 3′- and the 5′-terminals of the backbone. Without intending to be limiting, the randomized regions of the target domains are thought to confer specificity for the target bound by the target domain. As described herein, SELEX can be used to identify aptamers useful for binding to a particular target domain.

The length of the randomized region can be selected based on a size of the target that the aptamer is sought to bind and having a desired signal intensity after binding to the target. For example, smaller targets, such as short oligonucleic acids, short peptides, and ions, may be bound by aptamers having shorter randomized regions (e.g., under 20 nucleic acids). As another example, larger targets, such as larger proteins (e.g., spike proteins) may be bound by aptamers having longer randomized regions (e.g., more than 20 nucleic acids). In some embodiments, the randomized region or regions include at least 10, at least 20, at least 30, at least 40, at least 50, or at least 60 nucleotides, for example, the randomized region is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 51 nucleotides, about 52 nucleotides, about 53 nucleotides, about 54 nucleotides, about 55 nucleotides, about 56 nucleotides, about 57 nucleotides, about 58 nucleotides, about 59 nucleotides, about 60 nucleotides, or more than 60 nucleotides. In certain embodiments, of the randomized region or regions nucleotide lengths are about 60 (e.g. NNNNNNNNNNNNNNNNNNNNNNNNNNNNNNNNNNNNNNNNNNNNNNNNNNNNNNN NNNNN, SEQ ID NO:7), about 33 (e.g., NNNNNNNNNNNNNNNNNNNNNNNNNNNNNNNNN, SEQ ID NO:8), about 34 (e.g., NNNNNNNNNNNNNNNNNNNNNNNNNNNNNNNNNN, SEQ ID NO:9), about 38 (e.g., NNNNNNNNNNNNNNNNNNNNNNNNNNNNNNNNNNNNNN, SEQ ID NO:10), about 21 (e.g., NNNNNNNNNNNNNNNNNNNNN, SEQ ID NO:11), about 19 (e.g., NNNNNNNNNNNNNNNNNNN, SEQ ID NO:12), about 17 (e.g., NNNNNNNNNNNNNNNNN, SEQ ID NO:13), about 16 (e.g., NNNNNNNNNNNNNNNN, SEQ ID NO:14), about 15 (e.g., NNNNNNNNNNNNNNN, SEQ ID NO:15), about 14 (e.g., NNNNNNNNNNNNNN, SEQ ID NO:16), or about 13 (e.g., NNNNNNNNNNNNN, SEQ ID NO:17) nucleotides. In certain aspects, a randomized region may be split into two separate regions as shown in the nucleotide sequences herein. Non-limiting examples include (i) a randomized region including about 38 nucleotides (38mer) may be split into a first nucleotide that is 21 nucleotides in length (21mer) and a second nucleotide that is 17 nucleotides in length, (ii) a randomized region including about 33 nucleotides (33mer) may be split into a first nucleotide that is 16 nucleotides in length (16mer) and a second nucleotide that is 17 nucleotides in length (17mer); (iii) a randomized region including about 34 nucleotides (34mer) may be split into a first nucleotide that is 21 nucleotides in length (21mer) and a second nucleotide that is 13 nucleotides in length (13mer).

The randomized regions can be located (e.g., inserted, replaced or added to) on any stem of the aptamer backbone and at any location within the stem. In certain embodiments, the randomized region or regions are added to the 3′-terminal, to the 5′-terminal, or to both the 3′- and the 5′-terminals of the backbone. For example, as shown in FIG. 1A, randomized regions can be inserted at position “NY₁” and “NY₂” of the following sequence:

| (SEQ ID NO: 3) | |
|---|---|
| | NY₁CCGACCTTGTGCTTTGGGAGTGCTGGTCCAAGGGCGTTAANY₂ |

according to some embodiments (see, e.g., FIG. 1A, right panel). As another example and as illustrated in FIG. 2A, randomized regions can be inserted at position “NY₃” and “NY₂” of the following sequence:

(SEQ ID NO: 4)
NY₃ACGCAGCATTTCATCGGGTCCAAAAGGGGCTGCTCGGGATTGCGGAT
ATGGNY₂

according to some embodiments. A non-limiting exemplary sequence for an aptamer having a randomized region of 33 nucleotides is:

(SEQ ID NO: 18)
NNNNNNNNNNNNNNNNCCGACCTTGTGCTTTGGGAGTGCTGGTCCAAGGG
CGTTAANNNNNNNNNNNNNNNNN,

where NY₁ is a randomized region of 16 nucleotides and NY₂ is a randomized region of 17 nucleotides. A non-limiting exemplary for an aptamer having a randomized region of 38 nucleotides is:

(SEQ ID NO: 19)
NNNNNNNNNNNNNNNNNNNNNACGCAGCATTTCATCGGGTCCAAAAGGGG
CTGCTCGGGATTGCGGATATGGNNNNNNNNNNNNNNNNN,

where NY₃ is a randomized region of 21 nucleotides and NY₂ is a randomized region of 17 nucleotides.

In other embodiments, the backbone of the aptamer is wholly composed of a randomized region of nucleotides (e.g., about 65, about 64, about 60, about 59, about 58, about 57, about 56, about 55, about 54, about 53, about 52, about 51, about 50, about 49, about 48, about 47, about 46, about 45, about 44, about 43, about 42, about 41, about 40, about 39, about 38, about 37, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10).

In some embodiments, the aptamers described herein also include one or more primer handle sequences (i.e., the recognition site for primers to bind to the aptamer) on the 5′ and/or 3′ end of the aptamer sequence. For example, the primer handle sequences used in the aptamer may include one or both of the following:
[symbol] (SEQ ID NO:21), a forward primer handle (F-Primer) added to the 5′ end of the aptamer (indicated here and throughout in bold italics)
[symbol] (SEQ ID NO:88), a forward primer handle (F-Primer) added to the 5′ end of the aptamer (indicated here and throughout in bold italics)
TCGGACCTGCACGGC (SEQ ID NO:22), a reverse primer handle (R-Primer) added to the 3′ end of the aptamer (indicated here and throughout in bold) (note that the R-Primer is added as a reverse complement to the 3′ end).

Non-limiting exemplary aptamer sequences that include an F-Primer (bold italics), a first randomized nucleotide sequence, a backbone (underlined), a second randomized nucleotide sequence, and an R-Primer include (bold):

[symbol]

NNNNNNNNNNNNNNNNCCGACCTTGTGCTTT
GGGAGTGCTGGTCCAAGGGCGTTAANNNNNNNNNNNNNNNNNGCCGTGCA
GGTCCGA

(F-Primer, 16mer randomized nucleotide sequence, shortened 1C aptamer backbone, 17mer randomized nucleotide sequence, and R-Primer; SEQ ID NO:23);

[symbol]

NNNNNNNNNNNNNNNNNNNNNACGCAGCATT
TCATCGGGTCCAAAAGGGGCTGCTCGGGATTGCGGATATGGNNNNNNNNN
NNNNNNNNGCCGTGCAGGTCCGA

(F-Primer, 21mer randomized nucleotide sequence, shortened 1C aptamer backbone, 17mer randomized nucleotide sequence, and R-Primer; SEQ ID NO:20);

[symbol]

NNNNNNNNNNNNNNNNNNNNNACGCAGCATT
TCATCGGGTCCAAAAGGGGCTGCTCGGGATTGCGGATATGGNNNNNNNNN
NNNNGCCGTGCAGGTCCGA

(F-Primer, 21mer randomized nucleotide sequence, shortened 1C aptamer backbone, 13mer randomized nucleotide sequence, and R-Primer; SEQ ID NO:24).

Non-limiting exemplary aptamer sequences that include an F-Primer (bold italics), a first randomized nucleotide sequence, a backbone (underlined), and a second randomized nucleotide sequence (bold), include:

[symbol]

NNNNNNNNNNNNNNNNCCGACCTTGTGCTTT
GGGAGTGCTGGTCCAAGGGCGTTAANNNNNNNNNNNNNNNNN

(F-Primer, 16mer randomized nucleotide sequence, shortened 1C aptamer backbone, and 17mer randomized nucleotide sequence; SEQ ID NO:25);

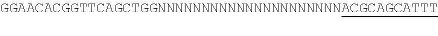

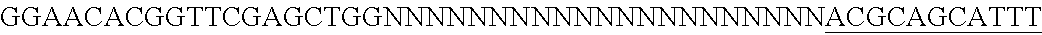

Non-limiting exemplary sequences for an aptamer having an F-Primer (bold italics), a backbone that is wholly composed of a randomized region of nucleotides, and an R-Primer (bold), include:

[symbol]

NNNNNNNNNNNNNNNNNNNNNNNNNNNNNNNN
NNNNNNNNNNNNNNNNNNNNNNNNNNNNNNNNNGCCGTGCAGGTCCGA (F-
primer, 65 mer randomized region, R-Primer; SEQ ID NO: 28) [symbol]
NNNNNNNNNNNNNNNNNNNNNNNNNNNNNNNN
NNNNNNNNNNNNNNNNNNNNNNNNNNNNNNNNGCCGTGCAGGTCCGA (F-
primer, 64 mer randomized region, R-Primer; SEQ ID NO: 29) [symbol]
NNNNNNNNNNNNNNNNNNNNNNNNNNNNNNNN
NNNNNNNNNNNNNNNNNNNNNNNNNNNNNNNGCCGTGCAGGTCCGA (F-
primer, 63 mer randomized region, R-Primer; SEQ ID NO: 30) [symbol]
NNNNNNNNNNNNNNNNNNNNNNNNNNNNNNNN
NNNNNNNNNNNNNNNNNNNNNNNNNNNNNNGCCGTGCAGGTCCGA (F-
primer, 62 mer randomized region, R-Primer; SEQ ID NO: 31) [symbol]
NNNNNNNNNNNNNNNNNNNNNNNNNNNNNNNN
NNNNNNNNNNNNNNNNNNNNNNNNNNNNNGCCGTGCAGGTCCGA (F-primer,
61 mer randomized region, R-Primer; SEQ ID NO: 32) [symbol]
NNNNNNNNNNNNNNNNNNNNNNNNNNNNNNNN
NNNNNNNNNNNNNNNNNNNNNNNNNNNNGCCGTGCAGGTCCGA (F-primer,
60 mer randomized region, R-Primer; SEQ ID NO: 33) [symbol]
NNNNNNNNNNNNNNNNNNNNNNNNNNNNNNNN
NNNNNNNNNNNNNNNNNNNNNNNNNNNGCCGTGCAGGTCCGA (F-primer,
59 mer randomized region, R-Primer; SEQ ID NO: 34).

In some embodiments, the shortened 1C or 4C aptamer backbone is further modified. In some embodiments, the modification is an amino acid substitution where a G is replaced with an A (AG_mut), a deletion of A (Adel), a deletion of G (Gdel), a deletion of A and G (Adel_Gdel), an insertion of A (Ains), or a combination thereof. In other embodiments, the aptamer includes a shortened primer sequence (e.g., N6-D2-NH2).

Non-limiting examples of nucleotide sequences that encode DNA aptamers of a biosensor useful with the present technology and in accordance with the embodiments described herein are provided in Table 1 below. The sequences in Table 1 may include one or more of (i) forward primer handle sequences (F-primer, indicated by bold italic text), and/or (ii) a reverse primer handle sequence (R-primer, indicated by bold text), and/or (iii) a backbone sequence or modified backbone sequence (underlined), and/or (iv) one or more randomized sequences (normal text).

TABLE 1
Nucleotide Sequences

| ID | NAME | SEQUENCE |
|---|---|---|
| N1 | Nar5R12_65c | 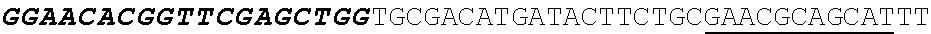 |
| | | CATCGGGTCCAAAAGGGGCTGCTCAGGATTGCGGATATGGACTACGTGCTG |
| | | TGTTTCGCCGTGCAGGICCGA (SEQ ID NO: 35) |
| N2 | Nar5R12_65c_ | 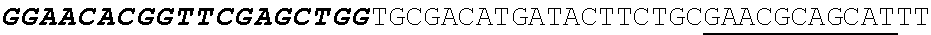 |
| | Adel | 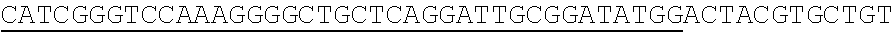 |
| | | GTTTCGCCGTGCAGGTCCGA (SEQ ID NO: 36) |
| N3 | Nar5R12_65c_ | 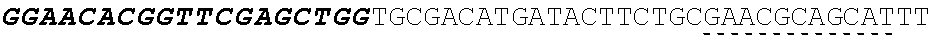 |
| | Gdel | 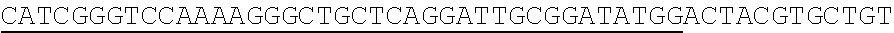 |
| | | GTTTCGCCGTGCAGGTCCGA (SEQ ID NO: 37) |
| N4 | Nar5R12_65c_ | 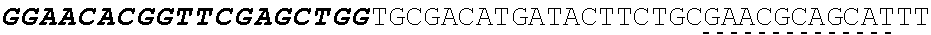 |
| | Adel_Gdel | 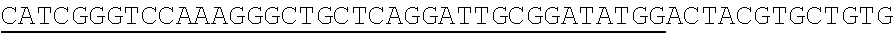 |
| | | TTTCGCCGTGCAGGTCCGA (SEQ ID NO: 38) |
| N5 | Nar5R12_65c_ | 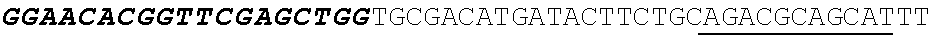 |
| | AG_mut | CATCGGGTCCAAAAGGGGCTGCTCAGGATTGCGGATATGGACTACGTGCTG |
| | | TGTTTCGCCGTGCAGGTCCGA (SEQ ID NO: 39) |
| N6 | Nar5R12_63c | 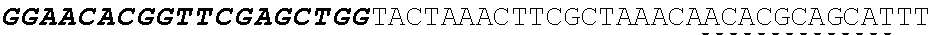 |
| | | 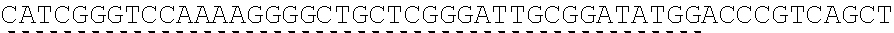 |
| | | TATCGTGCCGTGCAGGTCCGA (SEQ ID NO: 40) |
| N6-D1 | Peter 3′ | 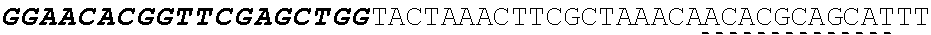 |
| | digested | 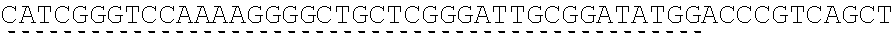 |
| | | TA (SEQ ID NO: 41) |
| N6-D2 | Clem steven | 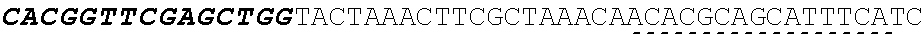 |
| | 3′ digested + | 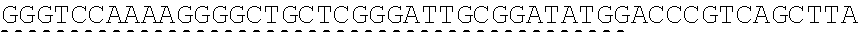 |
| | minus 4 5′ | (SEQ ID NO: 42) |
| N6-D3 | 3′ digested + | 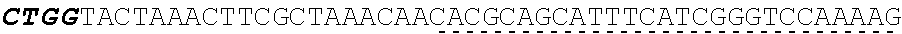 |
| | minus 15 | 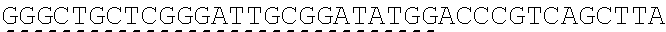 |
| | 5′ | (SEQ ID NO: 89) |
| N6-D4 | 3′ digested + | 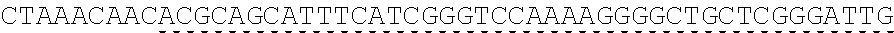 |
| | minus 31 | 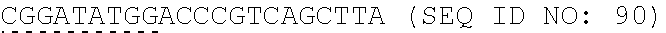 |
| | 5′ | |
| N7 | Nar5R12_63c_ | 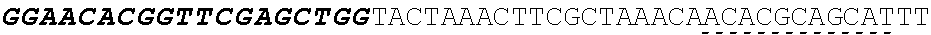 |
| | Adel | 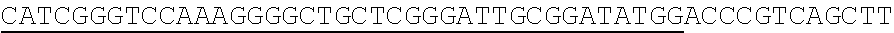 |
| | | ATCGTGCCGTGCAGGTCCGA (SEQ ID NO: 43) |
| N8 | Nar5R12_63c_ | 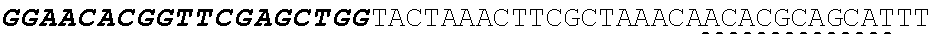 |
| | Gdel | 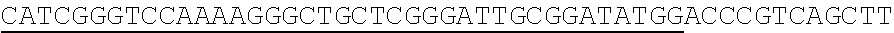 |
| | | ATCGTGCCGTGCAGGTCCGA (SEQ ID NO: 44) |
| N9 | Nar5R12_63c_ | 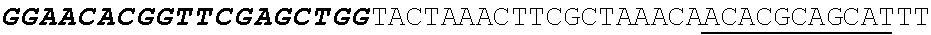 |
| | Ains | CATCGGGTCCAAAAAGGGGCTGCTCGGGATTGCGGATATGGACCCGTCAGC |
| | | TTATCGTGCCGTGCAGGTCCGA (SEQ ID NO: 45) |
| N10 | Nar5R12_63c_ | 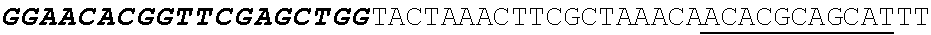 |
| | Adel_Gdel | CATCGGGTCCAAAGGGCTGCTCGGGATTGCGGATATGGACCCGTCAGCTTA |
| | | TCGTGCCGTGCAGGTCCGA (SEQ ID NO: 46) |
| N11 | Nar5R12_60c | 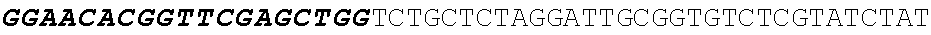 |
| | | GGTTAGTTCCACGTCTGTTGCTTTGTGTGCCGTGCAGGTCCGA (SEQ ID |
| | | NO: 47) |
| N12 | Nar5R12_50c | 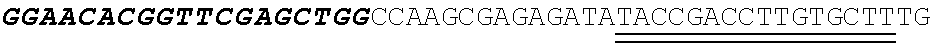 |
| | | 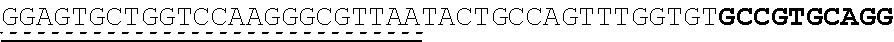 |
| | | TCCGA (SEQ ID NO: 48) |
| N13 | Nar5R12_49c | 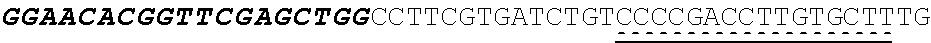 |
| | | 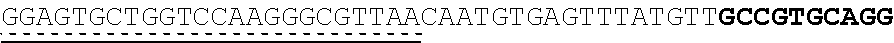 |
| | | TCCGA (SEQ ID NO: 49) |
| N14 | Nar5R12_48c | 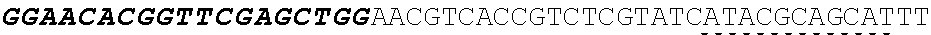 |
| | | 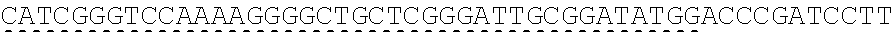 |
| | | CTGTGCGCCGTGCAGGTCCGA (SEQ ID NO: 50) |
| N15 | Nar5R12_45c | 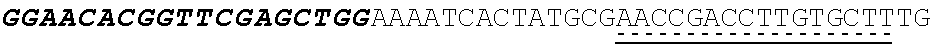 |
| | | 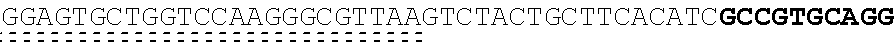 |
| | | TCCGA (SEQ ID NO: 51) |
| N16 | Nar5R12_40c | 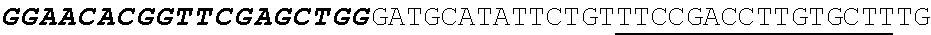 |
| | | 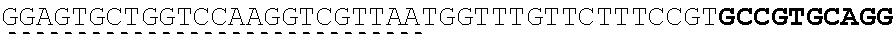 |
| | | TCCGA (SEQ ID NO: 52) |
| N17 | Nar5R12_35c | 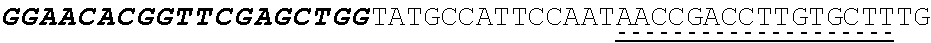 |
| | | 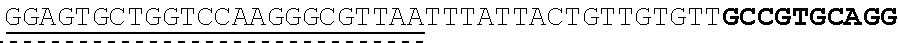 |
| | | TCCGA (SEQ ID NO: 53) |
| NAR18 | NAR5_6381 | 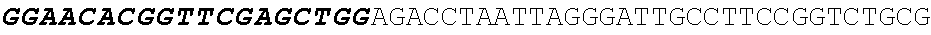 |
| | | GGTGATGTCTGGTTGACAGTTTGGTTTGCCGTGCAGGTCCGA (SEQ ID |
| | | NO: 54) |
| NAR19 | NAR5_179195 | 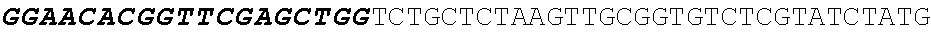 |
| | | GTTAGTTCCACGTCTGTTGCTTTGTGTGCCGTGCAGGTCCGA (SEQ ID |
| | | NO: 55) |
| NAR20 | NAR5_37039 | 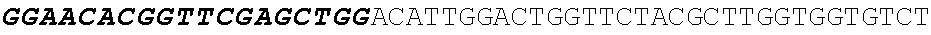 |
| | | TCCTCTATGTTCTTCCGTTGTTTATCCCGCCGTGCCGTGCAGGTCCGA |
| | | (SEQ ID NO: 56) |
| NAR21 | NAR5_29027 | 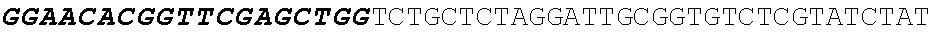 |
| | | GGTTAGTTCCACGTCTGTTGCTTTGTGCCGTGCCGTGCAGGTCCGA (SEQ |
| | | ID NO: 57) |
| NAR22 | NAR5_37033 | 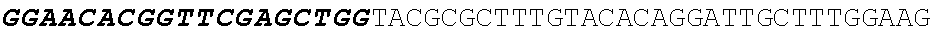 |
| | | TGCTGGTTATCATGTGATTTCTTGTCGTGCCGTGCCGTGCAGGTCCGA |
| | | (SEQ ID NO: 58) |
| NAR23 | NAR5_174521 | 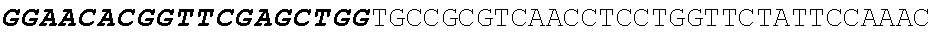 |
| | | TGCTTTGGGAGTGCTGGTCGTTTGGTTTGCCGTGCCGTGCAGGTCCGA |
| | | (SEQ ID NO: 59) |
| NAR24 | NAR5_187972 | 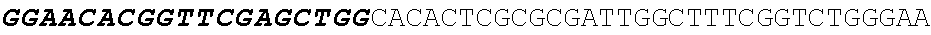 |
| | | AATAGTGTTGTCTGGTTCACCGTATTGTGCCGTGCCGTGCAGGTCCGA |
| | | (SEQ ID NO: 60) |
| NAR25 | NAR5_14769 | 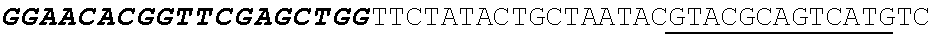 |
| | | 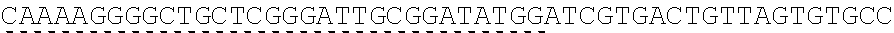 |
| | | GTGCCGTGCAGGTCCGA (SEQ ID NO: 61) |
| NAR26 | NAR5_135036 | 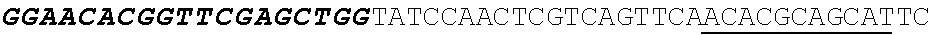 |
| | | 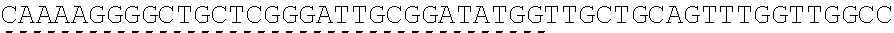 |
| | | GTGCCGTGCAGGTCCGA (SEQ ID NO: 62) |
| NAR27 | NAR5_58918 | 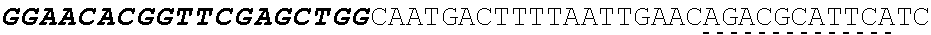 |
| | | 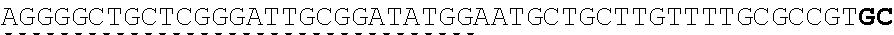 |
| | | CGTGCAGGTCCGA (SEQ ID NO: 63) |
| NAR28 | NAR6 | 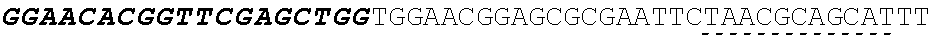 |
| | | 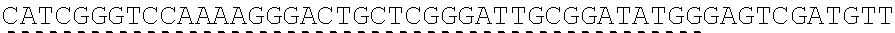 |
| | | TGTCGCGCCGTGCCGTGCAGGTCCGA (SEQ ID NO: 64) |
| NAR29 | NAR6 | 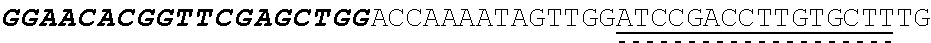 |
| | | 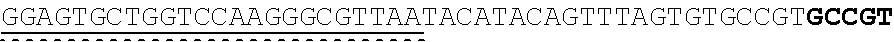 |
| | | GCAGGTCCGA (SEQ ID NO: 65) |
| NAR30 | NAR6 | 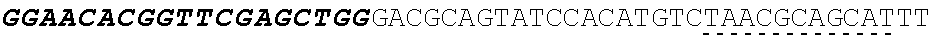 |
| | | 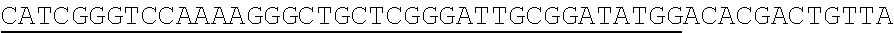 |
| | | ATGTTGCCGTGCCGTGCAGGTCCGA (SEQ ID NO: 66) |
| NAR31 | NAR6 | 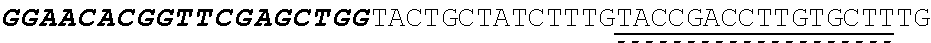 |
| | | 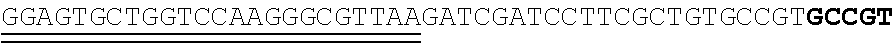 |
| | | GCAGGTCCGA (SEQ ID NO : 67) |
| NAR32 | NAR6 | 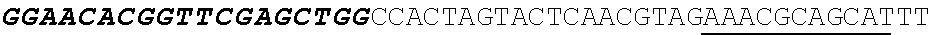 |
| | | CATCGGGTCCAAAGGGCTGCTCGGGATTGCGGATATGGACCCGTTGCTTTA |
| | | TGTTGCCGTGCCGTGCAGGTCCGA (SEQ ID NO: 68) |
| NAR33 | NAR6 | 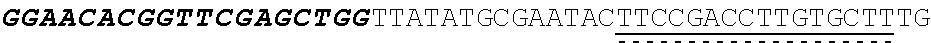 |
| | | 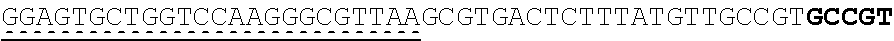 |
| | | GCAGGTCCGA (SEQ ID NO: 69) |
| N34 | NAR5 | 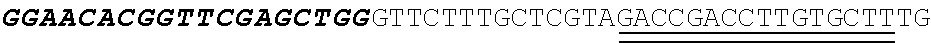 |
| | | 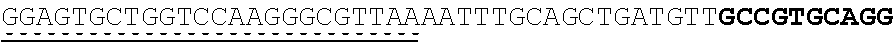 |
| | | TCCGA (SEQ ID NO:70) |
| N35 | NAR5 | 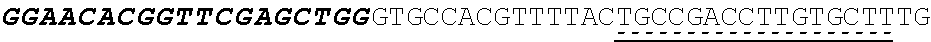 |
| | | 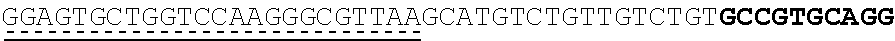 |
| | | TCCGA (SEQ ID NO: 71) |
| N36 | NAR5 | 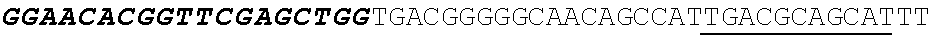 |
| | | CATCGGGTCCAAAAGGGGCTGCTCAGGATTGCGGATATGGACCCGAGACTT |
| | | TACGTTGCCGTGCAGGTCCGA (SEQ ID NO: 72) |
| N37 | NAR5 | 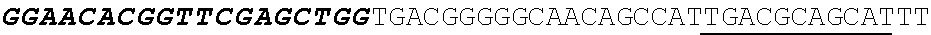 |
| | | CATCGGGTCCAAAAGGGGCTGCTCAGGATTGCGGATATGGACCCGAGACTT |
| | | TACGTTGCCGTGCAGGTCCGA (SEQ ID NO: 73) |
| N38 | NAR5 | 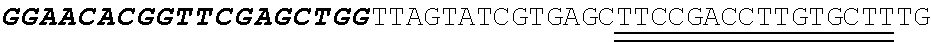 |
| | | 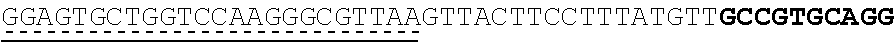 |
| | | TCCGA (SEQ ID NO: 74) |
| N39 | NAR6 | 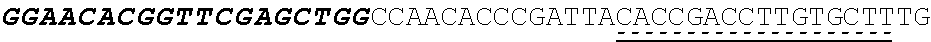 |
| | | 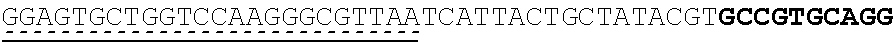 |
| | | TCCGA (SEQ ID NO: 75) |
| N40 | NAR6 | 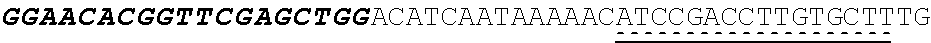 |
| | | 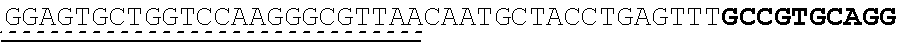 |
| | | TCCGA (SEQ ID NO: 76) |
| N41 | NAR6 | 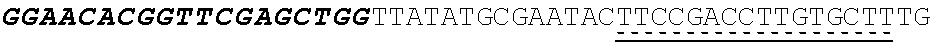 |
| | | 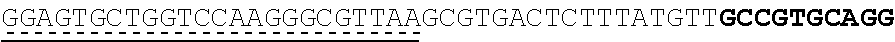 |
| | | TCCGA (SEQ ID NO: 77) |
| N42 | NAR6 | 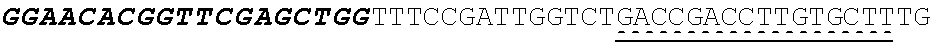 |
| | | 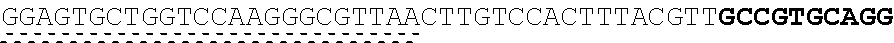 |
| | | TCCGA (SEQ ID NO: 78) |
| N43 | NAR6 | 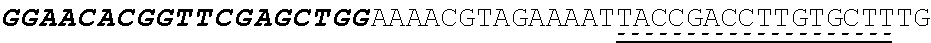 |
| | | 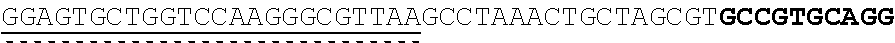 |
| | | TCCGA (SEQ ID NO: 79) |
| N44 | NAR6 | 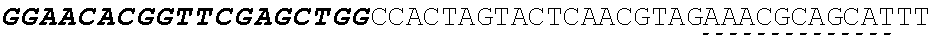 |
| | | 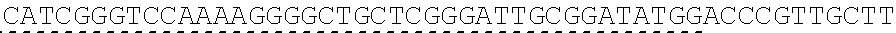 |
| | | TATGTTGCCGTGCAGGTCCGA (SEQ ID NO: 80) |
| N45 | NAR6 | 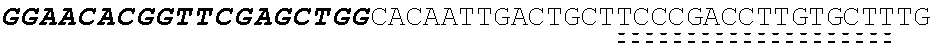 |
| | | 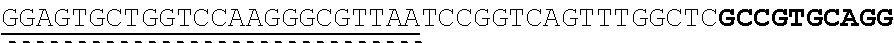 |
| | | TCCGA (SEQ ID NO: 81) |
| N46 | NAR6 | 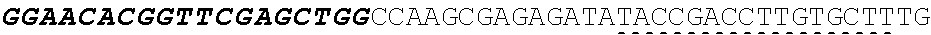 |
| | | 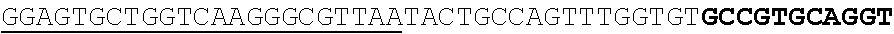 |
| | | CCGA (SEQ ID NO: 82) |
| N47 | NAR6 | 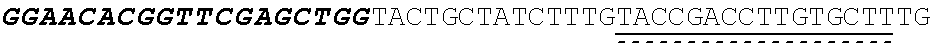 |
| | | 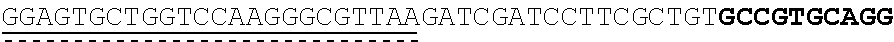 |
| | | TCCGA (SEQ ID NO: 83) |
| N48 | N5R13E1 | 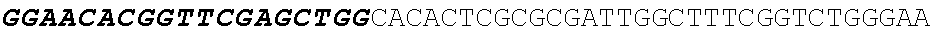 |
| | | AATAGTGTTGTCTGGTTCACCGTATTGTGCCGTGCCGTGCAGGTCCGA |
| | | (SEQ ID NO: 84) |
| N49 | N5R13E2 | 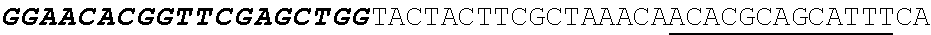 |
| | | TCGGGTCCAAAAAGGGGCTGCTCGGGATTGCGGATATGGACCCGTCAGCTT |
| | | ATCGTGCCGTGCAGGTCCGA (SEQ ID NO: 85) |
| N50 | N5R13E3 | 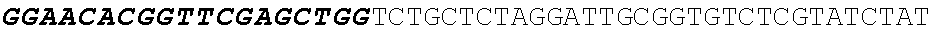 |
| | | TGCGGATATGGAGTGGTGAGTTTATGTTGCCGTGCAGGTCCGA (SEQ ID |
| | | NO: 86) |
| N51 | N5R13E4 | 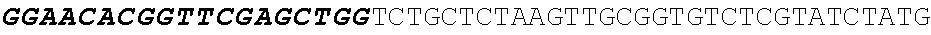 |
| | | GTTAGTTCCACGTCTGTTGCTTTGTGTGCCGTGCAGGTCCGA (SEQ |
| | | ID NO: 87) |

Selection of aptamers may be accomplished by any suitable method known in the art, including SELEX (Systemic Evolution of Ligands by Exponential enrichment). The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), each of which is hereby incorporated by reference in their entirety. An established template-primer system (Bartel et al., “HIV-1 Rev Regulation Involves Recognition of Non-Watson-Crick Base Pairs in Viral RNA,” Cell 67:529-536 (1991), which is hereby incorporated by reference in its entirety) can be adapted to produce oligonucleotides having a stretch of about 20-80 random bases sandwiched between constant regions. Randomized libraries are generated to undergo the SELEX process. Exemplary randomized libraries that can be used to generate candidate aptamers, including those disclosed in Table 1 and throughout the specification, are disclosed in Examples 1 and 2. Once candidate aptamers have been screened for activity, the selected candidates may be further modified or optimized. For example, the 3′ and/or 5′ ends of the aptamer can be digested to shorten the aptamer length. Other examples of optimizations or modifications can include deletions, additions, and substitutions to the candidates. Examples of modified aptamers are found in Table 1 (e.g., N1, N2, N3, N4, N5, N6, N6-D1, N6-D2, N6-D3, N6-D4, N7, N8, N9, N10, N16, NAR28, NAR30, NAR32, NAR36, NAR37, N46, N49).

In certain embodiments, the aptamer has a sequence listed in Table 1. In some embodiments, the sequence of the aptamer is one of the following:

(N1, SEQ ID NO: 35)
GGAACACGGTTCGAGCTGGTGCGACATGATACTTCTGCGAACGCAGCAT
TTCATCGGGTCCAAAAGGGGCTGCTCAGGATTGCGGATATGGACTACGT
GCTGTGTTTCGCCGTGCAGGTCCGA
(N2, SEQ ID NO: 36)
GGAACACGGTTCGAGCTGGTGCGACATGATACTTCTGCGAACGCAGCAT
TTCATCGGGTCCAAAGGGGCTGCTCAGGATTGCGGATATGGACTACGTG
CTGTGTTTCGCCGTGCAGGTCCGA
(N3, SEQ ID NO: 37)
GGAACACGGTTCGAGCTGGTGCGACATGATACTTCTGCGAACGCAGCAT
TTCATCGGGTCCAAAAGGGCTGCTCAGGATTGCGGATATGGACTACGTG
CTGTGTTTCGCCGTGCAGGTCCGA
(N4, SEQ ID NO: 38)
GGAACACGGTTCGAGCTGGTGCGACATGATACTTCTGCGAACGCAGCAT
TTCATCGGGTCCAAAGGGCTGCTCAGGATTGCGGATATGGACTACGTGC
TGTGTTTCGCCGTGCAGGTCCGA
(N5, SEQ ID NO: 39)
GGAACACGGTTCGAGCTGGTGCGACATGATACTTCTGCAGACGCAGCAT
TTCATCGGGTCCAAAAGGGGCTGCTCAGGATTGCGGATATGGACTACGT
GCTGTGTTTCGCCGTGCAGGTCCGA
(N6, SEQ ID NO: 40)
GGAACACGGTTCGAGCTGGTACTAAACTTCGCTAAACAACACGCAGCAT
TTCATCGGGTCCAAAAGGGGCTGCTCGGGATTGCGGATATGGACCCGTC
AGCTTATCGTGCCGTGCAGGTCCGA
(N6-D1, SEQ ID NO: 41)
GGAACACGGTTCGAGCTGGTACTAAACTTCGCTAAACAACACGCAGCAT
TTCATCGGGTCCAAAAGGGGCTGCTCGGGATTGCGGATATGGACCCGTC
AGCTTA
(N6-D2, SEQ ID NO: 42)
CACGGTTCGAGCTGGTACTAAACTTCGCTAAACAACACGCAGCATTTCA
TCGGGTCCAAAAGGGGCTGCTCGGGATTGCGGATATGGACCCGTCAGCT
TA
(N6-D3; SEQ ID NO: 89)
CTGGTACTAAACTTCGCTAAACAACACGCAGCATTTCATCGGGTCCAAA
AGGGGCTGCTCGGGATTGCGGATATGGACCCGTCAGCTTA
(N6-D4, SEQ ID NO: 90)
CTAAACAACACGCAGCATTTCATCGGGTCCAAAAGGGGCTGCTCGGGAT
TGCGGATATGGACCCGTCAGCTTA

In certain embodiments, the aptamers described herein are modified by a functional group that facilitates attachment to the plates used in the assays described herein. In some aspects, the aptamer is functionaled on the 5′ end with an NH2 group or a biotin. Thus, in some embodiments, an aptamer that may be used in accordance with the technology described herein may include the following sequences:

(N6-NH2, SEQ ID NO: 91)
/5AmMC12/GGAACACGGTTCGAGCTGGTACTAAACTTCGCTAAACAAC
ACGCAGCATTTCATCGGGTCCAAAAGGGGCTGCTCGGGATTGCGGATAT
GGACCCGTCAGCTTATCGTGCCGTGCAGGTCCGA
(N6-biotin, SEQ ID NO: 92)
/5BiosG/GGAACACGGTTCGAGCTGGTACTAAACTTCGCTAAACAACA
CGCAGCATTTCATCGGGTCCAAAAGGGGCTGCTCGGGATTGCGGATATG
GACCCGTCAGCTTATCGTGCCGTGCAGGTCCGA
(N6-D1-NH2, SEQ ID NO: 93)
/5AmMC12/GGAACACGGTTCGAGCTGGTACTAAACTTCGCTAAACAAC
ACGCAGCATTTCATCGGGTCCAAAAGGGGCTGCTCGGGATTGCGGATAT
GGACCCGTCAGCTTA
(N6-D1-biotin, SEQ ID NO: 94)
/5BiosG/GGAACACGGTTCGAGCTGGTACTAAACTTCGCTAAACAACA
CGCAGCATTTCATCGGGTCCAAAAGGGGCTGCTCGGGATTGCGGATATG
GACCCGTCAGCTTA
(N6-D2-NH2; SEQ ID NO: 95)
/5AmMC12/CACGGTTCGAGCTGGTACTAAACTTCGCTAAACAACACGC
AGCATTTCATCGGGTCCAAAAGGGGCTGCTCGGGATTGCGGATATGGAC
CCGTCAGCTTA
(N6-D2-biotin; SEQ ID NO: 96)
/5BiosG/CACGGTTCGAGCTGGTACTAAACTTCGCTAAACAACACGCA
GCATTTCATCGGGTCCAAAAGGGGCTGCTCGGGATTGCGGATATGGACC
CGTCAGCTTA
(N6-D3-NH2; SEQ ID NO: 97)
/5AmMC12/CTGGTACTAAACTTCGCTAAACAACACGCAGCATTTCATC
GGGTCCAAAAGGGGCTGCTCGGGATTGCGGATATGGACCCGTCAGCTTA
(N6-D3-biotin; SEQ ID NO: 98)
/5BiosG/CTGGTACTAAACTTCGCTAAACAACACGCAGCATTTCATCG
GGTCCAAAAGGGGCTGCTCGGGATTGCGGATATGGACCCGTCAGCTTA
(N6-D4-NH2; SEQ ID NO: 99)
/5AmMC12/CTAAACAACACGCAGCATTTCATCGGGTCCAAAAGGGGCT
GCTCGGGATTGCGGATATGGACCCGTCAGCTTA
(N6-D4-biotin; SEQ ID NO: 100)
/5BiosG/CTAAACAACACGCAGCATTTCATCGGGTCCAAAAGGGGCTG
CTCGGGATTGCGGATATGGACCCGTCAGCTTA

### Targets

The target can be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. Complexes of two or more molecules can be targets and include, without limitation, complexes have the following interactions: protein-protein, protein-cofactor, protein-inhibiting small molecules, protein-activating small molecules, protein-small molecules, protein-ion, protein-RNA, protein-DNA, DNA-, RNA-DNA, RNA-RNA, modified nucleic acids-DNA or RNA, aptamer-. In addition, targets that possess a mutation can be distinguished from wildtype forms of the target. In some embodiments, the target is associated with an analyte in a sample. Additional targets are described in detail below.

In some embodiments, the target is a nucleic acid and binds specifically to the target domain via hybridization (e.g., Watson-Crick base-pairing). The target domain of the aptamer includes a nucleotide sequence that is sufficiently complementary to its target so as to hybridize under appropriate conditions with the target nucleic acid in the sample. Nucleic acid targets can be any type of nucleic acid including, without limitation, DNA, RNA, LNA, PNA, UNA, genomic DNA, viral DNA, synthetic DNA, DNA with modified bases or backbone, mRNA, noncoding RNA, PIWI RNA, termini-associated RNA, promoter-associated RNA, tRNA, rRNA, microRNA, siRNA, post-transcriptionally modified RNA, synthetic RNA, RNA with modified bases or backbone, viral RNA, bacteria RNA, RNA aptamers, DNA aptamers, ribozymes, and DNAzymes.

In other embodiments, the target is a peptide of any length, including without limitation, phosphoproteins, lipid-modified proteins, nitrosylated proteins, sulfenated proteins, acylated proteins, methylated proteins, demethylated proteins, C-terminal amidated proteins, biotinylated proteins, formylated proteins, gamma-carboxylated proteins, glutamylated proteins, glycylated proteins, iodinated proteins, hydroxylated proteins, isoprenylated proteins, lipoylated proteins (including prenylation, myristoylation, famesylation, palmitoylation, or geranylation), proteins covalently linked to nucleotides such as ADP ribose (ADP-ribosylated) or flavin, oxidated proteins, proteins modified with phosphatidylinositol groups, proteins modified with pyroglutamate, sulfated proteins, selenoylated proteins, proteins covalently linked to another protein (including sumoylation, neddylation, ubiquitination, or ISGylation), citrullinated proteins, deamidated proteins, eliminylated proteins, disulfide bridged proteins, proteolytically cleaved proteins, proteins in which proline residues have been racemized, any peptides sequences that undergo the above mentioned modifications, and proteins which undergo one or more conformational changes. In addition, peptides having a mutation can be distinguished from wildtype forms.

Lipid targets include, without limitation, phospholipids, glycolipids, mono-, di-, tri-glycerides, sterols, fatty acyl lipids, glycerolipids, glycerophospholipids, sphingolipids, sterol lipids, prenol lipids, saccharolipids, polyketides, eicosanoids, prostaglandins, leukotrienes, thromboxanes, N-acyl ethanolamine lipids, cannabinoids, anandamides, terpenes, and lipopolysaccharides.

Small molecule targets include, without limitation, carbohydrates, monosaccharides, polysaccharides, galactose, fructose, glucose, amino acids, peptides, nucleic acids, nucleotides, nucleosides, cyclic nucleotides, polynucleotides, vitamins, drugs, inhibitors, single atom ions (such as magnesium, potassium, sodium, zinc, cobalt, lead, cadmium, etc.), multiple atom ions (such as phosphate), radicals (such as oxygen or hydrogen peroxide), and carbon-based gases (carbon dioxide, carbon monoxide, etc.).

Targets can also be whole cells or molecules expressed on the surface of whole cells. Exemplary cells include, without limitation, cancer cells, bacterial cells, or normal cells. Targets can also be viral particles.

### Linkers

The terms “linker” and “linker domain” are used interchangeably herein. The linker domain is positioned between the reporter domain and target domain. The linker may be about 16 nucleotides or less (for example 8 nucleotides per side), such as about 20 nucleotides or less, about 30 nucleotides or less, or about 40 nucleotides or less. In some embodiments, the linker domain is between about 2 and about 14 nucleotides, between about 4 and about 12 nucleotides, or between about 6 and about 10 nucleotides. The linker domain length may be determined by the needs of the targeting domain. In some embodiments, the linker is symmetrical with half of the linker one either side of the target domain. For example, a linker domain having 16 nucleotides would have two oligonucleotides of eight bases, one on either side flanking the target domain. In other embodiments, the linker domain is asymmetrical with different numbers of nucleotides on each side of the stem.

Linker domain sequences can be generated randomly or selected. Randomly generated linker sequences can be prepared and identified using a library. In some embodiments, libraries of various linker sequences of 4ⁿ can be prepared, where n is the number of nucleotides, unique sequences because the two sides of the linker domain may contain mismatches. These libraries are abbreviated as Nn libraries, where n is the number of nucleotides used in the construction of the library. If using Nn libraries of sufficient size, care should be taken to ensure necessary motifs are not duplicated between the linker domain and target domain.

One or more target domains in the biosensor are attached through one or more linker domains to the reporter domain. Together, the linker domain and the target domain form an additional stem on the reporter domain. Identifying suitable target domain comprising of a polynucleotide involves selecting polynucleotides that bind a particular target molecule with sufficiently high affinity (e.g., K_{d}≤500 nM when not reduced by the linker domain) and specificity from a pool or library of nucleic acids containing a random region of varying or predetermined length.

One or more target domains may be attached to each linker domain. If more than one target domain is used, they may either bind to the same or different targets. Multiple target domains may work independently or together to effect an allosteric change in the biosensor.

The linker domain may or may not be positioned in such a way as to alter the binding of either reporter or target domain for their respective targets. The linker domain may be attached to either the reporter domain or the target domain in such a way to partially destabilize either binding pockets, but without destroying the binding pocket ability. For example, the linker may be coupled to a truncated target domain with a single base pair on the end of the conserved binding pocket in order to partially reduce the binding of the target domain to its target.

### Reporters

Reporters useful with biosensors of the present technology include reporters which emit a signal upon binding to the reporter domain of the aptamer. Non-limiting examples of reporters include luminescent molecules, chemiluminescent molecules, fluorochromes, fluorescent quenching agents, colored molecules, radioisotopes, mass reporters, biotin, avidin, streptavidin, protein A, protein G, antibodies or fragments thereof, polyhistidine, nickel and its ions, Flag tags, myc tags, heavy metals, enzymes, alkaline phosphatase, peroxidase, luciferase, and colorimetric substrates. In some embodiments, the reporter is a fluorochrome. In some embodiments, the fluorochrome has an excitation at a wavelength of 560 nm, 575 nm, and/or 580 nm and an emission wavelength of 596 nm, 600 nm, 602 nm, and/or 610 nm. For example, fluorochromes useful with biosensors of the present technology include rhodamine fluorochromes and variants thereof. Non-limiting examples include those described by Sunbul and Jaschke and Arora et al. In some embodiments, the rhodamine fluorochrome is a sulforhodamine. Example sulforhodamine compounds include di-nitro-sulforhodamine (SR-DN), such as the SR-DN illustrated in Formula I:

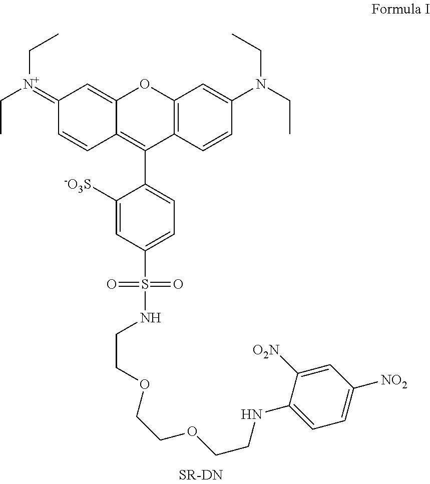

The reporter domain and the fluorophore (e.g., SR-DN) have a low dissociation constant, K_{d}, with the fluorophores. The K_{d} is at least about 0.5 μM, at least about 0.7 μM, at least about 1.0 μM, at least about 1.5 μM, or at least about 2.0 μM. The reporter domain has a fluorophore binding affinity of at least about 400 nM, about 300 nM, about 200 nM, about 100 nM, about 50 nM, about 40 nM, about 30 nM, about 20 nM, about 10 nM, about 5 nM, about 1 nM, or about 0.5 nM when the reporter domain is in a fluorophore binding conformation. When the reporter domain is bound to the reporter, biosensors of the present technology have a brightness of at least 7,000 M/cm, 8,000 M/cm, 9,000 M/cm, 10,000 M/cm, or 43,000 M/cm. The bound reporter has a fluorescent lifetime of at least 1 ns, or at least 2 ns, or at least 3 ns, or at least 4 ns or at least 5 ns, or at least 6 ns, or in the range of 1-6 ns, i.e. 1, or 2, or 3, or 4, or 5 or 6 ns.

Biosensors of the present technology are useful with different aptamers and different reporters. For example, different aptamer backbones can be used with the present technology. In these examples, the aptamer backbones can include one or more randomized regions, such as those described herein or similar to those described herein.

### Additional Oligonucleotides

Additional oligonucleotides besides the aptamer and associated random regions described herein may also be included in the biosensor, at any position on the biosensor. In some embodiments, the additional oligonucleotides are linked to the aptamer and can be positioned on either end of the aptamer such that the aptamer retains function in the biosensor. Non-limiting examples of additional oligonucleotides include primer handles (or “handles”), barcodes, and/or promoters.

Example sequences of primer handles include, but are not limited to:
F-Primer (5′) (indicated in bold italics): [symbol] (SEQ ID NO:21) and
R-Primer (3′) (indicated in bold): [symbol] SEQ ID NO:22).

Without intending to be limiting by the foregoing description, additional oligonucleotides can be useful for sequencing the aptamer; generating the aptamer; and/or identifying the aptamer from a pool of aptamers. Examples include:

(N6D2-forward-CGB; SEQ ID NO: 101)
CACGGTTCGAGCTGGTACTAAA
(N6D2-reverse-CGB; SEQ ID NO: 102)
TAAGCTGACGGGTCCATATCC
(N6D2-L1-KT; SEQ ID NO: 103)
ACGGTTCGAGCTGGTACTAA
(N6D2-R1-KT; SEQ ID NO: 104)
ATATCCGCAATCCCGAGCAG
(N6D2-P1-KT; SEQ ID NO: 105)
ACACGCAGCATTTCATCGGGTCCA

### Methods of Preparing Biosensors

Biosensors of the present technology may be prepared using methods known to those of skill in the art using readily available reagents with no undue experimentation. For example, due to their small size, any known method may be used to synthesize the aptamers. By way of nonlimiting example, the aptamer may be produced using any method of synthetic oligonucleotide syntheses, preferably solid-state synthesis; PCR amplification; or produced in a cell by transforming a host cell with an expression vector comprising the aptamer operantly linked to a promoter capable of expressing the aptamer within the host cell.

While the aptamer of the present technology can be synthesized from chemical precursor, they also can be prepared either in vitro or in vivo using recombinant templates or constructs, including transgenes, that encode the aptamers of the present technology. Whether using in vitro transcription or transgenes suitable for expression in vivo, these genetic constructs can be prepared using well known recombinant techniques. In some embodiments, genetic constructs useful with the present technology include a non-naturally occurring DNA molecule having a first region encoding a DNA aptamer molecule of the present technology.

In some embodiments, the constructed DNA molecule encodes an aptamer of the disclosure, which is formed by joining together one piece of DNA encoding a target domain that is specific for a target ligand and a second piece of DNA encoding a receptor domain that binds specifically to a reporter, and a third piece of DNA encoding the linker domain.

In some embodiments, the aptamer may be made in a modular format though preparing an empty construct for preparation of specific domains of the aptamer. Such an empty construct includes a DNA sequence encoding one or more of the reporter, linker, and/or target domain(s), along with one or more regulatory sequences, and a restriction enzyme insertion site that can be used for subsequent insertion of a desired DNA molecule, which may encode the remaining domains. The restriction enzyme insertion site can include one or more enzymatic cleavage sites to facilitate insertion of virtually any DNA coding sequence as desired. The restriction enzyme insertion site is preferably located between the promoter sequence and the aptamer-encoding DNA sequence.

In some embodiments, DNA molecules include single-stranded DNA molecules having an aptamer nucleic acid sequence, a promoter nucleic acid sequence, and at least one handle nucleic acid sequence (5′ in bold italics, 3′ in bold). Non-limiting examples of single-stranded DNA molecules useful with the present technology include 1C with NY₁ and NY₂ and primer handles:

(SEQ ID NO: 23) [symbol]
NNNNNNNNNNNNNNNNCCGACCTTGTGC
TTTGGGAGTGCTGGTCCAAGGGCGTTAANNNNNNNNNNNNNNNNNGCC
GTGCAGGTCCGA

and 4C with NY₃ and NY₂ and primer handles.

(SEQ ID NO: 20)

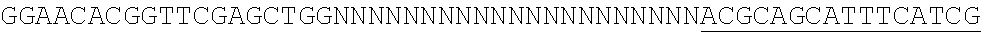

Other single-stranded DNA molecules useful with the present technology include any aptamer sequence found in Table 1 or disclosed throughout this disclosure according to some embodiments.

Once the DNA molecule of the present technology has been constructed, it can be incorporated into cells using conventional recombinant DNA technology. Generally, this involves inserting the DNA molecule into an expression system to which the DNA molecule is heterologous (i.e., not normally present). The heterologous DNA molecule is inserted into the expression system or vector in proper sense orientation. The vector contains the necessary elements for their persistent existence inside cells and for the transcription of an RNA molecule that can be translated into the molecular complex of the present technology.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and transfection and replicated in cultures including prokaryotic organisms and eukaryotic cells grown in tissue culture.

Recombinant viruses can be generated by transfection of plasmids into cells infected with virus. Suitable vectors include, but are not limited to, the following viral vectors such as lambda vector system gtl 1, gt WES.tB, Charon 4, and plasmid vectors such as pBR322, pBR325, pACYCI 77, pACYC184, pUC8, pUC9, pUC18, pUC19, μLG339, pR290, pKC37, pKCIOI, SV 40, pBluescript II SK+/− or KS+/−(see “Stratagene Cloning Systems” Catalog (1993) from Stratagene, La Jolla, Calif), pQE, plH821, pGEX, pET series (see Studier et al., “Use ofT7 RNA Polymerase to Direct Expression of Cloned Genes,” Gene Expression Technology, vol. 185 (1990), pIIIEx426 RPR, pIIIEx426 tRNA (see Good and Engelke, “Yeast Expression Vectors Using RNA Polymerase Ill Promoters,” Gene 151:209-214 (1994), p426GPD (see Mumberg et al., “Yeast Vectors for the Controlled Expression of Heterologous Proteins in Different Genetic Background,” Gene 156:119-122 (1995), p426GAL1 (see Mumberg et al., “Regulatable Promoters of Saccharomyces cerevisiae: Comparison of Transcriptional Activity and Their Use for Heterologous Expression,” Nucl. Acids Res. 22:5767-5768 (1994), pUAST (see Brand and Perrimon, “Targeted Gene Expression as a Means of Altering Cell Fates and Generating Dominant Phenotypes,” Development 118:401-415 (1993), and any derivatives thereof. Suitable vectors are continually being developed and identified.

A variety of host-vector systems may be utilized to express the DNA molecule. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, adeno-associated virus, retrovial vectors, etc.); insect cell systems infected with virus (e.g., baculovirus); and plant cells infected by bacteria or transformed via particle bombardment (i.e., biolistics). The expression elements of these vectors vary in their strength and specificities. Depending upon the host-vector system utilized, any one of a number of suitable transcription elements can be used.

Once the constructed DNA molecule has been cloned into an expression system, it may be incorporated into a host cell. Such incorporation can be carried out by the various forms of transformation, depending upon the vector/host cell system such as transformation, transduction, conjugation, mobilization, or electroporation. The DNA sequences are cloned into the vector using standard cloning procedures in the art, as described by Maniatis et al., Molecular Cloning: A Laboratory Manual, Cold Springs Laboratory, Cold Springs Harbor, New York (1982). Suitable host cells include, but are not limited to, bacteria, yeast, mammalian cells, insect cells, plant cells, and the like. The host cell is preferably present either in a cell culture (ex vivo) or in a whole living organism (in vivo). Mammalian cells suitable for carrying out the present technology include, without limitation, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-1 cells, embryonic stem cells, induced pluripotent stem cells, and primary cells recovered directly from a mammalian organism. With regard to primary cells recovered from a mammalian organism, these cells can optionally be reintroduced into the mammal from which they were harvested or into other animals.

As discussed above, aptamers are generally initially made using randomly generated sequences for both the linker domain and the target domain. Hence, they are initially made in pools of a mixture of different aptamers. Not all the aptamers in this initial pool will have the properties useful for biosensors of the present technology, namely a high affinity for both the reporter and the target ligand and undergo an allosteric shift which effects the signal emitted by the reporter of the biosensor in the presence of the target compared to the absence of the target ligand. Therefore, it is preferable for this initial pool must undergo enrichment selection to reduce the number of possible aptamers.

Any method of selection known in the art may be used to enrich for aptamers which undergo a shift in florescence due to an allosteric shift after a target binds to a target domain. For example, see U.S. patent application Ser. No. 14/235,227 which uses selection in order to enrich for aptamers which bind to both the target and the reporter (e.g., fluorophore), and do not have cross binding to other fluorophores. However, unlike in U.S. patent application Ser. No. 14/235,227 it has been surprisingly found that using alternating rounds of “positive selection” and “negative selection” reduces the number of rounds of selection. As used herein, the term “positive selection” means an enrichment step where the aptamer will bind to the reporter (e.g., fluorophore) in the presence of the target. As used herein, the term “negative selection” means an enrichment step that removes aptamers that bind the fluorophore in the absence of the target.

To perform selection, the reporter (e.g., fluorophore) is bound to a solid substrate. This solid substrate may be any substrate known in the art, including, but not limited to, agarose beads, glass slides, or magnetic beads. The aptamers are then introduced to the bound fluorophores with either the target present or absent. For negative selection, the target is omitted from the process, but for positive selection the target is present. The mixture is incubated to allow time for allosteric binding, followed by washing. The elute resulting from negative selection contains aptamers which may bind the fluorophore only in the presence of the target or may not have an affinity for the fluorophore, therefore some of the aptamers in the elute may be suitable aptamers. The bound aptamers bind to the fluorophore without the target present and are therefore unsuitable as aptamers. The elute from the positive selection are the aptamers which will not bind to the fluorophore in the presence of the target and are therefore unsuitable for use in a biosensor of the present technology. The aptamers which bind the fluorophore in the presence of the target may be suitable as a biosensor, which can then be washed off the solid support. Therefore, the eluate of the negative selection and the bound aptamers in the positive selection may be suitable for use as a biosensor of the present technology.

Any combination of negative and positive selection may be performed. Preferably positive selection follows a round of negative selection to take advantage of the elute of a round of negative selection comprising of aptamers which may be suitable according to this disclosure. Preferably, about 10 or fewer, about 8 or fewer, about 6 or fewer, or about 4 or fewer total rounds of selection are performed. For example, a pool of potential aptamers would be put through a round of negative selection, followed by two alternating rounds of positive followed by negative selection. Following the rounds of enrichment selection, the resulting pool of aptamers may then be optionally sequenced and individual aptamers chosen.

Optionally, it has been surprisingly found that the number of rounds of selection may be minimized by comparing the changes in counts or fold changes between rounds of selection. For example, a pool of potential aptamers, A₀, may undergo a round of negative selection followed by a round of positive selection, pool A₁. Representative samples of A₀ and A₁ may then be sequenced and the counts of unique sequences (potential aptamers) normalized and compared. Aptamers may be selected that exhibit an increase in count from A₀ to A₁ or that have a high A₁:A₀ ratio as these may show allosteric fluorescence.

Another optional selection method would be to split a pool of potential aptamers, B₀, into two equal molar pools, B₀ₐ and B_{0b}. B₀ₐ may then undergo negative selection, B₁ₐ, and B_{0b} may undergo positive selection, B_{1b}. Representative samples of B₀, and the eluate of B₁ₐ, and B_{1b} may then be sequenced and the fold change of B₁ₐ and B_{1b} calculated based on B₀. Potential aptamers may then be selected based on the change in fold change, with some aptamers expected to dim in the presence of the target ligand while others would show allosteric fluorescence.

### Methods of Using Biosensors

Biosensors can be used as detection reagents to determine whether a target is present in a sample. Exemplary targets include pathogens, small molecules, solvents, and ions. The sample may be environmental, such as a water or soil sample, or be isolated from a subject, such as a human or animal blood or tissue sample. One skilled in the art would know how to obtain a sample for use with a biosensor of the present technology. Any of the exemplary targets can be detected individually, e.g., as a detection mechanism for a single target, or combined into a panel including more than two targets.

After the respective sample is obtained, a biosensor of the disclosure is introduced into the sample. The method optionally includes fixing a sample prior to introducing the biosensor, for example, to locate a position of a target within the sample, such as, but not limited to, subcellular structures, RNA, or cells, such as bacteria cells within an environmental sample. The sample, biosensor, and any additional reagents necessary for target binding and/or reporter function are combined and incubated for a period of time until the target binds the target domain. Upon binding of the reporter to the aptamer, a molecular complex between the biosensor and the target is formed.

Molecular complexes of the present disclosure can exist in vitro, in isolated form, or in vivo following introduction of the biosensor (or a genetic construction or expression system encoding the same) into a sample, such as, but not limited to, a host cell or isolated environment or subject sample. In some embodiments, the molecular complex includes at least the aptamer and the reporter bound to the reporter domain of the aptamer (collectively the biosensor), and one or more targets (bound specifically by the target domain(s)). These molecular complexes can exist in vitro, in isolated form or tethered to a substrate such as on an arrayed surface, or in vivo following introduction of the nucleic acid molecule (or a genetic construction or expression system encoding the same) into a host cell.

### Pathogens

Biosensors of the present disclosure are useful for detecting the presence of pathogens within a sample. Non-limiting examples of pathogens include bacterial, viral, prokaryotic, and fungal pathogens, or any non-naturally occurring biologic molecule in a host organism. Exemplary pathogens include, but are not limited to, adenovirus, coronavirus (e.g., HKU1, NL63, 229E, and OC43), human metapneumovirus, human rhinovirus/enterovirus, influenza (e.g., A, A/H1, A/H1-2009, A/H3, and B), parainfluenza (e.g., 1, 2, 3, and 4), respiratory syncytial virus, bordatella pertussis, chlamydophia penumoniae, SARS-CoV, SARS-CoV2 (e.g., COVID-19), MERS-CoV, UPEC, E. coli, Klebsiella pneumoniae, Proteus mirabilis, Pseudomonas aeruginosa, Staphylococcus saprophyticus, Enterococcus faecalis, Enterococcus faecim, Clostridioides difficile, methicillin-resistant Staphylococcus aureus, proteins synthesized by antibiotic resistant bacteria, West Nile virus, Zika virus, Ebola virus, salmonella, equine herpesvirus type I (EHV-1) and type IV (EHV-4), human immunodeficiency virus (HIV), hepatitis A, hepatitis B, hepatitis C, malaria, Dengue virus (DENV-1, -2, -3, -4, and -5), norovirus, rotavirus, astrovirus, Marburg virus, rabies, small pox, measles, and hantavirus. For example, the presence of SARS-CoV2 components, such as proteins (e.g., spike proteins), or portions thereof, can be detected within this sample. The detection of one or more SARS-CoV2 components in a sample can correlate to the presence of the SARS-CoV2 pathogen in the sample thereby detecting the presence of the SARS-CoV2 pathogen in the sample.

At least two or more of the foregoing exemplary pathogens can be combined into a panel, such as a respiratory panel or a urinary tract infection (UTI) panel. An exemplary respiratory panel includes adenovirus, coronavirus (e.g., HKU1, NL63, 229E, and OC43), human metapneumovirus, human rhinovirus/enterovirus, influenza (e.g., A, A/H1, A/H1-2009, A/H3, and B), parainfluenza (e.g., 1, 2, 3, and 4), respiratory syncytial virus, bordatella pertussis, chlamydophia penumoniae, SARS-CoV, SARS-CoV2 (e.g., COVID-19), MERS-CoV, or any combination thereof. An exemplary UTI panel includes, UPEC, E. coli, Klebsiella pneumoniae, Proteus mirabilis, Pseudomonas aeruginosa, Staphylococcus saprophyticus, Enterococcus faecalis, enterococcus faecim, or any combination thereof.

### Small Molecules

Biosensors of the present disclosure are useful for detecting the presence of small molecules within a sample. Non-limiting examples of small molecules include toxins and pharmaceutical agents. Exemplary small molecules include, but are not limited to, cannabinoids (e.g., cannabidiol, cannabinol, and tetrahydrocannabinol), bisphenol A, fluoride, and benzene.

### Solvents

Biosensors of the present disclosure are useful for detecting the presence of solvents within a sample. Exemplary solvents include, but are not limited to, acetone, cyclohexane, acetic acid, ethanol, and benzene.

### Ions

Biosensors of the present disclosure are useful for detecting the presence of ions within a sample. Exemplary ions include, but are not limited to, potassium, chloride, sodium, lithium, magnesium, mercury, and lead.

Detection of Target

Molecular complexes of the present disclosure can be exposed to an appropriate wavelength(s) of energy to activate the reporter (e.g., excitation wavelength) which emits the signal a different wavelength emitted (e.g., emission wavelength). The signal emitted by the reporter can be qualified or quantified based on a difference in brightness to determine if the target was present in the sample. For example, the difference in brightness of the biosensor in the sample to a control biosensor can be measured and determined. The change in brightness may either be an increase in brightness due to allosteric fluorescence or a dimming in brightness when compared to a control sample. For example, as discussed in the examples below, the emitted fluorescence signal for a SARS-CoV2 spike protein is lower than a fluorescence signal emitted by a control. As another example, the sample could be compared to a control reporter within the sample. A known quantity of the control reporter may be added across samples, allowing the comparison of signal from one sample to another.

In some embodiments, molecular complexes of the present disclosure can be identified, quantified, and monitored. Detection of molecular complex formation, through the fluorescent output of the fluorophore, a FRET partner (e.g., donor or acceptor), or a partner similar to a FRET partner, can be used to detect complex formation in a cell-free sample (e.g., cell extracts, fractions of cell extracts, or cell lysates), histological or fixed samples, tissues or tissue extracts, bodily fluids, serum, blood and blood products, environmental samples, or in whole cells. Thus, detection and quantification can be carried out in vivo by fluorescence microscopy or the like, or detection and quantification can be carried in vitro on any of the above extracts or on a sample obtained via in vitro mixing of sample materials and reagents.

Regardless of the intended use, a suitable radiation source is used to illuminate the fluorophore after exposing the fluorophore and aptamer to one another. The radiation source can be used alone or with optical fibers and any optical waveguide to illuminate the sample. Suitable radiation sources include, without limitation, filtered, wide-spectrum light sources (e.g., tungsten, or xenon arc), laser light sources, such as gas lasers, solid state crystal lasers, semiconductor diode lasers (including multiple quantum well, distributed feedback, and vertical cavity surface emitting lasers), dye lasers, metallic vapor lasers, free electron lasers, and lasers using any other substance as a gain medium. Common gas lasers include Argon-ion, Krypton-ion, and mixed gas (e.g., Ar Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 mn. Typical output lines from solid state crystal lasers include 532 nm (doubled Nd:YAG) and 408/816 nm (doubled/primary from Ti:Sapphire). Typical output lines from semiconductor diode lasers are 635, 650, 670, and 780 mm. Infrared radiation sources can also be employed.

In certain embodiments, detection and quantification is carried out by a suitable commercially available plate (or microplate) reader that is capable of wavelength settings or filters that provide efficient excitation of the fluorophore and allow for detectable emission by the fluorophore (e.g., SR-DN). The emission spectra can then be used to compare brightness between samples containing the target vs. samples that do not contain the target. Excitation wavelengths and emission detection wavelengths vary depending on both the fluorophore and the nucleic acid aptamer molecule that are being employed. In certain embodiments, the plate reader filters are set to excitation wavelengths of approximately 575 nm±15 nm, and emission wavelengths of approximately 610 nm±15 nm. In certain embodiments, the plate reader filters are set to excitation wavelengths of approximately 575 nm±10 nm, and emission wavelengths of approximately 610 nm±10 nm. In certain embodiments, the plate reader filters are set to excitation wavelengths of approximately 575 nm±5 nm and emission detection wavelengths of approximately 610 nm±5 nm. In one embodiment, SR-DN has an excitation of 575 nm and an emission of 802 nm.

Detection of the emission spectra can be achieved using any suitable detection system. Exemplary detection systems include, without limitation, a cooled CCD camera, a cooled intensified CCD camera, a single-photon-counting detector (e.g., PMT or APD), dual-photon counting detector, spectrometer, fluorescence activated cell sorting (FACS) systems, fluorescence plate readers, fluorescence resonance energy transfer, and other methods that detect photons released upon fluorescence or other resonance energy transfer excitation of molecules.

In embodiments where the reporters are attached to substrates, such as a glass slide or in microarray format, it is desirable to reject any stray or background light in order to permit the detection of low intensity fluorescence signals. In one embodiment, a small sample volume (about 10 nl) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

In one embodiment, spatial discrimination of a molecular complex of the technology attached to a substrate in a direction normal to the interface of the substrate is obtained by evanescent wave excitation. This is illustrated in PCT Application Publ. No. WO/2010/096584 to Jaffrey and Paige. Evanescent wave excitation utilizes electromagnetic energy that propagates into the lower-index of refraction medium when an electromagnetic wave is totally internally reflected at the interface between higher and lower-refractive index materials. In this embodiment a collimated laser beam is incident on the substrate/solution interface at an angle greater than the critical angle for total internal reflection (TIR). This can be accomplished by directing light into a suitably shaped prism or an optical fiber. In the case of a prism, the substrate is optically coupled (via index-matching fluid) to the upper surface of the prism, such that TIR occurs at the substrate/solution interface on which the molecular complexes are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the molecular complex, whether a change in intensity or polarization. Spatial discrimination in the plane of the interface (i.e., laterally) is achieved by the optical system.

In the embodiment described above, a TIRF evanescent wave excitation optical configuration is implemented using a detection system that includes a universal fluorescence microscope. Any fluorescent microscope compatible with TIRF can be employed. The TIRF excitation light or laser can be set at either an angle above the sample shining down on the sample, or at an angle through the objective shining up at the sample. Effective results can be obtained with immobilization of either the aptamer or the fluorophore using NHS-activated glass slides. The fluorophore containing a free amine (at the R1 position) can be used to react with the NHS-slide. RNA can be modified with a 5′ amine for NHS reactions by carrying out T7 synthesis in the presence of an amine modified GTP analog (commercially available).

In the several embodiments described above, the output of the detection system is preferably coupled to a processor for processing optical signals detected by the detector. The processor can be in the form of personal computer, which contains an input/output (I/O) card coupled through a data bus into the processor. CPU/processor receives and processes the digital output signal and can be coupled to a memory for storage of detected output signals. The memory can be a random-access memory (RAM) and/or read only memory (ROM), along with other conventional integrated circuits used on a single board computer as are well known to those of ordinary skill in the art. Alternatively, or in addition, the memory may include a floppy disk, a hard disk, CD ROM, or other computer readable medium which is read from and/or written to by a magnetic, optical, or other reading and/or writing system that is coupled to one or more processors. The memory can include instructions written in a software package (for image processing) for carrying out one or more aspects of the present technology as described herein.

### Kits

Kits of the present disclosure include, but are not limited to, kits having components useful for determining presence of a target in a sample. In some embodiments, the kit comprises the biosensor, such as the aptamer, the reporter (e.g., SR-DN), reference dye, buffers, solvents, additional nucleic acids, and/or instructions for use.

In accordance with some embodiments, a kit or system may include one or more plates containing a plurality of wells having a covalently or otherwise attached aptamer (or probe) for the detection of a target antigen (i.e., “assay plates”), According to certain embodiments, an aptamer (e.g., those provided in Table 1 above and those disclosed herein) included as part of a biosensor can include an end modifier that allows the aptamer to be immobilized or tethered on a plate for analysis. In one embodiment, an aptamer may be immobilized in accordance with the working examples below. In an another embodiment, an aptamer (e.g., those provided in Table 1 above and those disclosed herein) may have an amino linker C12 modification at the 5′ end of the DNA sequence (“5AmMC12”) that allows for conjugation of the aptamer to a COOH-coated plate. In another embodiment, an aptamer (e.g., those provided in Table 1 above and those disclosed herein) may be modified to include a biotin modification at the 5′ end of the DNA sequence that allows for conjugation of the aptamer to a streptavidin-coated plate. In other embodiments, any suitable end modifier may be used to conjugate the aptamer to plate coated with a moiety to which the modifier can be covalently bound. In one embodiment, such a plate with one or more covalently bound or otherwise attached aptamers (or probes) thereto may include a microplate containing wells having a covalently attached aptamer (or probe) for the detection of CoV-2. In some embodiments, the microplate may include 6, 9, 12, 16, 24, 36, 96, 384, or 1536 wells.

According to the embodiments described herein, a kit or system may also include one or more of a buffer, a ready to use solution containing a reporter, and an excel (or other data analysis tool) template for analyzing the results of an assay performed using the kit or system. In some embodiments a kit or system includes one, two, three, four, five, six, seven, eight, nine, ten, eleven, twelve, thirteen, fourteen, fifteen, sixteen, seventeen, eighteen, nineteen, twenty, or more than twenty assay plates. In one embodiment, the kit or system includes five assay plates. In another embodiment, the kit or system includes ten assay plates. In some embodiments, the buffer included in a kit or system is a salt-based buffer. In some embodiments, the target antigen is a CoV-2 antigen. In certain embodiments, the aptamer(s) bound to the plate is one or more of the aptamers in Table 1 above or any other aptamer described herein. In some embodiments, the reporter is a fluorescent molecule.

In one embodiment, a plate having one or more covalently bound aptamers thereto may be part of a system or kit used to detect a target when a sample is added to and interacts with the biosensor, as discussed in the Examples below. In another embodiment, the system or kit includes one or more of the components described in the Examples below.

The disclosure is further illustrated by the following example which should not be construed as limiting. The examples are illustrative only, and are not intended to limit, in any manner, any of the aspects described herein. The following examples do not in any way limit the technology.

Without further description, it is believed that one of ordinary skill in the art may, using the preceding description and the following illustrative examples, make and utilize the agents of the present disclosure and practice the claimed methods. The following working examples are provided to facilitate the practice of the present disclosure and are not to be construed as limiting in any way the remainder of the disclosure.

### EXAMPLES

### Example 1: 1C Aptamer Backbone for Randomized Libraries (Prophetic)

This example describes the 1C aptamer backbone useful for randomized libraries. The nucleotide sequence of 1C is

(SEQ ID NO: 1)
CAGCACCGACCTTGTGCTTTGGGAGTGCTGGTCCAAGGGCGTTAATGG
ACA.

Randomized regions will be inserted after removing 11 nts from the 1C nucleotide sequence, 5 nts on the 5′ end and 6 nts on the 3′ end (indicated by underlining):

(SEQ ID NO: 1)
CAGCACCGACCTTGTGCTTTGGGAGTGCTGGTCCAAGGGCGTTAATGG
ACA.

and replacing the underlined regions with the following randomized regions NY₁ and

(SEQ ID NO: 3)
NY₁CCGACCTTGTGCTTTGGGAGTGCTGGTCCAAGGGCGTTAANY₂

NY₁ corresponds to 16 nts and NY₂ corresponds to 17 nts. The nucleotide sequence of 1C having both randomized regions will be.

(SEQ ID NO: 18)
NNNNNNNNNNNNNNNNCCGACCTTGTGCTTTGGGAGTGCTGGTCCAAG
GGCGTTAANNNNNNNNNNNNNNNNN.

FIG. 1A illustrates the native 1C aptamer sequence and a location of where the NY₁ and NY₂ positions will be disposed within 1C.

FIGS. 1B and 1C illustrate computer-generated simulations of 1C modified to include the 33 nt random region as well as two primers at 30.0° C. and 20.0° C. Once generated, the 1C aptamer sequence having the 33 nt random region and primer handle will have the nucleotide sequence of:

(SEQ ID NO: 23) [symbol]
NNNNNNNNNNNNNNNNCCGACCTTGTG
CTTTGGGAGTGCTGGTCCAAGGGCGTTAANNNNNNNNNNNNNNNNN
GCCGTGCAGGTCCGA.

The oligonucleotides that will be generated for Example 1 are as follows:
(1) DNA Components:
   (A) Randomized region (33 nts)

(SEQ ID NO: 8)
NNNNNNNNNNNNNNNNNNNNNNNNNNNNNNNNN;
and
(i)
(SEQ ID NO: 14)
NY₁: NNNNNNNNNNNNNNNN
(SEQ ID NO: 13)
NY₂: NNNNNNNNNNNNNNNNN

(B) 1C (aptamer) sequence:

(SEQ ID NO: 1)
CAGCACCGACCTTGTGCTTTGGGAGTGCTGGTCCAAGGGCGTTAATGG
ACA.

(2) DNA Aptamer with Randomized Substitutions:

(SEQ ID NO: 18)
NNNNNNNNNNNNNNNNCCGACCTTGTGCTTTGGGAGTGCTGGTCCAAG
GGCGTTAANNNNNNNNNNNNNNNNN.

(3) Primer handles:

(A)
F-Primer:
(SEQ ID NO: 21)

[symbol]

(B)
R-Primer:
(SEQ ID NO: 22)
TCGGACCTGCACGGC

(4) DNA tamer with Randomized Substitutions and Primer handles:

(SEQ ID NO: 23) [symbol]
NNNNNNNNNNNNNNNNCCGACCTTGTGCTTT
GGGAGTGCTGGTCCAAGGGCGTTAANNNNNNNNNNNNNNNNNGCCGTGCA
GGTCCGA.

### Example 2: 4C Aptamer Backbone for Randomized Libraries (Prophetic)

This example describes the 4C aptamer backbone useful for randomized libraries. The nucleotide sequence of 4C is

(SEQ ID NO: 2)
ATCCAGAGTGACGCAGCATTTCATCGGGTCCAAAAGGGGCTGCTCGGGAT
TGCGGATATGGACACGT.

Randomized regions will be inserted after removing 16 nts from the 4C nucleotide sequence, 10 nts on the 5′ end and 6 nts on the 3′ end (indicated by underlining):

(SEQ ID NO: 2)
ATCCAGAGTGACGCAGCATTTCATCGGGTCCAAAAGGGGCTGCTCGGGAT
TGCGGATATGGACACGT

and replacing the underlined regions with the following randomized regions NY₃ and NY₂:

(SEQ ID NO: 4)
NY₃ACGCAGCATTTCATCGGGTCCAAAAGGGGCTGCTCGGGATTGCGGAT
ATGGNY₂.

NY₃ corresponds to 21 nts and NY₂ corresponds to 17 nts. The nucleotide sequence of 4C having both randomized regions will be:

(SEQ ID NO: 19)
NNNNNNNNNNNNNNNNNNNNNACGCAGCATTTCATCGGGTCCAAAAGGGG
CTGCTCGGGATTGCGGATATGGNNNNNNNNNNNNNNNNN

FIG. 2A illustrates the native 4C aptamer sequence and a location of where the NY₃ and NY₂ positions will be disposed within 4C.

FIG. 2B illustrates computer-generated simulations of 4C modified to include the 38 nt random region as well as two primer handless at 20.0° C. Once generated, the 4C aptamer sequence having the 38 nt random region and primer handles will have the nucleotide sequence of:

(SEQ ID NO: 20) [symbol]
NNNNNNNNNNNNNNNNNNNNNACGCAGCATT
TCATCGGGTCCAAAAGGGGCTGCTCGGGATTGCGGATATGGNNNNNNNNN
NNNNNNNNGCCGTGCAGGTCCGA.

The oligonucleotides that will be generated for Example 1 are as follows:
(1) DNA Components:
   (A) Randomized region (38 nts)

| (SEQ ID NO: 10) | |
|---|---|
| | NNNNNNNNNNNNNNNNNNNNNNNNNNNNNNNNNNNNNN; |
| | and |
| | (i) |
| (SEQ ID NO: 11) | |
| | NY₃: NNNNNNNNNNNNNNNNNNNNN |
| | (ii) |
| (SEQ ID NO: 13) | |
| | NY₂: NNNNNNNNNNNNNNNNN |

(B) 4C (aptamer) sequence:

(SEQ ID NO: 2)
ATCCAGAGTGACGCAGCATTTCATCGGGTCCAAAAGGGGCTGCTCGGGAT
TGCGGATATGGACACGT

(2) DNA Aptamer with Randomized Substitutions:

(SEQ ID NO: 19)
NNNNNNNNNNNNNNNNNNNNNACGCAGCATTTCATCGGGTCCAAAAGGGG
CTGCTCGGGATTGCGGATATGGNNNNNNNNNNNNNNNNN

(3) Primer handles:

| | (A) F-Primer: |
|---|---|
| (SEQ ID NO: 21) | |
| | [symbol] |
| | (B) R-Primer: |
| (SEQ ID NO: 22) | |
| | TCGGACCTGCACGGC |

(4) DNA Aptamer with Randomized Substitutions and Primer Handles:

(SEQ ID NO: 20) [symbol]
NNNNNNNNNNNNNNNNNNNNNACGCAGCATT
TCATCGGGTCCAAAAGGGGCTGCTCGGGATTGCGGATATGGNNNNNNNNN
NNNNNNNNGCCGTGCAGGTCCGA.

### Example 3: Graphene Oxide Enhances Detection of Proteins by DNA Aptamers

This example describes detection of SARS-CoV2 spike proteins by DNA aptamers in the presence and absence of graphene oxide. Compared to control samples, detection of SARS-CoV2 spike proteins by DNA aptamers is more sensitive and accurate in the presence of graphene oxide compared to the absence of graphene oxide.

Methods. At room temperature in ambient light, the following solutions were prepared and combined into various wells of a 96-well white plate, each well at total a volume of 100 μL:
DNA stock: 1 μM
Dye stock DMSO: 2 μL of 500 μM SR-DN in dimethylsulfoxide (DMSO) and 1 mL of 1× binding wash buffer (BWB; 1 M NaCl, 20 mM KCl, 50 mM MgCl₂, and 200 mM Tris.
Dye stock water: 3 mg of SR-DN powder in 2 mL water agitated overnight and filtered.
Spike protein stock solution: 0.039 ng/ml SARS-CoV2 spike protein
Graphene oxide (GO) solution: 50 μL GO stock at 4 mg/ml concentration diluted in 1 mL 1×BWB.

Conditions tested in each well include dye only, dye and SARS-CoV2 spike protein, dye and DNA aptamer, and dye with SARS-CoV2 spike protein and DNA aptamer. Solutions were combined starting with BWB, DNA aptamer, SARS-CoV2 spike protein, and dye.

Once solutions were combined in each well, the plate was exposed to fluorescent light at a wavelength of 575 nm. The fluorophore in the dye, SR-DN, emitted spectra at 610 nm. Following the first exposure, 50 μL of GO solution was added to each well and mixed, and exposed to 575 nm of fluorescent light with SR-DN spectra emitted at 610 nm.

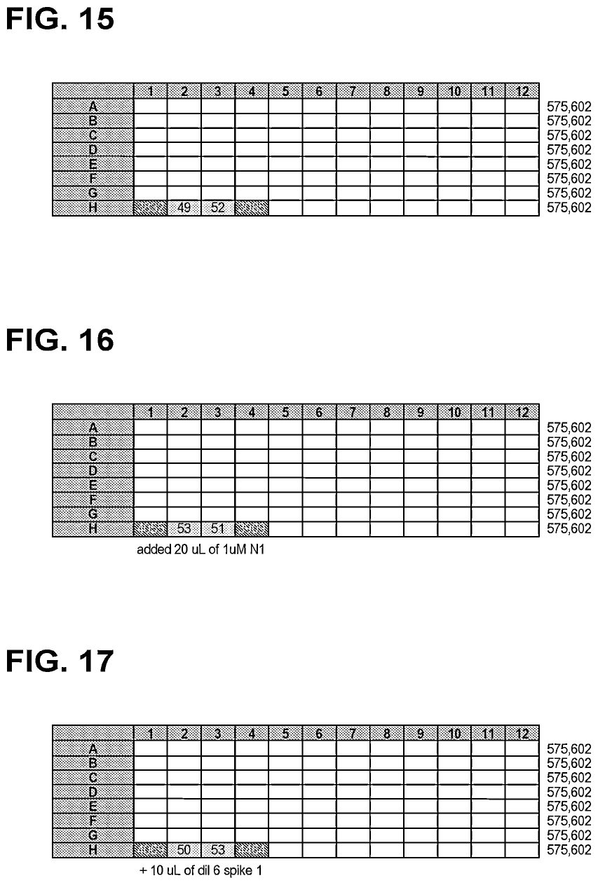

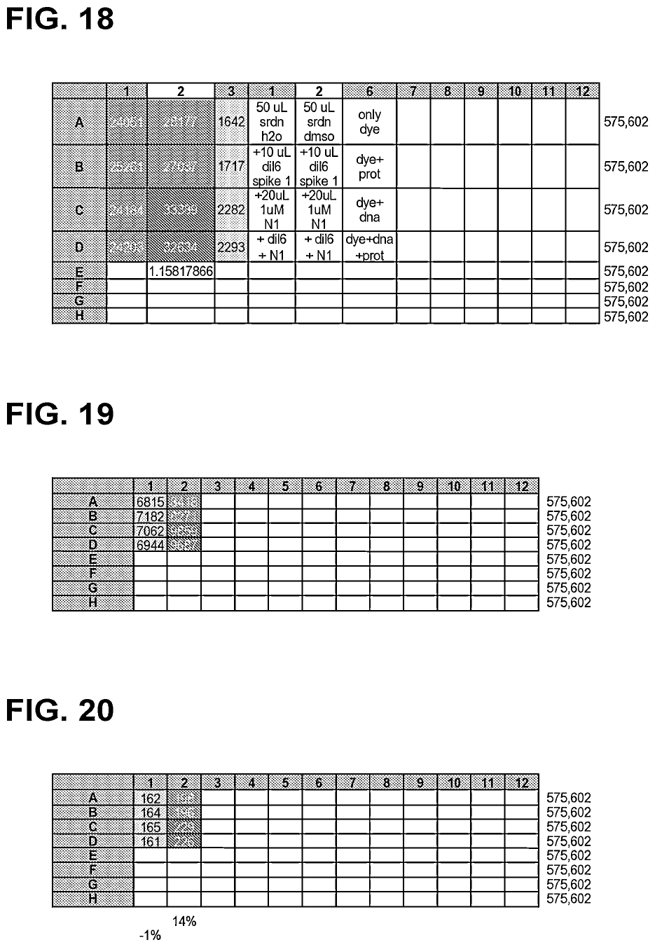

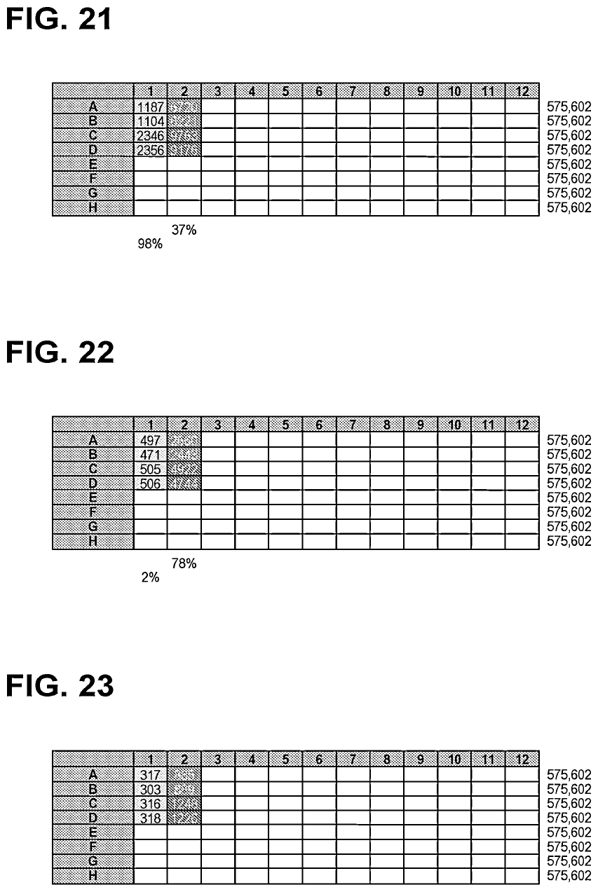

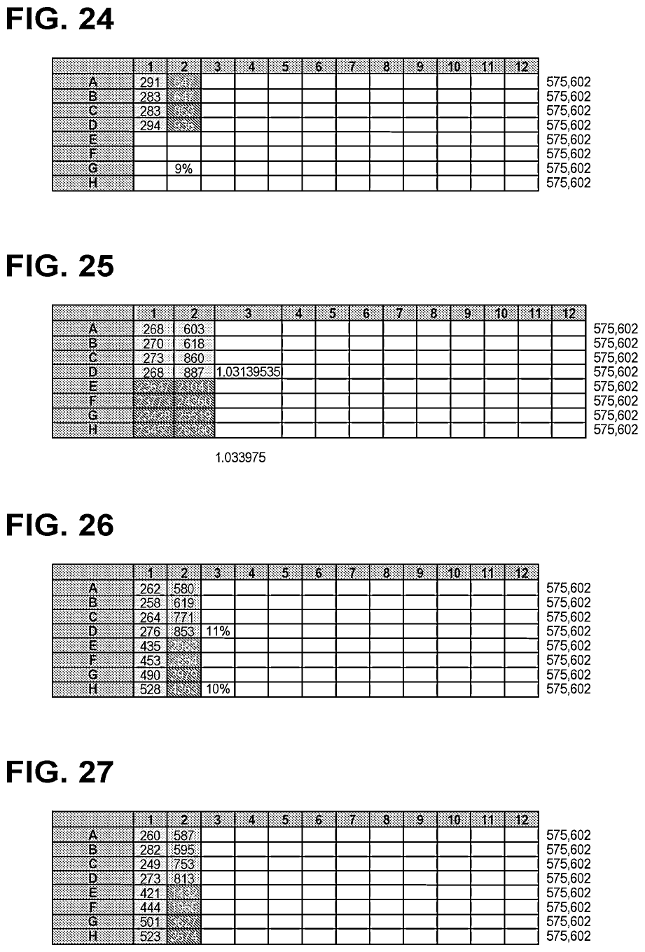

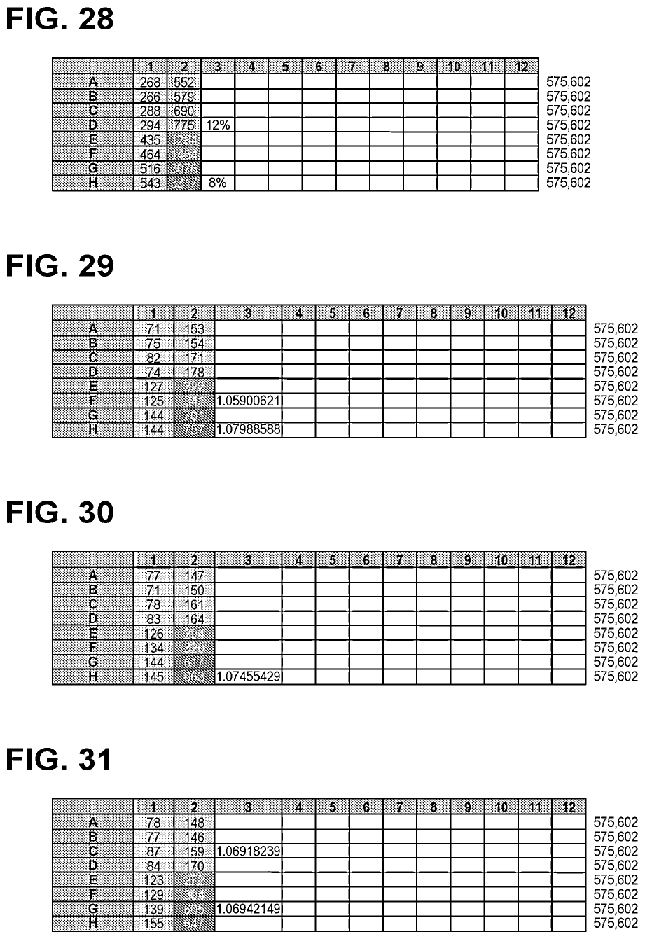

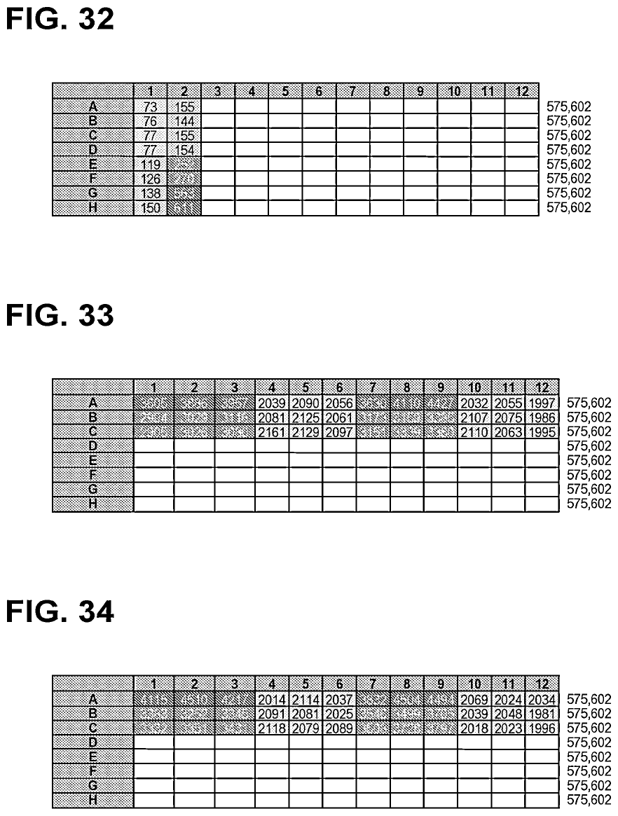

Data and plate maps from multiple experiments using these and generally similar conditions are provided in FIGS. 15-32.

### Example 4: DNA Aptamers Detect Presence of Spike Proteins

This example describes detection of SARS-CoV2 spike proteins (S1 and S2) and MERS spike proteins (MERS) by DNA aptamers. The methods and materials used in Example 4 are generally similar to those of Example 3 with at least the following exceptions. The final concentrations of spike proteins ranged from 0.308 μM to 3.077×10¹¹ μM), the fluorescent dye in a range of about 5 nM to about 50 nM, and the aptamer in a range of about 50 nM to about 500 nM. Compared to control samples, emitted fluorescence decreased for SARS-CoV2 DNA aptamers compared to MERS DNA aptamers which increased in emitted fluorescence compared to control upon detection of the corresponding spike proteins.

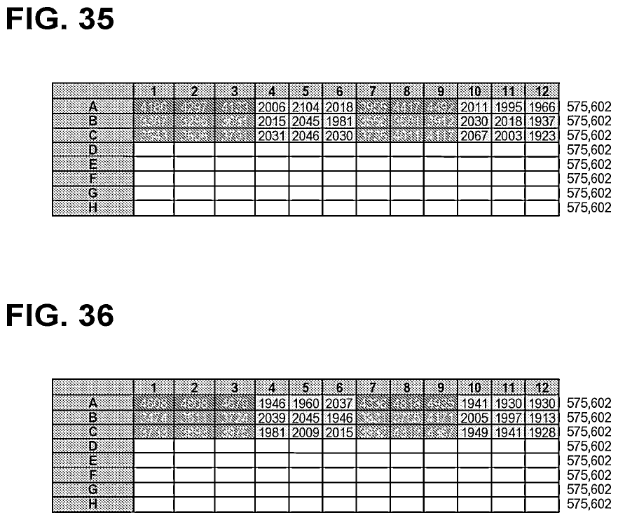

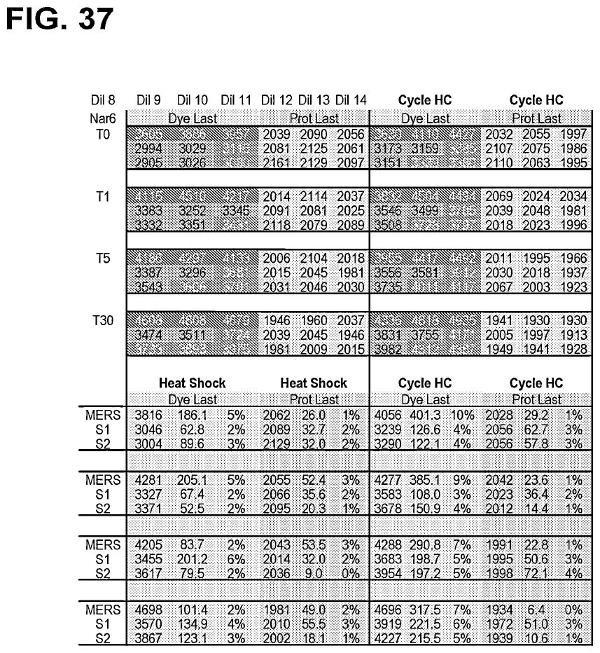

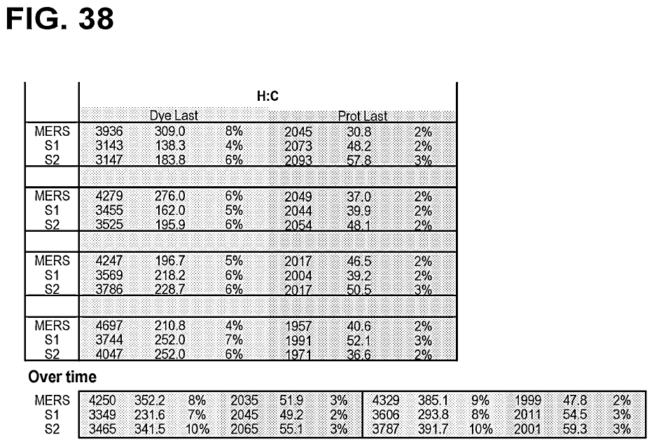

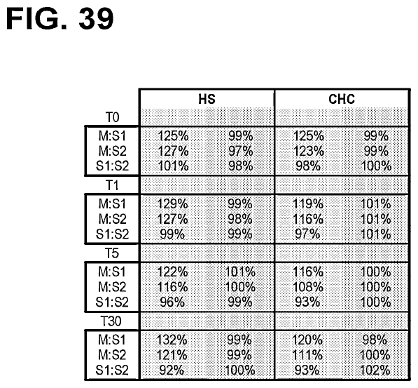

Data and plate maps from multiple experiments using these and generally similar conditions are provided in FIGS. 33-39.

### Example 5: Plate Activation and DNA Aptamer Immobilization

This example describes activation of 96-well plates useful for immobilizing one or more DNA aptamers, such as those described herein. Plates that can be used with the present example include activated plates, such as plates coated with COOH which covalently binds to free amino groups. Alternatively, the plates are coated with streptavidin. Binding to the COOH occurs through formation of amide bonds after exposure to carbodiimide and N-Hydroxysuccinimide (NHS). In the case of streptavidin, binding occurs through formation of a bond with biotin. Thus, aptamers of the present technology are functionaled on the 5′ end with a C11-NH2 group or biotin. A non-limiting example commercial source for COOH-coated plates is Bioworld. Activated plates having immobilized DNA aptamers are useful in accordance with embodiments of the present technology, such as for detection of SARS-CoV-2 spike proteins using the immobilized DNA aptamers. Buffers and DNA aptamer solutions of the following methods are to be prepared before plate activation.

Methods. Buffer Preparation:
Prepare filtered 0.01M 2 ethanesulfonic acid (MES) buffer pH 5.0.
Prepare filtered 300 mM ethanolamine blocking solution (pH 8.5).
Prepare 120 mM 1-ethyl-3-(3-dimethylaminopropyl)carbodiimide (EDC)+30 mM NHS solution in 0.01M MES (this solution is time sensitive, do not use a solution more than a few hours old).

Methods. Aptamer Preparation:
Dilute the 100 μM DNA stock to 10 μM with Nuclease Free Water.
Heat-shock the 10 μM aptamer solution (90° C. for 5 mins+4° C. for 10 mins).
Dilute the heat-shocked 10 μM aptamer solution to 20 nM (710 ng/μL) using the 0.01M MES buffer pH 5.0.

Methods. Plate Activation:
Rinse the COOH-plate 3× with 200 μL 0.01 M MES pH 5.0 buffer+discard.
Add 200 μL of the EDC/NHS solution to the appropriate wells.
Incubate for 15 mins at room-temperature with gentle shaking (160 rpm).

Methods. Aptamer Immobilization
Discard the EDC/NHS solution from the plate (do not wash the plate at this stage).
Add 200 μL of the aptamer solution to the appropriate wells and reserve 20 μL.
Incubate for 80 mins at room-temperature with gentle shaking (160 rpm).

Methods. Plate Quenching:
Discard the aptamer solutions (save 20 μL for quality control—see below).
Rinse the COOH-plate 3× with 200 μL 0.01M MES buffer+discard.
Add 200 μL of the blocking buffer (300 mM ethanolamine solution—pH 8.5) to the appropriate wells (a multi-channel pipette can be used for this step).
Incubate for 30 mins at room-temperature with gentle shaking (160 rpm).
Discard the blocking solution.
Rinse the COOH-plate 3× with 200 μL 10 mM Tris pH 8.5+discard.
Empty plates of wash by tapping upside down on a paper towel.
Seal plate and store at room temperature.

Methods. Quality Control:
Using one plate from each batch, do not add aptamer solution to well H12 of that plate. Otherwise, treat that well exactly the same as others (EDC/NHS activation, ethanolamine blocking, washing).
When the plate is finished, use wells G12 and H12 for quality control.
Rinse both wells with 300 uL 1×BWB and discard.
Add 200 μL fresh 1×BWB to both wells.
Add 1 μL 200 nM SR-DN dye to both wells, mix well using pipette.
200 nM SR-DN in 10% ethanol in water.
Read fluorescence absolute value and ratio between wells at 0, 5, 10, and 15 min intervals.
Read at 560 nm excitation/600 nm emission.
Read at 575 nm excitation/610 nm emission.

Methods. For DNA Immobilization Time Study:
Select a row from which to collect DNA solution.
Set aside 20 μL of DNA solution before adding onto plates.
Immediately after adding DNA solution to plates, take 20 μL from the first well and test the DNA concentration.
After 10 minutes, take 20 μL from the next well and test the concentration.
Repeat this for 80 min.

### Example 6: COVID-19/SARS-CoV-2 Quantum-Logic Aptamer Analyte Detection (QLAAD) Testing Kits

Examples 6-8 describe the detection of SARS-CoV-2 spike proteins by DNA aptamers using QLAAD Testing Kits. The QLAAD SARS-CoV-2 testing kits were specifically designed and engineered to work on standard laboratory equipment using readily available materials and fit within standard moderate and high-complexity laboratory workflows. In particular, the QLAAD assay of the QLAAD testing kit is a high throughput fluorescence-based antigen test designed for use with fluorescence microplate readers capable of fluorescence measurements. The QLAAD testing kit and QLAAD assay are intended for qualitative detection of SARS-CoV-2 spike protein antigen in anterior nares specimens stored in saline solution (e.g., 0.9% saline solution) from individuals who are suspected of having COVID-19. For symptomatic individuals, the specimens should be collected within the first seven (7) days of symptom onset for the most accurate results. The QLAAD assay may also be used for screening of individuals without symptoms or for other reasons to suspect COVID-19 infection. The SARS-CoV-2 QLAAD assay technology is based on affinity binding of the target, e.g., the SARS-CoV-2 spike protein measurand by a DNA aptamer probe.

The SARS-CoV-2 spike antigen is detectable in respiratory specimens during the acute phase of infection. Positive results show the presence of viral antigens, but clinical correlation with patient history and other diagnostic information is necessary to decide infection status. Positive results do not rule out bacterial infection or co-infection with other viruses. Negative results do not rule out SARS-CoV-2 infection and should not be used as the sole basis for treatment or patient management decisions, including infection control decisions. Negative results should be considered in the context of a patient's recent exposures, history, and the presence of clinical signs and symptoms consistent with COVID-19 and confirmed with a molecular assay, if necessary, for patient management.

Results obtained from the QLAAD testing kit and QLAAD assay are to identify SARS-CoV-2 spike antigen in the anterior nares specimen. The anterior nares specimen in saline solution is mixed with QLAAD sample buffer in wells of the microplate. The microplate has a probe pre-functionalized to the well. Methods of activating or otherwise preparing microplates and immobilizing the DNA aptamer probe are discussed above in Example 5. Within the microplate well, the DNA aptamer probe detects and captures the target SARS-CoV-2 antigen if present in the sample. Binding of the antigen by the DNA aptamer promotes access to a second binding domain for a reporter molecule. In the testing kits described herein, the reporter molecule is SR-DN. The reporter molecule is composed of a fluorophore attached to a quencher, which is activated upon binding to the probe. The solution is removed, disposed of, and replaced with detection reagent containing the reporter molecule, SR-DN, that is only activated by a DNA aptamer probe bound to the SARS-CoV-2 antigen.

The activation and binding event are stable and determined using a 96-well microplate reader with fluorescence capabilities. The positive determination is made by analyzing the change in fluorescence when a probe and fluorophore are added to the sample. To determine that a significant change has occurred, a baseline fluorescence is generated using a negative control sample. The negative control is an antigen that the probe has been tested against and it has consistently been proven not to interact with. The fluorescence from this sample is shown as a negative result. The positive control is a recombinant version of the protein that consistently generates an increase in fluorescence. This control confirms that the test reagents are functional and capable of generating a positive result.

The SARS-CoV-2 QLAAD assay is intended for use by trained clinical laboratory personnel specifically instructed and trained in the use of 96-well microplate readers for in vitro diagnostic procedures. Internally validated commercial microplate readers such as the Synergy H1, Hybrid Multi-Mode Reader and the Infinite M1000 Multimode reader. The SARs-CoV-2 QLAAD test kit is to be used with microplate readers capable of fluorescence, endpoint measurement that can export data to Microsoft Excel for further interpretation and analysis. Two examples of the QLAAD assay as provided by two QLAAD Testing Kits are described below in Examples 7 and 8.

### Example 7: QLAAD Testing Kit 1

In a first example, a first QLAAD Testing Kit that works as discussed above in Example 6 was developed (“QLAAD Testing Kit 1”). The materials, recommendations, instructions, analysis and interpretation of results for QLAAD Testing Kit 1 are described below.

Included Materials. Materials included in the QLAAD Testing Kit 1 are shown below in Table 2

TABLE 2
Material Components and Specifications

| Component Name | Component Description | Specification |
|---|---|---|
| CoV-2 QLAAD | Ready-to-use 96-well micro- | 10 plates |
| plate with probe | plate containing covalently | |
| | attached probe for the | |
| | detection of CoV-2 antigen | |
| QLAAD Sample | Salt-based buffer | 2 bottles, 20 mL |
| Buffer | | each, 10x |
| | | concentration |
| COV-2 Detection | Ready-to-use solution | 10-2 ml vials, |
| Reagent | containing reporter | 0.5 mL each |
| Excel analysis | Template containing analysis | 1 Excel template |
| template | algorithm for making | |
| | determinations | |

Materials Required but Not Included. The following materials are required to perform the QLAAD assay, but are not included in the QLAAD Testing Kit 1:
Precision Multichannel pipettes for volumes 5, 10, and 200 microliter volumes.
Disposable pipette tips suitable for the above volumes.
Deionized or distilled water (Molecular grade, RNase-free).
Microplate reader capable of fluorescence measurements (excitation 575 nm, emission 610 nm).
Positive control Recombinant protein.
Negative control.

Recommendations. The QLAAD Testing Kit 1 comes with the following recommendations:
Use of this kit should be carried out in a sterile and clean environment.
PPE such as gloves, masks, and goggles should be used to prevent contamination.
Materials found in this kit should be treated as if they were infectious or harmful chemicals.
Use Good Laboratory Practices while carrying out this procedure.

Instructions. Specimen Collection. Specimens should be collected as follows:
Anterior nares swabs must be collected by a trained and qualified professional.
Sample swabs must be stored in Saline solution. (0.9% NaCl).

Instructions. Sample Storage and Stability. Specimen samples should be stored as follows:
Specimens should be tested immediately following collections.
If necessary, specimens may be stored at 4° C. for up to 2 days.
Freeze thawing is not recommended.

Instructions. Validated Controls. The following positive controls have been validated for use with the SARS CoV-2 QLAAD test:
SARS-CoV-2 spike glycoprotein (S1), The Native Antigen Company, Sku:REC31806.
Gamma-irradiated SARS-CoV-2 (BEI Resources USA-WA1/2020 NRC-52287).
Additional positive controls are under review such as additional recombinant proteins and organisms from other manufacturers.

The following negative controls have been validated for use with the SARS CoV-2 QLAAD test:
SARS-CoV-2 spike glycoprotein (S2), The Native Antigen Company, Sku:REC1830.
Saline solution blank control.
Inactivated microbial organisms under review.

Instructions. Preparation. Preparation is as follows:
Prepare 1×QLAAD sample buffer by mixing 20 mL of 10×QLAAD sample buffer with 180 mL of molecular grade water.
Store at room temperature.

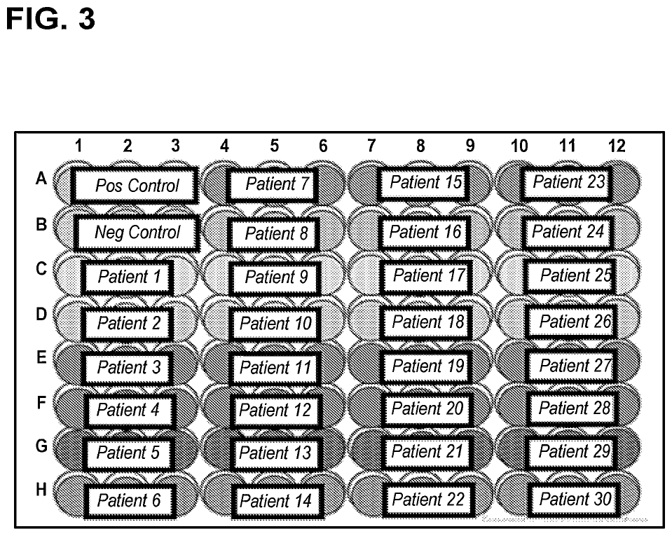

Instructions. User Instructions. Instructions for performing the assay are as follows:
Rinse each well of the plate with 200 μL of 1×QLAAD sample buffer.
Add 200 μL 1×QLAAD sample buffer using a multichannel pipette.
Mix by pipetting up and down.
Completely remove all liquid by pipetting.
Add 200 μL of 1×QLAAD sample buffer to each test and control well.
See FIG. 3 for recommended layout.
Add 10 μL of each control to the appropriate well in triplicate, mix well by pipetting.
Add 10 μL of each specimen to the appropriate test well in triplicate, mix well by pipetting.
Cover the plate and incubate at room temperature (20-25° C.) for 5 minutes.
Remove the supernatant from each well and discard in accordance with state and federal procedures.
Add 200 μL of QLAAD sample buffer to each test well.
Add 5 μL of Detection reagent to all wells and mix well by pipetting.
Place in the microplate reader 575±10 nm Excitation and 610±10 nm Emission.
Press start on the microplate reader and collect data.
Export data to the Excel template.
Discard of all materials in accordance with state and federal procedures.

FIG. 3 illustrates an example map of a 96-well plate in accordance with embodiments of the present technology and with Example 6. “Pos control” refers to the positive controls described in Example 6 and “neg control” refers to the negative controls described in Example 6.

Analysis and Interpretation of Results

All test controls should be examined prior to interpretation of patient results. If the controls are not valid, the patient results cannot be interpreted. In the condition that the positive control fails, the SARS-CoV-2 QLAAD assay should be repeated. In the event that a second failure occurs, internal laboratory procedures and positive controls should be evaluated.

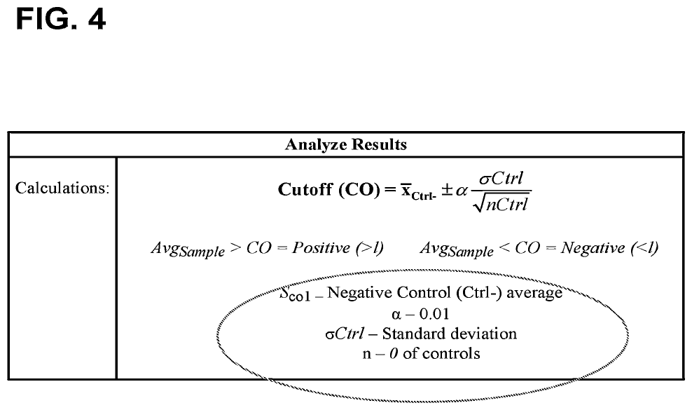

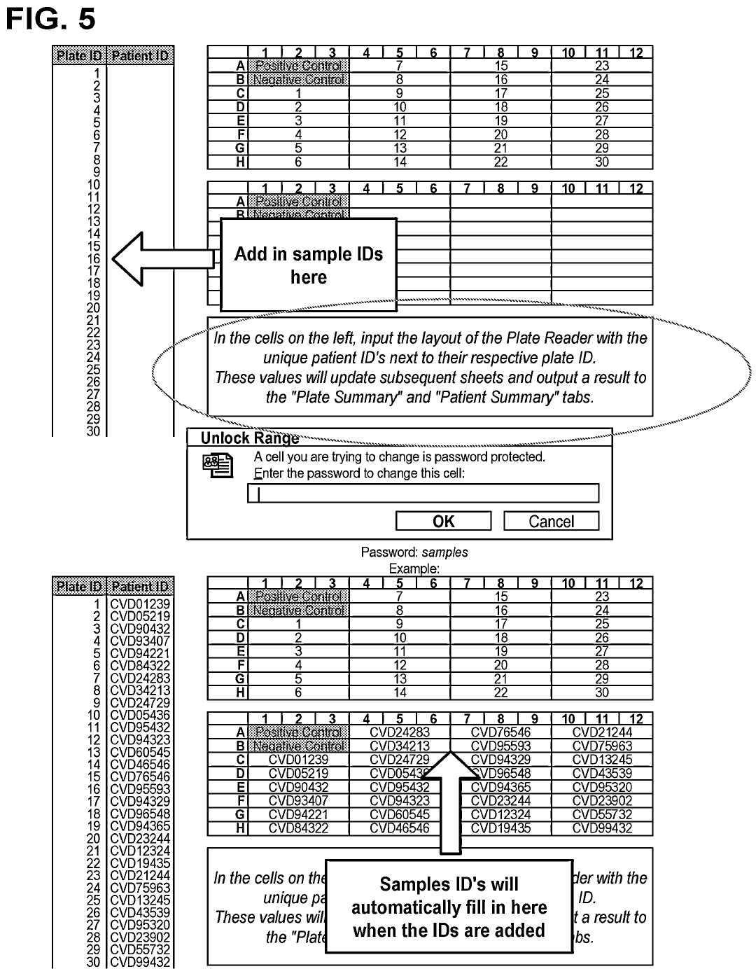

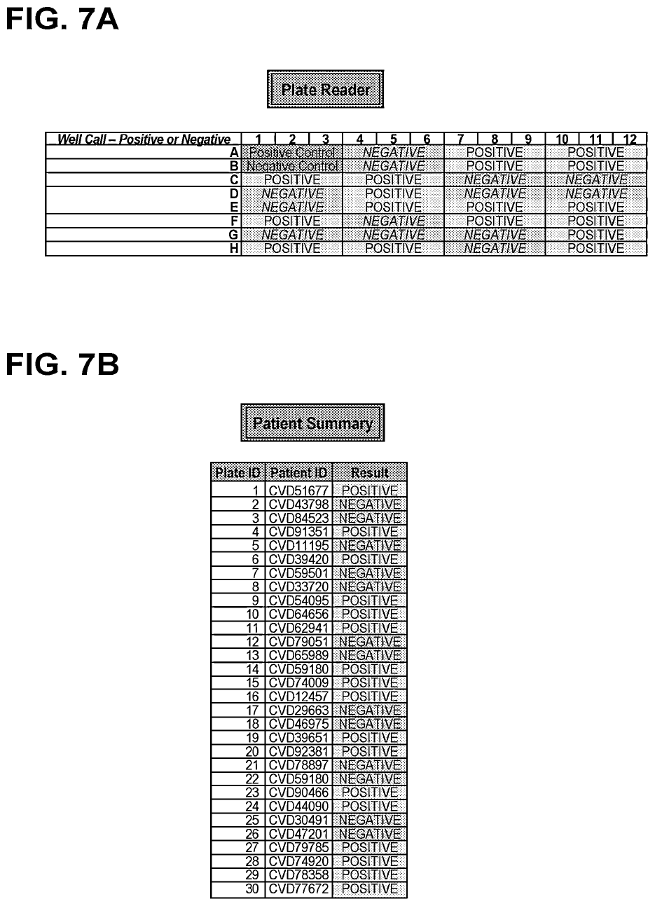

The SARS-CoV-2 QLAAD assay uses a validated macro-driven Excel spreadsheet to aid lab personnel to interpret the test results. Algorithms and screens shots from the validated macro-driven Excel spreadsheet are illustrated in FIGS. 4-10. For example, as shown in FIG. 5, the analysis sheet of the Excel spreadsheet can be opened and the user can proceed as follows:
Input the sample IDs into the table on the left side of the table “Plate Layout” next to their respective IDs under the column Patient ID. The sheet will ask for a password, input password samples to continue. Refer to the plate layouts on the top right side to figure out which wells belong to which plate ID.

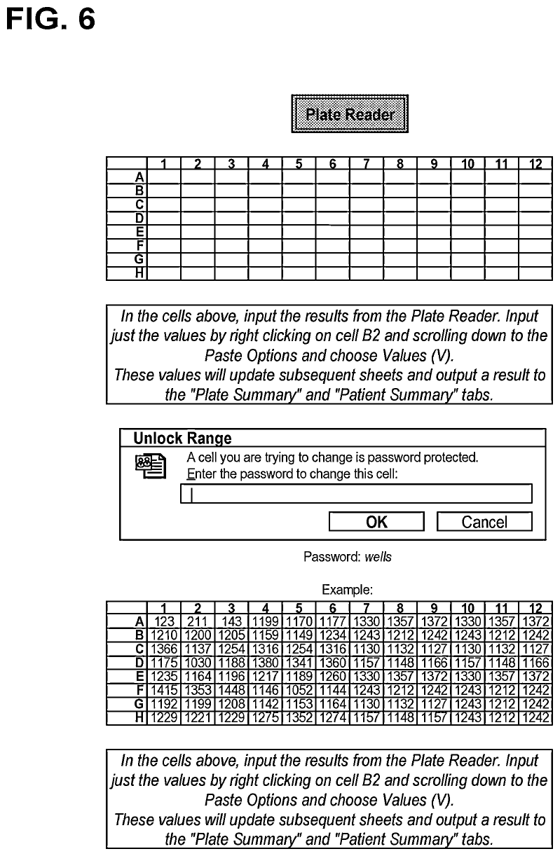

As shown in FIG. 6, the user can proceed as follows:
Input the output from the plate reader into the table on the page “Plate Reader.” The sheet will ask for a password, input password wells to continue. The output from the plate reader should be identical so copy the 96 values into the table.

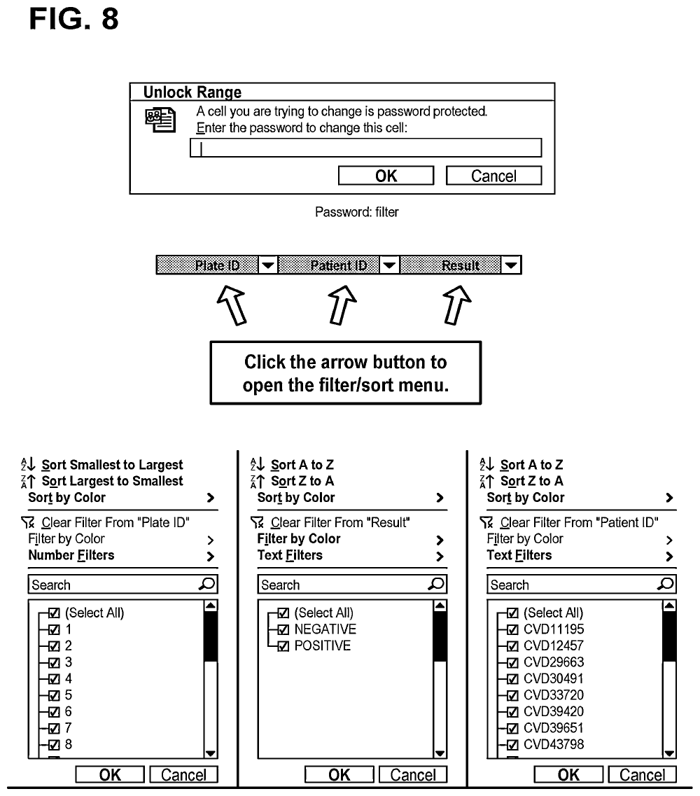

As shown in FIG. 8, the user can proceed as follows:
“Patient Summary” can be filtered by either Plate ID, Patient ID, or Result. This allows you to include/exclude certain Plate IDs or Patient IDs or to include only Positive or Negative Results.
“Patient Summary” can be sorted in either Plate ID, Patient ID, or Result to get a list that is in alphabetical order.
The form will ask for a password to edit the filter/sorts, input password filter to continue.

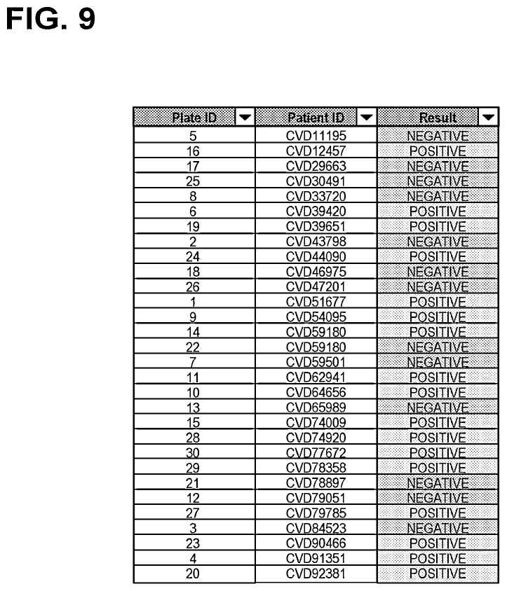

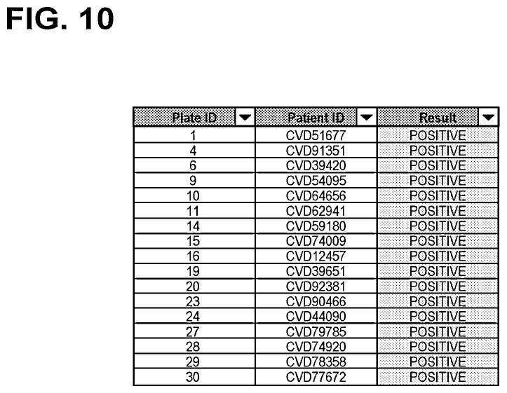

As shown in FIG. 9, the patient ID is sorted by alphabetical order and as shown in FIG. 10, the results are filtered by positive or negative.

### Example 8: QLAAD Testing Kit 2

In a second example, a second QLAAD Testing Kit that works as discussed above in Example 6 was developed (“QLAAD Testing Kit 2”). The materials, recommendations, instructions, analysis and interpretation of results and other validation tests performed using QLAAD Testing Kit 2 are described below.

Included Materials. Materials included in the QLAAD Testing Kit 2 are shown below in Table 3.

TABLE 3
Material Components and Specifications

| Component | Description | Quantity |
|---|---|---|
| Q-LAAD CoV-2 Probe | Functionalized microplate containing | (5) Individual plates, foil |
| Plate | bound DNA aptamer for the | packaged with |
| | detection of SARS-CoV-2 | desiccant. |
| Q-LAAD CoV-2 Buffer | pH 7.5, Sterile, salt-based buffer. | (1) bottle of 250 ml |
| | | total volume. |
| Q-LAAD COV-2 | 5 μM reporter molecule in solution of | (1) 2 mL bottle of 500 |
| Detection Reagent | 50% ethanol. | μL total volume |
| Microplate Seals | Acrylic, Adhesive, Clear seal to | (1) Package containing |
| | prevent microplate spillage. | 5 Microplate sealers |
| Package insert | Instructions for use | (1) paper pamphlet |
| Positive Quality Control | Recombinant SARS-CoV-2 spike-1 | (1) 2 mL vial |
| solution | protein (0.85 μg/mL) in saline. | |
| | Native Antigen Company, Catalog | |
| | No. REC31806. | |
| Negative Quality | Heat-inactivated Vero cells at a | (1) 2 mL vial |
| Control Solution | concentration of 2.4 × 105 cells/mL in | |
| | a saline. Vero, ATCC, Catalog No. | |
| | CCL-81. | |

The DNA aptamer included with the QLAAD CoV-2 Probe Plate includes the following sequence:

(SEQ ID NO: 42)
CACGGTTCGAGCTGGTACTAAACTTCGCTAAACAACACGCAGCATTTCA
TCGGGTCCAAAAGGGGCTGCTCGGGATTGCGGATATGGACCCGTCAGCT
TA,

and is referred to in Table 1 as N6-D2. The aptamer is also functionalized with a biotin to bind streptavidin-coated plates (i.e., N6-D2-biotin, discussed above) or an NH2 group (i.e., N6-D2-NH2, discussed above) to bind COOH-coated plates. It is noted that while the QLAAD Testing Kit 2 includes the N6-D2 aptamer, other kits may be generated that includes any of the DNA aptamers described herein (see, e.g., Table 1, Examples 1 and 2), on their own or in combination with one or more other DNA aptamers, i.e., a mixture of two or more of the aptamers may be used.

The DNA aptamer is a Fluorogenic Logic-gated Aptamer (FLA) with two distinct binding pockets that are made allosteric through a communication linker. This dual-logic aptamer was developed to allow for an easy and accurate detection of the binding of the target SARS-CoV-2 spike protein. The specific binding of SARS-CoV-2 spike protein triggers the folding of the second “logic” binding pocket to enable binding of a reporter molecule (or fluorophore). In this example, the reporter is SR-DN.

The FLA is biotin modified, and is attached to the plastic surface in the bottom of each well of standard 96-well microplates using biotin-streptavidin chemistry. The biotin-streptavidin arrives to the customer already conjugated and ready for use. No conjugations steps are needed by the technologist to perform this assay.

This test does not have a sample extraction, or preparation steps that require specific instruments or reagents to incubate the sample for analyte detection, reducing test complexity. The sample is simply added directly to the wells containing buffer and incubated for 10 minutes at room temperature. After specimen incubation and removal of unbound sample, the detection reagent is added. The detection reagent contains a fluorogenic small molecule which is subsequently recognized and bound by the second logic pocket. The second binding of the fluorophore is conditional upon binding of the SARS-CoV-2 spike protein. Once the fluorophore is bound, a change in fluorescent signal is measured using a microplate reader at the endpoint of the 5-minute room temperature incubation.

The high-throughput Q-LAAD SARS-CoV-2 test described herein combines the speed and relative simplicity of an antigen test with the sensitivity of a molecular test. This fluorescence-based assay utilizes a 96-well plate format that will fit into the normal workflow of any moderate-complexity lab. The test is easy to setup, it is significantly faster for the technician to prepare, and requires only 5 minutes on the instrument. The BioTek plate reader with Gen5 software provides results without technician interpretation. With an optimized workflow, the Q-LAAD SARS-CoV-2 assay can yield a throughput of 348 tests per hour per instrument.

Materials Required but Not Included. The additional materials shown below in Table 4 are required to perform the QLAAD assay, but are not included in the QLAAD Testing Kit 2. The source and catalog numbers are meant to be exemplary. Equivalent or substantially equivalent materials from other sources may be substituted.

TABLE 4
Additional Materials

| Component | Description | Source | Catalog# |
|---|---|---|---|
| Polystyrene Pipette | Argos Polystyrene, 55 mL capacity, | Fisher | 03-391-536 |
| Basin | Sterile, White, DNase-RNase free, | Scientific | |
| | Non-pyrogenic, 5 per pack, Used for | | |
| | Q-LAAD Buffer or similar | | |
| Polypropylene | Integra Polypropylene, 25 mL, 200 | Fisher | NC1529985 |
| Pipette Basin | per pack, Used for Q-LAAD Detection | Scientific | |
| | Reagent or similar | | |
| Saline Collection Vial | 0.9% Saline solution, 3 mL in 10 mL | Fisher | NC1909168 |
| | Tube, 50/pack or similar | Scientific | |
| Collection Swab | Flocked, Sterile, Nylon Fiber, | Fisher | 22-349-820 |
| | individually wrapped or similar | Scientific | |
| 5 mL Polypropylene | Argos Black 5 mL polypropylene snap | Fisher | 03-391-275 |
| Snap cap tube | cap tube or similar | Scientific | |
| Ethanol | 70% purity, ethanol solution, | Fisher | BP82014 |
| | Molecular Biology grade, CAS#64-17- | Scientific | |
| | 5 or similar | | |
| Pipette Tips, | ep Dualfilter T.I.P.S. ® LoRetention ®, | Fisher | 05-413-959 |
| 0.1-10 μL | PCR clean and sterile, 0.1-10 μL S, | Scientific | |
| | 34 mm, dark gray, colorless tips, 960 | | |
| | tips (10 racks × 96 tips) or similar | | |
| Pipette Tips, | ep Dualfilter T.I.P.S. ® LoRetention ® | Fisher | 05-413-952 |
| 2-200 μL | PCR clean and sterile, 2-200 μL, 55 | Scientific | |
| | mm, yellow, colorless tips, 960 tips | | |
| | (10 racks × 96 tips) or similar | | |
| Pipette Tips, | ep Dualfilter T.I.P.S. ® LoRetention ® | Fisher | 05-413-964 |
| 50-1000 μL | PCR clean and sterile, 50-1,000 μL, | Scientific | |
| | 76 mm, blue, colorless tips, 960 tips | | |
| | (10 racks × 96 tips) or similar | | |
| Multichannel Pipette, | Gilson, PIPETMAN L Multichannel, | Fisher | FA10014G |
| 0.5-10 μL | P12 × 10 L, 12 channel, 0.5-10 μL or | Scientific | |
| | similar | | |
| Multichannel Pipette, | Eppendorf Research ® plus, 8- | Fisher | 13-690-048 |
| 10-100 μL | channel, variable, incl. | Scientific | |
| | epT.I.P.S. ® Box, 10-100 μL, yellow | | |
| | or similar | | |
| Multichannel Pipette, | Gilson, PIPETMAN L Multichannel, | Fisher | FA10016G |
| 20-300 μL | P12 × 300 L, 12 channel, 20-30 0 μL or | Scientific | |
| | similar | | |

In addition, the QLAAD Testing Kit 2 is used with the components in Table 5 below in accordance with this working example and results, but substantially similar or equivalent components that function in the same manner as the descriptions below may also be used with similar efficacy.

TABLE 5
Components Used

| Component | Description | Source | Catalog# |
|---|---|---|---|
| BioTek Lx | Carries out readings in absorbance, | BioTek | SLXFA |
| Multi-mode | fluorescence, and luminescence | | |
| Microplate | | | |
| Reader | | | |
| Microplate | Custom Filter Cube: Excitation | BioTek | 1505000 |
| Cube Filter | wavelength 575 ± 10 nm; | | |
| | Dichroic mirror 595 nm, | | |
| | Emission wavelength 610 ± 10 nm | | |
| Microplate | Gen5 Software Features for | BioTek | N/A |
| Software | Detection; microplate reading | | |
| | and data analysis | | |

Instructions: Sample Storage and Stability. Specimen samples are collected using polypropylene plastic tubes containing 2-3 mL 0.9% saline and 6 inch plastic handle nylon flocked swabs based on the following instructions:
1) Remove swab from packaging.
2) Insert the swab into the anterior nares (high into the nose) cavity and rotate the swab for 15 seconds. Repeat on other anterior nares.
3) Break the swab on the indicated breakpoint.
4) Place the swab into the collection tube flocked side down. Store vertically.
5) Allow the swab to incubate in the collection buffer for 15-30 minutes at room temperature. Vortex sample with swab in the conical for 5 seconds.
6) Remove the swab from collection buffer using forceps in a sterile manner. If removing multiple swabs, sanitize forceps with 70% ethanol between each swab to prevent cross-contamination.

Samples should be tested as soon as possible. Sample integrity begins to decline after 90 minutes at room temperature. If testing within 90 minutes is not possible remove swab and store at 4 degrees C. for up to 24 hours. If samples will not be used within 24 hours remove swab and freeze each collection tube. Sample stability studies that have been conducted indicate that there is no significant difference between positive samples that are fresh, once frozen and thawed and twice frozen and thawed sample results.

Instructions. Control Materials. An external positive control is used to qualify the assay positive result is within the expected positive signal range of fluorescence values and is used each time the test is performed. The positive control is a solution of recombinant SARS-CoV-2 spike-1 protein (0.85 μg/mL) in saline. The SARS-CoV-2 spike-1 protein may be obtained from any reputable source, e.g., Novel Coronavirus (SARS-CoV-2) Spike Glycoprotein (S1), Native Antigen Company, Catalog No. REC31806.

An external negative control is used to qualify that the assay negative result is within the expected range of fluorescence values and is used each time the assay is performed. The negative control contains heat-inactivated Vero cells (Vero, ATCC, Catalog No. CCL-81) at a concentration of 2.4×10⁵ cells/mL in a saline solution.

An Internal Reference is used to normalize the average fluorescent value of each specimen and controls ran on the assay. The Internal reference is the detection reagent without addition of sample.

Instructions: Sample Preparation. To prepare samples for use in the QLAAD assay in the QLAAD Testing Kit 2: Preparation is as follows:
Allow samples to come to room temperature.
Prepare samples for testing by adding 35 microliters of each specimen or control to individual 200 microliter tubes.
Record test sample-well location with patient ID on to the well layout template.

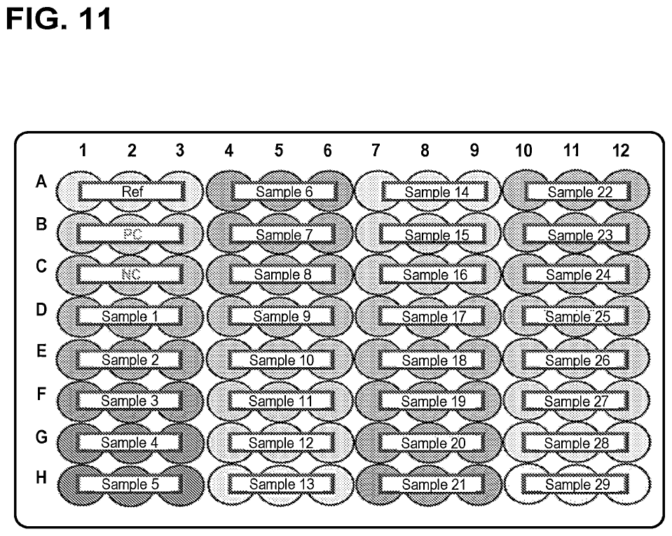

Instructions. Operator Instructions. When the samples are prepared, the QLAAD assay is performed as follows:
1. Review microplate sample layout in FIG. 11 below. The BioTek plate reader software must be programmed to match the layout shown in FIG. 11.
2. Open the plate package and remove plate from package. Discard packaging.
3. Pour Q-LAAD sample buffer to the polystyrene reservoir boat and then add 200 microliters of to each well of the dry microplate using a multichannel pipette to prepare the plate.
4. Remove all buffer from each well using a multichannel pipette.
5. Use a multichannel pipette to add 100 microliters of Q-LAAD sample buffer to all wells.
6. Use a multichannel pipette to add 10 microliters of each sample to the appropriate wells from the prepared tubes and mix up and down 3×.
7. Incubate the plate for 10 minutes at room temperature.
8. Following the 10-minute incubation, use a multichannel pipette to remove the 110 microliters of solution from each well and discard in accordance with local state and federal regulations.
9. Add 100 microliters of Q-LAAD sample buffer to each well using a multichannel pipette.
10. Prepare the detection reagent.
11. Shake the 25× detection reagent tube by hand for 3 seconds. Do not vortex.
12. Prepare a stock of 10% ethanol from the 96% ethanol. Add 1920 microliters of the 10% ethanol to a 5 mL snap-cap tube. Then add 80 microliters of 25× detection reagent to the 5 mL tube containing 1920 microliters of 10% ethanol and mix briefly by hand for 3 seconds. Do not centrifuge or vortex.
13. Add the diluted detection reagent to a polypropylene reservoir boat compatible with the multichannel pipette and use immediately.
14. Using a multichannel pipette, dispense 5 microliters of diluted detection reagent into each well and pipette up and down 3 times to mix. Avoid forming bubbles. Complete within 4 minutes. Any delay in this step beyond 4 minutes may yield invalid results.
15. Seal with clear plate sealer and incubate for 5 minutes in the dark at room temperature. Recommendation: incubate in the plate reader.
16. Place the microplate in the microplate reader (575±10 nm Excitation and 610±10 nm Emission), read the plate and collect the data and report.
17. Discard all hazardous waste materials in accordance with local state and federal regulations.

FIG. 11 illustrates an example map of a 96-well plate in accordance with embodiments of the present technology and with Example 6. “Pos control” refers to the positive controls described in Example 6 and “neg control” refers to the negative controls described in Example 6.

Controls and Internal Reference Results. All test controls will be examined prior to interpretation of patient results by the software. If the controls are not valid, the patient results cannot be interpreted, and test should be repeated.

The software will interpret the reference wells and control wells automatically. The value from the internal reference wells (REF) informs the algorithm and proceeds to produce positive, negative, or invalid results. The plate reader software will identify whether the REF mean is within the designated parameters which is comprised between or equal to 1,137 RFU and 5,221 RFU. If the REF mean is either less than 1,137 RFU or greater than 5,221 RFU the assay is invalid and the assay should be retested. If the positive control mean yields a value less than the Cutoff (1,995 RFU), the result is valid. If the positive control mean value is greater than 1,995 fluorescence units, the assay is invalid, and the samples should be retested. If the negative control mean yields a value greater than or equal to the Cutoff (1,995 RFU), the result is valid. If the negative control mean value is less than the Cutoff (1,995 RFU), the assay is invalid and the samples should be retested. If any of the averaged positive control wells, averaged negative control wells or averaged internal reference wells yields a value less than 350 RFU or greater than 6,000 RFU, the assay is invalid, and the samples should be retested. If any individual well yields a value of less than 100 RFU, the sample is invalid and the samples should be retested. This is summarized in the Table 6 below.

TABLE 6
Q-LAAD SARS-COV-2 Control Results Summary

| | | Control Result |
|---|---|---|
| | | Interpretation |
| Relative | | (Software will interpret |
| Fluorescence units | Well | control and internal |
| (RFU) | Description | reference wells automatically) |
| x < 1995 | Positive | Positive Control passed. |
| | Control | Plate is valid. |
| x ≥ 1995 | Positive | Positive Control failed. |
| | Control | Plate is invalid. |
| | | Retest. |
| x ≥ 1995 | Negative | Negative Control passes. |
| | Control | Plate is valid. |
| x < 1995 | Negative | Negative control failed. |
| | Control | Plate is invalid. |
| | | Retest. |
| 1137 ≤ x ≤ 5221 | Internal | Internal Reference passes. |
| | Reference | Plate is valid. |
| x < 1137 or 5221 < x | Average | Internal Reference failed. |
| | Internal | Plate is invalid |
| | Reference | Retest. |
| x < 350 or 6000 < x * | Average of | Test failure: Too little/too |
| | any Control | much detection reagent. |
| | Result | Retest. |
| <100* | Individual | Test failure: Too little/ |
| | well | or no detection reagent. |
| | | Retest. |

This “Invalid” result indicates insufficient volume of detection reagent, thereby minimizing the chances of false negatives due to insufficient reagent volume.

Result Interpretation. After the software processes the controls and internal reference wells and determines the plate is valid, the assay will continue to analyze the test samples wells. The software will interpret the test sample wells automatically and will output the final results. Samples with mean values less than the Cutoff (1,995 RFU) will be resulted as “Positive”. Samples with mean values equal to or greater than the Cutoff (1,995 RFU) will be resulted as “Negative”. If any of the averaged sample wells yields a value less than 350 RFU or greater than 6,000 RFU, the sample is invalid and the sample should be retested. If any individual well yields a value of less than 100 RFU, the sample is invalid and should be retested. Result interpretation is summarized in Table 7 below. Sample ID and with the result determination will be exported for use in Laboratory Information Systems.

TABLE 7
Q-LAAD SARS-COV-2 Results Interpretation Summary

| Relative Fluorescence | | |
|---|---|---|
| units (RFU) | Test Result | Action |
| 350 < x < 1995 | Positive | Positive Result for SARS- |
| | | COV-2. Accept Result. |
| 1995 ≤ x ≤ 6000 | Negative | Negative Result for SARS- |
| | | COV-2. Accept Result. |
| Average of wells* | Invalid | Test failure: Too little/too |
| x < 350 or 6000 < x | | much detection reagent. |
| Any individual well* | | Retest. |
| x < 100 | | |

This “Invalid” result indicates insufficient volume of detection reagent, thereby minimizing the chances of false negatives due to insufficient reagent volume.

Lot to Lot Analysis. Information on lot to lot reproducibility was compiled. Multiple lots (batches) were produced and tested during verification and validation. In addition, quality control procedures were in place for lot release.

The quality between the lots of probe plates were verified by assessing both the relative amount of DNA effectively bound in each well as well as the performance of the plate. Each of these measurements are analyzed and fall into a predetermined fluorescent unit range: The acceptance criteria are at least 85% of the wells within a plate must fall between 0.8 and 1.3 picomoles of DNA as measured by the GelGreen QC and the average of the N=12 Positive Controls, N=12 Negative Controls and N=72 Internal Reference follow the same rules are described in the Table 6 (Q-LAAD SARS-CoV-2 Control Results Summary). 10% (or 50 plates maximum) of each lot of plates are being utilized for QC.

The correct concentration of the Q-LAAD detection reagents is verified by absorbance measurement and compared to the previous lot. The acceptance criteria is that new lots must be within 15% of the established reference batch to pass.

The quality of buffer is assessed by pH verification. Lots must have a 7.6<pH<7.9 to pass.

Lot production of kits was reproducible and performance was consistent across multiple lots. No statistically significant differences were detected between several lots for all the major components.

Software Validation. The software powering the assay described in this example is produced by BioTek. The microplate reader and Gen5 software package (software version number 3.10.06) used is labeled as “in Vitro diagnostics use”. The assay protocol for use with Gen5 software was created and provided herein. The protocol is controlled and includes the system procedure and algorithm to generate test results. Q-LAAD SARS-CoV-2 test performance has been verified and validated using the instruments and software as specified in the IFU.

Inputs and outputs of the test protocol were verified for use with Q-LAAD SARS-CoV-2 test on the BioTek platform through testing of positive controls, negative controls, reference material and clinical samples. Clinical and end user validation testing has been performed using the entire product, multiple users and several lots of manufactured test kits.

New users will receive the protocol at the time of initial installation. Updates to the protocol will be made available through controlled release directly to existing users. Updated protocols will also be made available through a website for direct download.

Testing Cagabilities. 29 individual samples can be tested on a single Q-LAAD assay plate, each in triplicate with controls. Unlike a RT-qPCR based assay, the Q-LAAD test only requires 5 minutes on the instrument per assay. The throughput per instrument can be up to 348 tests per hour.

Reagent Stability (prophetic). Reagent Stability Testing will be performed using procedures that are cGMP 21CFR820, GLP and ISO 13485 compliant.

First, the stability of open kits will be tested. The goal of the Kit stability testing is to test all kit components together to determine the stability of the entire kit at various temperatures. The kits will be stored under defined temperatures including real-time/real-temperature (2-8° C.) and accelerated conditions (15-30° C.) and 37° C. The kits will be stored at the appropriate temperatures until testing is performed at predetermined time points.

In addition, the stability of opened reagents will be tested. The goal of the Open Reagent stability testing is to test kit components that are stored after being “opened” a) microplate removed from the foil pouch and b) caps removed from reagents and stored at the appropriate temperature. The Open Reagent stability will be stored under defined temperatures including real-time/real-temperature (2-8° C.) and accelerated conditions (15-30° C.). Testing protocols will be generated to test (i) QLAAD SARS-CoV-2 Components and their shelf life at various temperatures, (ii) QLAAD SARS-CoV-2 Detection Kit and its reagents after they have been opened, and (iii) QLAAD SARS-CoV-2 Detection Kit after it has gone through shipping.

Limit of Detection (LoD)—Analytical Sensitivity. Contrived heat-inactivated and gamma-irradiated virus samples showed high antigenic variability when detecting spike protein with the aptamer-based technology disclosed herein. Other antigen tests detecting SARS-CoV-2 detect the nucleoprotein, which is less susceptible to gamma-irradiation mediated protein degradation compared to spike proteins (references: doi:10.3389/fphy.2020.565861, doi.org/10.1107/S2059798316003351). Consequentially, naturally occurring clinical anterior nares specimens were used with live virus in order to determine LoD. Please refer to LoD protocol discussed above for details on the procedure.

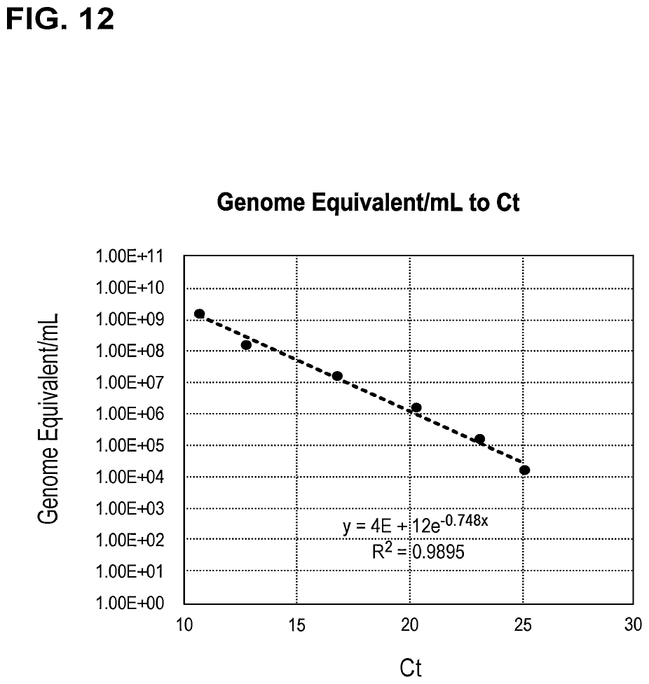

The Q-LAAD SARS-CoV-2 limit of detection (LoD) was determined by evaluating different concentrations of clinical specimens. First, a standard curve was generated by making 5, ten-fold serial dilutions of gamma-irradiated virus from BEL. The CDC recommended RT-PCR kit and procedure was used, which utilizes N1, N2, RNAP genes for target amplification and fit a curve to the data to give us the equation for the standard curve, as shown below in Table 7 and FIG. 12. Each analytical study was performed using real clinical samples. Each sample was tested with the CDC assay (the same assay that was used to generate the standard curve below) and viral load was quantified.

TABLE 7
Q-LAAD SARS-COV-2 Results Interpretation Summary

| Sample ID | Ct N1 | Ct N2 | Avg Ct | GE/mL |
|---|---|---|---|---|
| Negative Control | 55 | 55 | 55 | 0 |
| Stock | 10.3 | 11.2 | 10.8 | 1.57E+09 |
| Dilution 1 | 12.6 | 13.0 | 12.8 | 1.57E+08 |
| Dilution 2 | 16.4 | 17.2 | 16.8 | 1.57E+07 |
| Dilution 3 | 19.8 | 20.8 | 20.3 | 1.57E+06 |
| Dilution 4 | 22.6 | 23.7 | 23.2 | 1.57E+05 |
| Dilution 5 | 24.4 | 25.8 | 25.1 | 1.57E+04 |
| Positive Control | 20.8 | 21.3 | 21.1 | NA |

The range finding LoD was determined by testing three specimens that were serially diluted to viral loads ranging from 340 genome equivalents/mL to 0.207 genome equivalents/mL (GE/mL).

Confirmation of the limit of detection was tested with 20 replicates of clinical samples. Clinical samples with 36 GE/mL and 7.22 GE/mL were tested according to the test procedure to confirm the LoD.

The LoD was determined as the lowest virus concentration that was detected Z 95% of the time (i.e., concentration at which at least 19 out of 20 replicates tested positive).

The LoD for the Q-LAAD SARS-CoV-2 test in anterior nares specimens stored in 0.9% saline solution was confirmed as 36 GE/mL.

TABLE 8
LoD Range Finding

| Ct | Concentrations (GE/mL) | Agreement |
|---|---|---|
| 31.0 | 340.0 | 3/3 |
| 32.7 | 99.0 | 3/3 |
| 34.3 | 28.8 | 3/3 |
| 36.0 | 8.4 | 1/3 |
| 37.6 | 2.4 | 3/3 |
| 39.3 | 0.7 | 1/3 |
| 40.9 | 0.2 | 2/3 |

TABLE 9
LoD Confirmation

| Viral Concentration (GE/mL) | 36.1 | 7.2 |
|---|---|---|
| Positive Results | 20/20 | 16/20 |

The GE/mL do not match exactly between the LoD Range finding versus LoD Confirmation as the range finding was a dilution series of a clinical sample, whereas the LoD Confirmation was performed with a clinical sample with no dilution that was the closest match to the LoD determined by the range finding.

Cross-Reactivity and Interference by Microorganisms. A study was undertaken to determine if there is any cross-reactivity between high priority pathogens and other organisms when using the Q-LAAD SARS-CoV-2 test. Cross-reactivity (Analytical Specificity) is determined by using the Q-LAAD SARS-CoV-2 test in the presence of high priority pathogens likely in the circulating area. Pathogens from the same genetic family are included as well as common pathogens. In general, cross-reactivity was tested at worst-case scenario at 10⁵ (PFU/mL for virus) for viruses and 10⁶ (CFU/mL for bacteria). Microbes were spiked into known negative matrix for testing. To estimate the cross-reactivity of organisms not available for wet testing, in silico analysis using Basic Local Alignment Search Tool (BLAST) managed by the National Center for Biotechnology Information (NCBI) was used to assess the degree of protein sequence identity.

Microbial interference was tested by spiking pooled pathogens into positive SARS-CoV-2 anterior nares swab in saline (0.9%). Low viral-load positive samples were used at 1-3×LoD (GE 36/mL-GE 10⁸/mL). Pathogens were pooled at high interfering concentrations to represent worst-case scenario. Pooled organisms were tested with three replicates and the results are shown in Table 10 below.

TABLE 10

| | | | | Concentration | | |
|---|---|---|---|---|---|---|
| | | | | tested (CFU/ | | |
| Wet Testing | | | Catalog | mL and | Cross- | |
| Organism | Strain | Source | number | PFU/mL) | reactivity | Interference |
| Human | 229E | Isolate | ATCC Cat# | 10⁵ | 0/3 | 3/3 |
| coronavirus | | | VR-740 | | | |
| Human | OC43 | Isolate | ATCC Cat# | 10⁵ | 0/3 | 3/3 |
| coronavirus | | | VR-1558 | | | |
| Rhinovirus | B632 | Isolate | ATCC Cat# | 10⁵ | 0/3 | 3/3 |
| | | | VR-1645 | | | |
| Haemophilus | Rd [KW20] | Isolate | ATCC Cat# | 10⁵ | 0/3 | 3/3 |
| influenzae | | | 51907 | | | |
| Streptococcus | 262 [CIP | Isolate | ATCC Cat# | 10⁶ | 0/3 | 3/3 |
| pneumoniae | 104340] | | 49619 | | | |
| | (Klein) | | | | | |
| | Chester | | | | | |
| Streptococcus | Bruno [CIP | Isolate | ATCC Cat# | 10⁶ | 0/3 | 3/3 |
| pyogenes | 104226] | | 19615 | | | |
| Candida | CBS 562 | Isolate | ATCC Cat# | 10⁶ | 0/3 | 3/3 |
| albicans | [572, CCRC | | 18804 | | | |
| | 20512, | | | | | |
| | CECT 1002, | | | | | |
| | DBVPG | | | | | |
| | 6133, IFO | | | | | |
| | 1385, IGC | | | | | |
| | 3436, JCM | | | | | |
| | 1542, NCYC | | | | | |
| | 597, NRRL | | | | | |
| | Y-12983] | | | | | |
| Pooled human | N/A | N/A | N/A | N/A | 0/3 | 3/3 |
| nasal swab | | | | | | |
| Adenovirus 5 | Adenoid 75 | Isolate | ATCC Cat# | 10⁵ | 0/3 | 3/3 |
| | | | VR-1516 | | | |
| Parainfluenza | ATCC-2011- | Isolate | ATCC Cat# | 10⁵ | 0/3 | 3/3 |
| virus 3 | 5 | | VR-1782 | | | |
| Parainfluenza | C35 | Isolate | ATCC Cat# | 10⁵ | 0/3 | 3/3 |
| virus 1 | | | VR-94 | | | |
| Influenza A | A/WVS/33 | Isolate | ATCC Cat# | 10⁵ | 0/3 | 3/3 |
| (H1N1) | | | VR-1520 | | | |
| Influenza B | B/Florida/ | Isolate | ATCC Cat# | 10⁵ | 0/3 | 3/3 |
| | 78/2015 | | VR-1931 | | | |
| Enterovirus | H | Isolate | ATCC Cat# | 10⁵ | 0/3 | 3/3 |
| | | | VR-1432 | | | |
| Respiratory | Long | Isolate | ATCC Cat# | 10⁵ | 0/3 | 3/3 |
| syncytial virus | | | VR-26 | | | |
| Bordetella | 18323 | Isolate | ATCC Cat# | 10⁶ | 0/3 | 3/3 |
| pertussis | [NCTC | | 9797 | | | |
| | 10739] | | | | | |
| Mycoplasma | Eaton Agent | Isolate | ATCC Cat# | 10⁶ | 1/3 | 3/3 |
| pneumoniae | [NCTC | | 15531 | | | |
| | 10119] | | | | | |
| Staphylococous | NCTC 8532 | Isolate | ATCC Cat# | 10⁶ | 0/3 | 3/3 |
| aureus | [IAM 12544, | | 12600 | | | |
| | R. Hugh | | | | | |
| | 2605] | | | | | |
| Staphylococcus | FDA strain | Isolate | ATCC Cat# | 10⁶ | 0/3 | 3/3 |
| epidermidis | PCI 1200 | | CRM-12228 | | | |
| **Human | NL63 (heat- | Isolate | ZeptoMetrix | 1.70⁵ TOID | 0/3 | 3/3 |
| coronavirus | inactivated) | | Cat# | 50/mL | | |
| | | | 0810228CFHI | | | |
| Parainfluenza | Greer | Isolate | ATCC Cat# | 10⁵ | 0/3 | 3/3 |
| virus 2 | | | VR-92 | | | |
| Parainfluenza | 19503 | Isolate | BEI Cat# | 10⁵ | 0/3 | 3/3 |
| virus 4b | | | 3238 | | | |
| Influenza A | A/Hong | Isolate | ATCC Cat# | 10⁵ | 0/3 | 3/3 |
| H3N2 | Kong/8/68 | | VR-1679 | | | |
| **MERS-COV | EMC/2012 | Gamma- | BEI Cat# | 10⁵ | 0/3 | 3/3 |
| | (gamma- | irradiated | NR-50549 | | | |
| | irradiated) | | | | | |
| *SARS-COV | Urbani | Gamma- | BEI Cat# | 10⁵ | 0/3 | 3/3 |
| | (gamma- | irradiated | NR-9323 | | | |
| | irradiated) | | | | | |
| Human | TN/83-1211 | Isolate | BEI Cat# | 10⁵ | 0/3 | 3/3 |
| Metapneumovirus | | | NR-22227 | | | |
| Chlamydiaceae, | AR-39 | Isolate | ATCC Cat# | 10⁶ | 0/3 | 3/3 |
| Chlamydia | | | 53592 | | | |
| Legionella | Concord 3 | Isolate | ATCC Cat# | 10⁴ | 0/3 | 3/3 |
| pneumophila | [NCTC | | 35096 | | | |
| | 11985] | | | | | |

Cross-reactivity was observed for Mycoplasma pneumoniae at worst-case scenario concentration. To determine the concentration of Mycoplasma pneumoniae that does not cross-react, a dilution study was performed. The results of the serial dilution showed that no cross-reactivity was observed at concentrations less than 10⁵ CFU. No cross-reactivity was observed for all remaining microbes tested. No microbiological interference was observed with pooled microbes tested.
No cross-reactivity was detected for SARS-CoV, even though the in silico comparison between SARS-CoV-2 surface glycoprotein and SARS-CoV indicate a moderate level of homology and cross-reactivity may be likely.
No cross reactivity was detected for MERS or Human Coronavirus NL63, and in silica comparison show low sequence homology to SARS-CoV-2, which suggest that cross-reactivity is unlikely.

Endogenous Interference Substances Studies. Studies were performed to demonstrate that nineteen (19) potentially interfering substances do not cross-react or interfere with the detection of SARS-CoV-2 in the Q-LAAD SARS-CoV-2 assay at the stated concentrations. The results are shown in Table 11 below. Low viral-load positive samples were used at 1-3×LoD (GE 36/mL-GE 76/mL). No interfering substances were observed at the following concentrations.

TABLE 11
Test for Interfering Substances

| Potential | | Concen- | | |
|---|---|---|---|---|
| interfering | | tration | Negative | Positive |
| substances | Active Ingredient | tested | Result | Result |
| Afrin | Oxymetazoline | 5% v/v | 3/3 | 3/3 |
| Azithromycin | Azithromycin | 250 ug/mL | 3/3 | 3/3 |
| Blood | Blood | 0.005% | 3/3 | 3/3 |
| (Human)* | | | | |
| Chroaspetic | Menthol | 1.5 mg/mL | 3/3 | 3/3 |
| Clathromycin | Clathromycin | 1 mg/mL | 3/3 | 3/3 |
| Flonase | Fluticasone | 5% v/v | 3/3 | 3/3 |
| | Propionate | | | |
| Homeopathic | Alkalol | 1:10 dilution | 3/3 | 3/3 |
| Listerine | Menthol, Thymol, | 0.1% v/v | 3/3 | 3/3 |
| | methyl salicylate | | | |
| Mucin | Purified mucin | 2.5 mg/mL | 3/3 | 3/3 |
| | protein | | | |
| Mupirocin | Mupirocin | 1 mg/mL | 3/3 | 3/3 |
| Nasal Drops | Phenylephrine | 15% v/v | 3/3 | 3/3 |
| | hydrochloride | | | |
| Nasal Spray | Cromolyn | 15% v/v | 3/3 | 3/3 |
| Nelimed | Hyaluronic Acid | 5% v/v | 3/3 | 3/3 |
| Phenol Throat | Phenol | 5% v/v | 3/3 | 3/3 |
| Spray | | | | |
| Saline Nasal | Saline | 5% v/v | 3/3 | 3/3 |
| Spray | | | | |
| Tamiflu | Oseltamivir | 1 mg/mL | 3/3 | 3/3 |
| Tobramycin | Tobramycin | 4 mg/mL | 3/3 | 3/3 |
| Zicam | Oxymetazoline | 1% v/v | 3/3 | 3/3 |
| | hydrochloride | | | |
| Zicam All | Hydroxpropryl | 0.5% v/v | 3/3 | 3/3 |
| clear | methylcellulose, | | | |
| | disodium | | | |
| | phosphate | | | |

*Blood is a well-established inhibitor of fluorescence. Hemoglobin has been shown to be a potent quencher of free dyes such as EvaGreen, rhodamine based dye, and ROX (doi: 10.1007/s00216-018-0931-z). Q-LAAD technology utilizes free rhodamine dye molecules in the mechanism for signal detection. The inhibition of signal due to hemoglobin will register as a cross-reactive substance and produce a false positive. To mitigate false positives, samples suspected of having blood present should be recollected or tested using a different method. False negatives of signal due to the presence of blood are not reported up to 4% v/v.

High-dose Hook Effect. The potential for a high-dose hook effect was evaluated using clinical samples with high viral load as determined by RT-qPCR. The highest obtainable concentrations were evaluated. A range of 1.13×10⁸-1.20×10⁷ genome copies/mL was tested for High-dose Hook Effect.

A High-dose Hook Effect was not observed at the highest tested concentration of 1.13×10⁸ genome copies/mL.

Specimen Stability. A stability study was performed to determine the time, temperature and freeze/thaw dependent stability of anterior nares specimens stored in saline solution for use with the Q-LAAD SARS-CoV-2 test. It was concluded that the sample can be run at room temperature in less than 90 min. Specimen should be kept at 4° C. for up to 24 h; for longer durations samples must be frozen and stored at −20° C. or lower.

Clinical Evaluation: The Q-LAAD SARS-CoV-2 test was used to evaluate clinical samples. In a first trial, the clinical performance of the Q-LAAD SARS-CoV-2 test was established with a total of 70 anterior nasal swab samples. Anterior nares nasal swabs were collected and eluted in saline buffer (0.9%) and stored until tested. Samples were prepared and tested with Q-LAAD SARS-CoV-2 test according to the operator instructions. All samples were confirmed as positive (≤7 days from onset of symptoms), or negative by an RT-qPCR comparator (e.g., Lyra, Alinity, M2000 and/or DiaSorin) method for the study. Comparator testing on the samples was performed using QLAAD and a portion of the samples were tested using sensitive RT-qPCR comparator testing to ensure freeze thaw cycles did not degrade the sample. All testing was performed by operators blinded to the comparator RT-qPCR test results.

TABLE 12
Evaluation of Accuracy
Positive samples in data set by days onset symptoms

| Days symptom | Not | | | % |
|---|---|---|---|---|
| onset | Detected | Detected | Total | Correct |
| 1-3 | 4 | 18 | 22 | 82% |
| 4-5 | 0 | 6 | 6 | 100% |
| 6-7 | 0 | 2 | 2 | 100% |

TABLE 13
Evaluation of Sensitivity and
Specificity

| | Comparator RT-qPCR | |
|---|---|---|
| QLAAD | + | − |
| + | 26 | 2 |
| − | 4 | 38 |
| | Sensitivity | Specificity |
| | 87% | 95% |

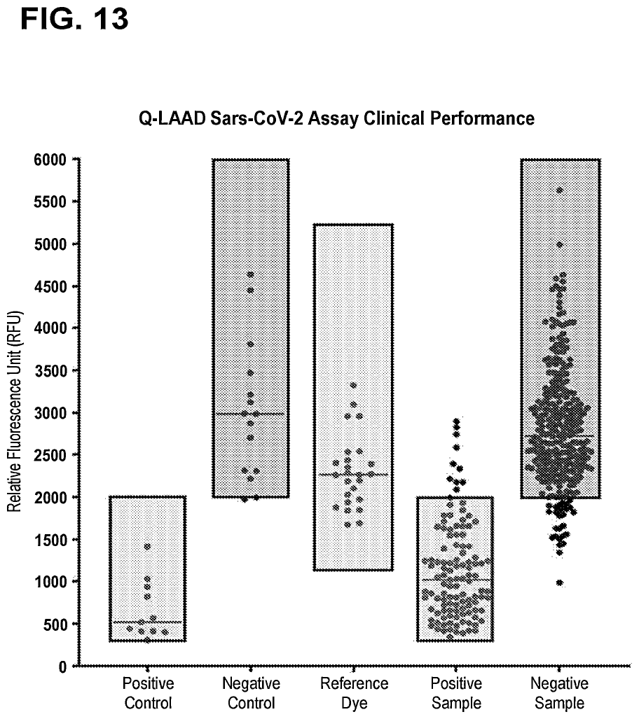

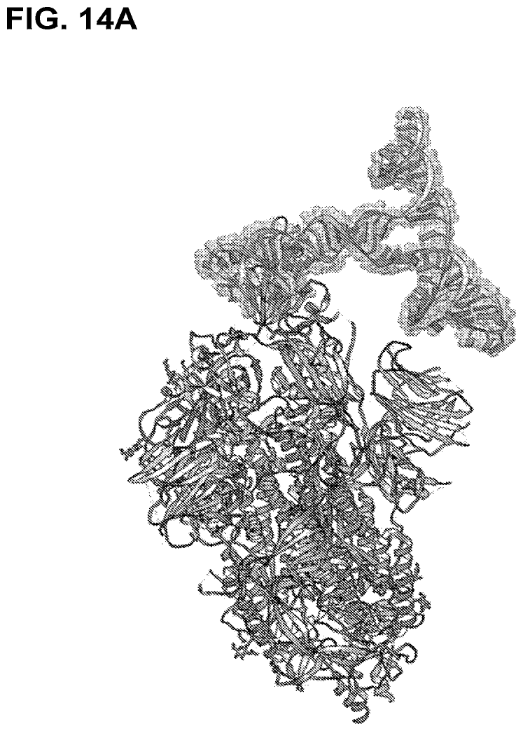

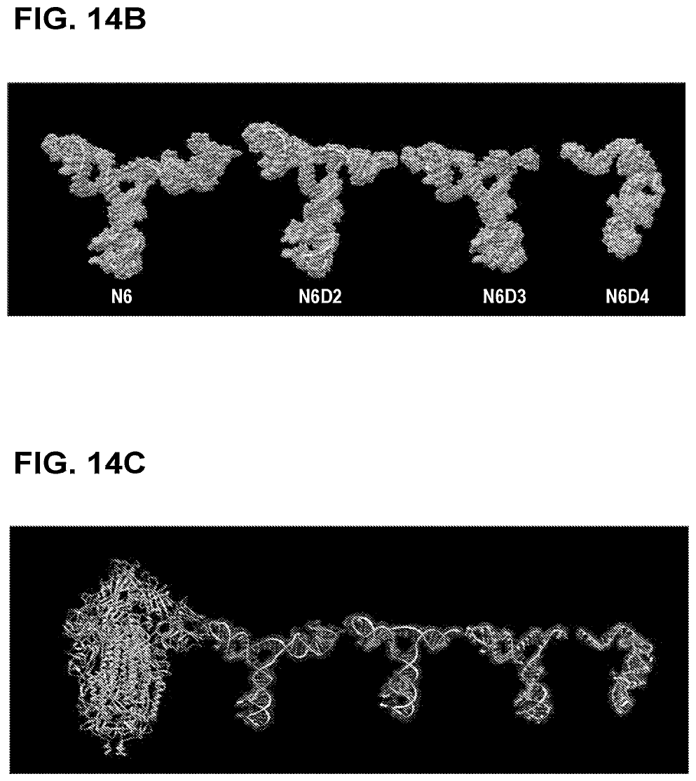

In a second trial, the clinical performance of the Q-LAAD SARS-CoV-2 test was established with a total of 411 anterior nasal swab samples collected and prepared for testing as discussed above. The results indicate greater than 90% accuracy for diagnosing positive and/or negative cases, as shown in FIG. 13 and Table 14.

TABLE 13
Evaluation of Accuracy

| True Negative | False Positive | Sum |
|---|---|---|
| 269 | 27 | 296 |
| 90.88% | 9.12% | |
| True Positive | False Negative | Sum |
| 105 | 10 | 115 |
| 91.30% | 8.70% | |

Variants Statement. Genetic variants of SARS-CoV-2 have been emerging and circulating around the world throughout the COVID-19 pandemic. Viral mutations and variants in the United States are routinely monitored through sequence-based surveillance, laboratory studies, and epidemiological investigations. A variant has one or more mutations that differentiate it from other variants in circulation. As expected, multiple Variants of Concern (VOC) for SARS-CoV-2 have been documented in the United States, and globally throughout this pandemic.

Clinical validation was realized using samples recently collected from the U.S. may indicate that the tests described herein already detect some variant strains. Moreover, because the Q-LAAD SARS-CoV-2 test is an antigenic test that was developed to bind to the receptor binding domain of the spike protein—more specifically, to amino acids 319 to 541—and it is not expected to that a significant change to binding of the aptamer would result if mutations occur outside of this amino acid region.

1. A DNA aptamer having a nucleotide sequence comprising:
at least a portion of a SARS-CoV-2-RBD aptamer backbone; and
a randomized region;
wherein the aptamer is capable of binding sulforhodamine-dinitroaniline (SR-DN) and a receptor binding domain (RBD) of a SARS-CoV-2 spike protein.

2. The DNA aptamer of claim 1, further comprising a forward primer handle at the 5′ end, a reverse primer handle at the 3′ end, or both a 5′ and a 3′ primer handle.

3. The DNA aptamer of claim 2, wherein the 5′ primer handle comprises SEQ ID NO:21, SEQ ID NO:88, and/or SEQ ID NO:22.

4. The DNA aptamer of any one of claim 2, wherein the SARS-CoV-2-RBD aptamer backbone is a SARS-CoV-2-RBD-1C (1C) backbone or a SARS-CoV-2-RBD-4C (4C) backbone.

5. The DNA aptamer of claim 4, wherein the 1C or 4C aptamer backbone is modified (a) to remove at least 5 nucleotides from the 3′ and/or 5′ ends or (b) to insert, delete, or substitute 1 or more nucleotides.

6. The DNA aptamer of claim 5, wherein the aptamer backbone comprises SEQ ID NO:1, SEQ ID NO:2, SEQ ID NO:3, or SEQ ID NO:4.

7. (canceled)

8. The DNA aptamer of claim 1, wherein the randomized region comprises (a) any one or more of SEQ ID NOs: 7-17; or (b) 38 nucleotides, 34 nucleotides, or 33 nucleotides.

9. (canceled)

10. The DNA aptamer of claim 1, wherein the randomized region is divided into a first randomized region and a second randomized region.

11. The DNA aptamer of claim 10, wherein (a) the first randomized region comprises 21 nucleotides, 19 nucleotides, or 16 nucleotides; or (b) the second randomized region comprises 17 nucleotides or 13 nucleotides, or (c) both (a) and (b).

12. (canceled)

13. The DNA aptamer of claim 1 wherein the DNA aptamer comprises a nucleotide sequence of

| (SEQ ID NO: 3) | |
|---|---|
| | NY₁CCGACCTTGTGCTTTGGGAGTGCTGGTCCAAGGGCGTTAANY₂ |
| | or |
| (SEQ ID NO: 4) | |
| | NY₃ACGCAGCATTTCATCGGGTCCAAAAGGGGCTGCTCGGGATTGC |
| | GGATATGGNY₂. |

14. The DNA aptamer of claim 1, wherein the nucleotide sequence comprises
(a) any one of SEQ ID NOs:20, 23-27;
(b) SEQ ID NOs:28-34;
(c) the nucleotide sequence in Table 1;
(d) SEQ ID NOs: 35-42, 89-90;
(e) a functional group that facilitates attachment to a plate, selected from an NH2 group or a biotin; or
(f) any one of SEQ ID NOs:91-100.

15.-24. (canceled)

25. A biosensor, comprising:
a reporter; and
a DNA aptamer with at least one stem, the aptamer having—
a target domain comprising a randomized region of at least 15 nucleotides disposed within the at least one stem,
a reporter domain configured to bind to the reporter, and
a linker domain between the target domain and the reporter domain.

26. The biosensor of claim 25, wherein the DNA aptamer comprises a nucleotide sequence of any of the nucleotide sequences provided in Table 1.

27. The biosensor of claim 25, wherein the DNA aptamer comprises a nucleotide sequence of

| (SEQ ID NO: 3) | |
|---|---|
| | NY₁CCGACCTTGTGCTTTGGGAGTGCTGGTCCAAGGGCGTTAANY₂ |
| | or |
| (SEQ ID NO: 4) | |
| | NY₃ACGCAGCATTTCATCGGGTCCAAAAGGGGCTGCTCGGGATTGC |
| | GGATATGGNY₂, |

wherein each of NY₁, NY₂, and NY₃ represents at least a portion of the randomized region.

28-31. (canceled)

32. The biosensor of claim 25, wherein the DNA aptamer comprises a nucleotide sequence of
(SEQ ID NO: 33) [symbol]
NNNNNNNNNNNNNNNNNNNNNNNNNNNNNNN
NNNNNNNNNNNNNNNNNNNNNNNNNNNNNGCCGTGCAGGTCCGA.

33. The biosensor of claim 27, wherein (a) NY₁ is 16 nucleotides and NY₂ is 17 nucleotides, or (b) NY₃ is 21 nucleotides and NY₂ is 17 nucleotides.

34. (canceled)

35. The biosensor of claim 25, wherein the randomized region is inserted on (a) an outside stem of the at least one stem of the DNA aptamer; (b) on an inside stem of the at least one stem of the aptamer.

36-37. (canceled)

38. The biosensor of claim 25, wherein the reporter is a fluorescent molecule.

39. The biosensor of any one of claim 25, wherein the linker is operably connected between the target domain and the reporter domain such that a conformational change occurs in the DNA aptamer in response to the target domain binding to a target, the reporter binding to the reporter domain, or a combination thereof.

40. A method of detecting a target in a sample comprising contacting the sample with the biosensor of claim 25, wherein the target is:
(a) a pathogen selected from a bacterial pathogen, a viral pathogen, a prokaryotic pathogen, and a fungal pathogen;
(b) a protein encoded by a SARS-CoV2 pathogen;
(c) a small molecule selected from a toxin, a pharmaceutical agent, a cannabinoid, bisphenol A, fluoride, or benzene;
(d) a solvent selected from acetone, cyclohexane, acetic acid, ethanol, or benzene; or
(e) an ion selected from potassium, chloride, sodium, lithium, magnesium, mercury, or lead.

41-50. (canceled)


SEQUENCE LISTING (105 sequences)

SEQ ID NO: 1 - DNA, 51 bp, Artificial Sequence
  Synthetic
cagcaccgaccttgtgctttgggagtgctggtccaagggcgttaatggaca

SEQ ID NO: 2 - DNA, 67 bp, Artificial Sequence
  Synthetic
atccagagtgacgcagcatttcatcgggtccaaaaggggctgctcgggattgcggatatggacacgt

SEQ ID NO: 3 - DNA, 42 bp, Artificial Sequence
  Synthetic
  misc_feature (1)..(1): n represents a randomized region of any length (A, G, C, or T)
  misc_feature (42)..(42): n represents a randomized region of any length (A, G, C, or T)
nccgaccttgtgctttgggagtgctggtccaagggcgttaan

SEQ ID NO: 4 - DNA, 53 bp, Artificial Sequence
  Synthetic
  misc_feature (1)..(1): n represents a randomized region of any length (A, G, C, or T)
  misc_feature (53)..(53): n represents a randomized region of any length (A, G, C, or T)
nacgcagcatttcatcgggtccaaaaggggctgctcgggattgcggatatggn

SEQ ID NO: 5 - DNA, 40 bp, Artificial Sequence
  Synthetic
ccgaccttgtgctttgggagtgctggtccaagggcgttaa

SEQ ID NO: 6 - DNA, 51 bp, Artificial Sequence
  Synthetic
acgcagcatttcatcgggtccaaaaggggctgctcgggattgcggatatgg

SEQ ID NO: 7 - DNA, 60 bp, Artificial Sequence
  Synthetic
  misc_feature (1)..(60): any nucleotide
nnnnnnnnnnnnnnnnnnnnnnnnnnnnnnnnnnnnnnnnnnnnnnnnnnnnnnnnnnnn

SEQ ID NO: 8 - DNA, 33 bp, Artificial Sequence
  Synthetic
  misc_feature (1)..(33): any nucleotide
nnnnnnnnnnnnnnnnnnnnnnnnnnnnnnnnn

SEQ ID NO: 9 - DNA, 34 bp, Artificial Sequence
  Synthetic
  misc_feature (1)..(34): Any nucleotide
nnnnnnnnnnnnnnnnnnnnnnnnnnnnnnnnnn

SEQ ID NO: 10 - DNA, 38 bp, Artificial Sequence
  Synthetic
  misc_feature (1)..(38): Any nucleotide
nnnnnnnnnnnnnnnnnnnnnnnnnnnnnnnnnnnnnn

SEQ ID NO: 11 - DNA, 21 bp, Artificial Sequence
  Synthetic
  misc_feature (1)..(21): Any nucleotide
nnnnnnnnnnnnnnnnnnnnn

SEQ ID NO: 12 - DNA, 19 bp, Artificial Sequence
  Synthetic
  misc_feature (1)..(19): Any nucleotide
nnnnnnnnnnnnnnnnnnn

SEQ ID NO: 13 - DNA, 17 bp, Artificial Sequence
  Synthetic
  misc_feature (1)..(17): Any nucleotide
nnnnnnnnnnnnnnnnn

SEQ ID NO: 14 - DNA, 16 bp, Artificial Sequence
  Synthetic
  misc_feature (1)..(16): Any nucleotide
nnnnnnnnnnnnnnnn

SEQ ID NO: 15 - DNA, 15 bp, Artificial Sequence
  Synthetic
  misc_feature (1)..(15): Any nucleotide
nnnnnnnnnnnnnnn

SEQ ID NO: 16 - DNA, 14 bp, Artificial Sequence
  Synthetic
  misc_feature (1)..(14): Any nucleotide
nnnnnnnnnnnnnn

SEQ ID NO: 17 - DNA, 13 bp, Artificial Sequence
  Synthetic
  misc_feature (1)..(13): Any nucleotide
nnnnnnnnnnnnn

SEQ ID NO: 18 - DNA, 73 bp, Artificial Sequence
  Synthetic
  misc_feature (1)..(16): Any nucleotide
  misc_feature (57)..(73): Any nucleotide
nnnnnnnnnnnnnnnnccgaccttgtgctttgggagtgctggtccaagggcgttaannnnnnnnnnnnnnnnn

SEQ ID NO: 19 - DNA, 89 bp, Artificial Sequence
  Synthetic
  misc_feature (1)..(21): Any nucleotide
  misc_feature (71)..(89): Any nucleotide
nnnnnnnnnnnnnnnnnnnnnacgcagcatttcatcgggtccaaaaggggctgctcgggattgcggatatggnnnnnnnnnnnnnnnnn

SEQ ID NO: 20 - DNA, 123 bp, Artificial Sequence
  Synthetic
  misc_feature (20)..(40): Any nucleotide
  misc_feature (92)..(108): Any nucleotide
ggaacacggttcgagctggnnnnnnnnnnnnnnnnnnnnnacgcagcatttcatcgggtccaaaaggggctgctcgggattgcggatatggnnnnnnnnnnnnnnnnngccgtgcaggtccga

SEQ ID NO: 21 - DNA, 19 bp, Artificial Sequence
  Synthetic
ggaacacggttcgagctgg

SEQ ID NO: 22 - DNA, 15 bp, Artificial Sequence
  Synthetic
tcggacctgcacggc

SEQ ID NO: 23 - DNA, 107 bp, Artificial Sequence
  Synthetic
  misc_feature (20)..(35): Any nucleotide
  misc_feature (76)..(84): n is a, c, g, or t
  misc_feature (85)..(92): Any nucleotide
ggaacacggttcgagctggnnnnnnnnnnnnnnnnccgaccttgtgctttgggagtgctggtccaagggcgttaannnnnnnnnnnnnnnnngccgtgcaggtccga

SEQ ID NO: 24 - DNA, 119 bp, Artificial Sequence
  Synthetic
  misc_feature (20)..(40): Any nucleotide
  misc_feature (92)..(101): n is a, c, g, or t
  misc_feature (102)..(114): Any nucleotide
ggaacacggttcgagctggnnnnnnnnnnnnnnnnnnnnnacgcagcatttcatcgggtccaaaaggggctgctcgggattgcggatatggnnnnnnnnnnnnngccgtgcaggtccga

SEQ ID NO: 25 - DNA, 92 bp, Artificial Sequence
  Synthetic
  misc_feature (20)..(35): Any nucleotide
  misc_feature (76)..(85): n is a, c, g, or t
  misc_feature (86)..(92): Any nucleotide
ggaacacggttcgagctggnnnnnnnnnnnnnnnnccgaccttgtgctttgggagtgctggtccaagggcgttaannnnnnnnnnnnnnnnn

SEQ ID NO: 26 - DNA, 108 bp, Artificial Sequence
  Synthetic
  misc_feature (20)..(40): Any nucleotide
  misc_feature (92)..(108): Any nucleotide
ggaacacggttcgagctggnnnnnnnnnnnnnnnnnnnnnacgcagcatttcatcgggtccaaaaggggctgctcgggattgcggatatggnnnnnnnnnnnnnnnnn

SEQ ID NO: 27 - DNA, 104 bp, Artificial Sequence
  Synthetic
  misc_feature (20)..(40): Any nucleotide
  misc_feature (92)..(104): Any nucleotide
ggaacacggttcgagctggnnnnnnnnnnnnnnnnnnnnnacgcagcatttcatcgggtccaaaaggggctgctcgggattgcggatatggnnnnnnnnnnnnn

SEQ ID NO: 28 - DNA, 99 bp, Artificial Sequence
  Synthetic
  misc_feature (20)..(84): Any nucleotide
ggaacacggttcgagctggnnnnnnnnnnnnnnnnnnnnnnnnnnnnnnnnnnnnnnnnnnnnnnnnnnnnnnnnnnnnnnnnngccgtgcaggtccga

SEQ ID NO: 29 - DNA, 98 bp, Artificial Sequence
  Synthetic
  misc_feature (20)..(83): Any nucleotide
ggaacacggttcgagctggnnnnnnnnnnnnnnnnnnnnnnnnnnnnnnnnnnnnnnnnnnnnnnnnnnnnnnnnnnnnnnnngccgtgcaggtccga

SEQ ID NO: 30 - DNA, 97 bp, Artificial Sequence
  Synthetic
  misc_feature (20)..(82): Any nucleotide
ggaacacggttcgagctggnnnnnnnnnnnnnnnnnnnnnnnnnnnnnnnnnnnnnnnnnnnnnnnnnnnnnnnnnnnnnnngccgtgcaggtccga

SEQ ID NO: 31 - DNA, 96 bp, Artificial Sequence
  Synthetic
  misc_feature (20)..(81): Any nucleotide
ggaacacggttcgagctggnnnnnnnnnnnnnnnnnnnnnnnnnnnnnnnnnnnnnnnnnnnnnnnnnnnnnnnnnnnnnngccgtgcaggtccga

SEQ ID NO: 32 - DNA, 95 bp, Artificial Sequence
  Synthetic
  misc_feature (20)..(80): Any nucleotide
ggaacacggttcgagctggnnnnnnnnnnnnnnnnnnnnnnnnnnnnnnnnnnnnnnnnnnnnnnnnnnnnnnnnnnnnngccgtgcaggtccga

SEQ ID NO: 33 - DNA, 94 bp, Artificial Sequence
  Synthetic
  misc_feature (20)..(79): Any nucleotide
ggaacacggttcgagctggnnnnnnnnnnnnnnnnnnnnnnnnnnnnnnnnnnnnnnnnnnnnnnnnnnnnnnnnnnnngccgtgcaggtccga

SEQ ID NO: 34 - DNA, 93 bp, Artificial Sequence
  Synthetic
  misc_feature (20)..(78): Any nucleotide
ggaacacggttcgagctggnnnnnnnnnnnnnnnnnnnnnnnnnnnnnnnnnnnnnnnnnnnnnnnnnnnnnnnnnnngccgtgcaggtccga

SEQ ID NO: 35 - DNA, 123 bp, Artificial Sequence
  Synthetic
ggaacacggttcgagctggtgcgacatgatacttctgcgaacgcagcatttcatcgggtccaaaaggggctgctcaggattgcggatatggactacgtgctgtgtttcgccgtgcaggtccga

SEQ ID NO: 36 - DNA, 122 bp, Artificial Sequence
  Synthetic
ggaacacggttcgagctggtgcgacatgatacttctgcgaacgcagcatttcatcgggtccaaaggggctgctcaggattgcggatatggactacgtgctgtgtttcgccgtgcaggtccga

SEQ ID NO: 37 - DNA, 122 bp, Artificial Sequence
  Synthetic
ggaacacggttcgagctggtgcgacatgatacttctgcgaacgcagcatttcatcgggtccaaaagggctgctcaggattgcggatatggactacgtgctgtgtttcgccgtgcaggtccga

SEQ ID NO: 38 - DNA, 121 bp, Artificial Sequence
  Synthetic
ggaacacggttcgagctggtgcgacatgatacttctgcgaacgcagcatttcatcgggtccaaagggctgctcaggattgcggatatggactacgtgctgtgtttcgccgtgcaggtccga

SEQ ID NO: 39 - DNA, 123 bp, Artificial Sequence
  Synthetic
ggaacacggttcgagctggtgcgacatgatacttctgcagacgcagcatttcatcgggtccaaaaggggctgctcaggattgcggatatggactacgtgctgtgtttcgccgtgcaggtccga

SEQ ID NO: 40 - DNA, 123 bp, Artificial Sequence
  Synthetic
ggaacacggttcgagctggtactaaacttcgctaaacaacacgcagcatttcatcgggtccaaaaggggctgctcgggattgcggatatggacccgtcagcttatcgtgccgtgcaggtccga

SEQ ID NO: 41 - DNA, 104 bp, Artificial Sequence
  Synthetic
ggaacacggttcgagctggtactaaacttcgctaaacaacacgcagcatttcatcgggtccaaaaggggctgctcgggattgcggatatggacccgtcagctta

SEQ ID NO: 42 - DNA, 100 bp, Artificial Sequence
  Synthetic
cacggttcgagctggtactaaacttcgctaaacaacacgcagcatttcatcgggtccaaaaggggctgctcgggattgcggatatggacccgtcagctta

SEQ ID NO: 43 - DNA, 122 bp, Artificial Sequence
  Synthetic
ggaacacggttcgagctggtactaaacttcgctaaacaacacgcagcatttcatcgggtccaaaggggctgctcgggattgcggatatggacccgtcagcttatcgtgccgtgcaggtccga

SEQ ID NO: 44 - DNA, 122 bp, Artificial Sequence
  Synthetic
ggaacacggttcgagctggtactaaacttcgctaaacaacacgcagcatttcatcgggtccaaaagggctgctcgggattgcggatatggacccgtcagcttatcgtgccgtgcaggtccga

SEQ ID NO: 45 - DNA, 124 bp, Artificial Sequence
  Synthetic
ggaacacggttcgagctggtactaaacttcgctaaacaacacgcagcatttcatcgggtccaaaaaggggctgctcgggattgcggatatggacccgtcagcttatcgtgccgtgcaggtccga

SEQ ID NO: 46 - DNA, 121 bp, Artificial Sequence
  Synthetic
ggaacacggttcgagctggtactaaacttcgctaaacaacacgcagcatttcatcgggtccaaagggctgctcgggattgcggatatggacccgtcagcttatcgtgccgtgcaggtccga

SEQ ID NO: 47 - DNA, 94 bp, Artificial Sequence
  Synthetic
ggaacacggttcgagctggtctgctctaggattgcggtgtctcgtatctatggttagttccacgtctgttgctttgtgtgccgtgcaggtccga

SEQ ID NO: 48 - DNA, 107 bp, Artificial Sequence
  Synthetic
ggaacacggttcgagctggccaagcgagagatataccgaccttgtgctttgggagtgctggtccaagggcgttaatactgccagtttggtgtgccgtgcaggtccga

SEQ ID NO: 49 - DNA, 107 bp, Artificial Sequence
  Synthetic
ggaacacggttcgagctggccttcgtgatctgtccccgaccttgtgctttgggagtgctggtccaagggcgttaacaatgtgagtttatgttgccgtgcaggtccga

SEQ ID NO: 50 - DNA, 123 bp, Artificial Sequence
  Synthetic
ggaacacggttcgagctggaacgtcaccgtctcgtatcatacgcagcatttcatcgggtccaaaaggggctgctcgggattgcggatatggacccgatccttctgtgcgccgtgcaggtccga

SEQ ID NO: 51 - DNA, 107 bp, Artificial Sequence
  Synthetic
ggaacacggttcgagctggaaaatcactatgcgaaccgaccttgtgctttgggagtgctggtccaagggcgttaagtctactgcttcacatcgccgtgcaggtccga

SEQ ID NO: 52 - DNA, 107 bp, Artificial Sequence
  Synthetic
ggaacacggttcgagctgggatgcatattctgtttccgaccttgtgctttgggagtgctggtccaaggtcgttaatggtttgttctttccgtgccgtgcaggtccga

SEQ ID NO: 53 - DNA, 107 bp, Artificial Sequence
  Synthetic
ggaacacggttcgagctggtatgccattccaataaccgaccttgtgctttgggagtgctggtccaagggcgttaatttattactgttgtgttgccgtgcaggtccga

SEQ ID NO: 54 - DNA, 93 bp, Artificial Sequence
  Synthetic
ggaacacggttcgagctggagacctaattagggattgccttccggtctgcgggtgatgtctggttgacagtttggtttgccgtgcaggtccga

SEQ ID NO: 55 - DNA, 93 bp, Artificial Sequence
  Synthetic
ggaacacggttcgagctggtctgctctaagttgcggtgtctcgtatctatggttagttccacgtctgttgctttgtgtgccgtgcaggtccga

SEQ ID NO: 56 - DNA, 99 bp, Artificial Sequence
  Synthetic
ggaacacggttcgagctggacattggactggttctacgcttggtggtgtcttcctctatgttcttccgttgtttatcccgccgtgccgtgcaggtccga

SEQ ID NO: 57 - DNA, 97 bp, Artificial Sequence
  Synthetic
ggaacacggttcgagctggtctgctctaggattgcggtgtctcgtatctatggttagttccacgtctgttgctttgtgccgtgccgtgcaggtccga

SEQ ID NO: 58 - DNA, 99 bp, Artificial Sequence
  Synthetic
ggaacacggttcgagctggtacgcgctttgtacacaggattgctttggaagtgctggttatcatgtgatttcttgtcgtgccgtgccgtgcaggtccga

SEQ ID NO: 59 - DNA, 99 bp, Artificial Sequence
  Synthetic
ggaacacggttcgagctggtgccgcgtcaacctcctggttctattccaaactgctttgggagtgctggtcgtttggtttgccgtgccgtgcaggtccga

SEQ ID NO: 60 - DNA, 99 bp, Artificial Sequence
  Synthetic
ggaacacggttcgagctggcacactcgcgcgattggctttcggtctgggaaaatagtgttgtctggttcaccgtattgtgccgtgccgtgcaggtccga

SEQ ID NO: 61 - DNA, 119 bp, Artificial Sequence
  Synthetic
ggaacacggttcgagctggttctatactgctaatacgtacgcagtcatgtccaaaaggggctgctcgggattgcggatatggatcgtgactgttagtgtgccgtgccgtgcaggtccga

SEQ ID NO: 62 - DNA, 119 bp, Artificial Sequence
  Synthetic
ggaacacggttcgagctggtatccaactcgtcagttcaacacgcagcattccaaaaggggctgctcgggattgcggatatggttgctgcagtttggttggccgtgccgtgcaggtccga

SEQ ID NO: 63 - DNA, 115 bp, Artificial Sequence
  Synthetic
ggaacacggttcgagctggcaatgacttttaattgaacagacgcattcatcaggggctgctcgggattgcggatatggaatgctgcttgttttgcgccgtgccgtgcaggtccga

SEQ ID NO: 64 - DNA, 128 bp, Artificial Sequence
  Synthetic
ggaacacggttcgagctggtggaacggagcgcgaattctaacgcagcatttcatcgggtccaaaagggactgctcgggattgcggatatgggagtcgatgtttgtcgcgccgtgccgtgcaggtccga

SEQ ID NO: 65 - DNA, 112 bp, Artificial Sequence
  Synthetic
ggaacacggttcgagctggaccaaaatagttggatccgaccttgtgctttgggagtgctggtccaagggcgttaatacatacagtttagtgtgccgtgccgtgcaggtccga

SEQ ID NO: 66 - DNA, 127 bp, Artificial Sequence
  Synthetic
ggaacacggttcgagctgggacgcagtatccacatgtctaacgcagcatttcatcgggtccaaaagggctgctcgggattgcggatatggacacgactgttaatgttgccgtgccgtgcaggtccga

SEQ ID NO: 67 - DNA, 112 bp, Artificial Sequence
  Synthetic
ggaacacggttcgagctggtactgctatctttgtaccgaccttgtgctttgggagtgctggtccaagggcgttaagatcgatccttcgctgtgccgtgccgtgcaggtccga

SEQ ID NO: 68 - DNA, 126 bp, Artificial Sequence
  Synthetic
ggaacacggttcgagctggccactagtactcaacgtagaaacgcagcatttcatcgggtccaaagggctgctcgggattgcggatatggacccgttgctttatgttgccgtgccgtgcaggtccga

SEQ ID NO: 69 - DNA, 112 bp, Artificial Sequence
  Synthetic
ggaacacggttcgagctggttatatgcgaatacttccgaccttgtgctttgggagtgctggtccaagggcgttaagcgtgactctttatgttgccgtgccgtgcaggtccga

SEQ ID NO: 70 - DNA, 107 bp, Artificial Sequence
  Synthetic
ggaacacggttcgagctgggttctttgctcgtagaccgaccttgtgctttgggagtgctggtccaagggcgttaaaatttgcagctgatgttgccgtgcaggtccga

SEQ ID NO: 71 - DNA, 107 bp, Artificial Sequence
  Synthetic
ggaacacggttcgagctgggtgccacgttttactgccgaccttgtgctttgggagtgctggtccaagggcgttaagcatgtctgttgtctgtgccgtgcaggtccga

SEQ ID NO: 72 - DNA, 123 bp, Artificial Sequence
  Synthetic
ggaacacggttcgagctggtgacgggggcaacagccattgacgcagcatttcatcgggtccaaaaggggctgctcaggattgcggatatggacccgagactttacgttgccgtgcaggtccga

SEQ ID NO: 73 - DNA, 123 bp, Artificial Sequence; identical to SEQ ID NO: 72
  Synthetic

SEQ ID NO: 74 - DNA, 107 bp, Artificial Sequence
  Synthetic
ggaacacggttcgagctggttagtatcgtgagcttccgaccttgtgctttgggagtgctggtccaagggcgttaagttacttcctttatgttgccgtgcaggtccga

SEQ ID NO: 75 - DNA, 107 bp, Artificial Sequence
  Synthetic
ggaacacggttcgagctggccaacacccgattacaccgaccttgtgctttgggagtgctggtccaagggcgttaatcattactgctatacgtgccgtgcaggtccga

SEQ ID NO: 76 - DNA, 107 bp, Artificial Sequence
  Synthetic
ggaacacggttcgagctggacatcaataaaaacatccgaccttgtgctttgggagtgctggtccaagggcgttaacaatgctacctgagtttgccgtgcaggtccga

SEQ ID NO: 77 - DNA, 107 bp, Artificial Sequence
  Synthetic
ggaacacggttcgagctggttatatgcgaatacttccgaccttgtgctttgggagtgctggtccaagggcgttaagcgtgactctttatgttgccgtgcaggtccga

SEQ ID NO: 78 - DNA, 107 bp, Artificial Sequence
  Synthetic
ggaacacggttcgagctggtttccgattggtctgaccgaccttgtgctttgggagtgctggtccaagggcgttaacttgtccactttacgttgccgtgcaggtccga

SEQ ID NO: 79 - DNA, 107 bp, Artificial Sequence
  Synthetic
ggaacacggttcgagctggaaaacgtagaaaattaccgaccttgtgctttgggagtgctggtccaagggcgttaagcctaaactgctagcgtgccgtgcaggtccga

SEQ ID NO: 80 - DNA, 123 bp, Artificial Sequence
  Synthetic
ggaacacggttcgagctggccactagtactcaacgtagaaacgcagcatttcatcgggtccaaaaggggctgctcgggattgcggatatggacccgttgctttatgttgccgtgcaggtccga

SEQ ID NO: 81 - DNA, 107 bp, Artificial Sequence
  Synthetic
ggaacacggttcgagctggcacaattgactgcttcccgaccttgtgctttgggagtgctggtccaagggcgttaatccggtcagtttggctcgccgtgcaggtccga

SEQ ID NO: 82 - DNA, 106 bp, Artificial Sequence
  Synthetic
ggaacacggttcgagctggccaagcgagagatataccgaccttgtgctttgggagtgctggtcaagggcgttaatactgccagtttggtgtgccgtgcaggtccga

SEQ ID NO: 83 - DNA, 107 bp, Artificial Sequence
  Synthetic
ggaacacggttcgagctggtactgctatctttgtaccgaccttgtgctttgggagtgctggtccaagggcgttaagatcgatccttcgctgtgccgtgcaggtccga

SEQ ID NO: 84 - DNA, 99 bp, Artificial Sequence; identical to SEQ ID NO: 60
  Synthetic

SEQ ID NO: 85 - DNA, 122 bp, Artificial Sequence
  Synthetic
ggaacacggttcgagctggtactacttcgctaaacaacacgcagcatttcatcgggtccaaaaaggggctgctcgggattgcggatatggacccgtcagcttatcgtgccgtgcaggtccga

SEQ ID NO: 86 - DNA, 94 bp, Artificial Sequence
  Synthetic
ggaacacggttcgagctggaaatcattctactccatggggctgctcgggattgcggatatggagtggtgagtttatgttgccgtgcaggtccga

SEQ ID NO: 87 - DNA, 95 bp, Artificial Sequence
  Synthetic
ggaacacggttcgagctggtctgctctagaagttgcggtgtctcgtatctatggttagttccacgtctgttgctttgtgtgccgtgcaggtccga

SEQ ID NO: 88 - DNA, 15 bp, Artificial Sequence
  Synthetic
cacggttcgagctgg

SEQ ID NO: 89 - DNA, 89 bp, Artificial Sequence
  Synthetic
ctggtactaaacttcgctaaacaacacgcagcatttcatcgggtccaaaaggggctgctcgggattgcggatatggacccgtcagctta

SEQ ID NO: 90 - DNA, 73 bp, Artificial Sequence
  Synthetic
ctaaacaacacgcagcatttcatcgggtccaaaaggggctgctcgggattgcggatatggacccgtcagctta

SEQ ID NO: 91 - DNA, 123 bp, Artificial Sequence; identical to SEQ ID NO: 40
  Synthetic

SEQ ID NO: 92 - DNA, 123 bp, Artificial Sequence; identical to SEQ ID NO: 40
  Synthetic

SEQ ID NO: 93 - DNA, 104 bp, Artificial Sequence; identical to SEQ ID NO: 41
  Synthetic

SEQ ID NO: 94 - DNA, 104 bp, Artificial Sequence; identical to SEQ ID NO: 41
  Synthetic

SEQ ID NO: 95 - DNA, 100 bp, Artificial Sequence; identical to SEQ ID NO: 42
  Synthetic

SEQ ID NO: 96 - DNA, 100 bp, Artificial Sequence; identical to SEQ ID NO: 42
  Synthetic

SEQ ID NO: 97 - DNA, 89 bp, Artificial Sequence; identical to SEQ ID NO: 89
  Synthetic

SEQ ID NO: 98 - DNA, 89 bp, Artificial Sequence; identical to SEQ ID NO: 89
  Synthetic

SEQ ID NO: 99 - DNA, 73 bp, Artificial Sequence; identical to SEQ ID NO: 90
  Synthetic

SEQ ID NO: 100 - DNA, 73 bp, Artificial Sequence; identical to SEQ ID NO: 90
  Synthetic

SEQ ID NO: 101 - DNA, 22 bp, Artificial Sequence
  Synthetic
cacggttcgagctggtactaaa

SEQ ID NO: 102 - DNA, 21 bp, Artificial Sequence
  Synthetic
taagctgacgggtccatatcc

SEQ ID NO: 103 - DNA, 20 bp, Artificial Sequence
  Synthetic
acggttcgagctggtactaa

SEQ ID NO: 104 - DNA, 20 bp, Artificial Sequence
  Synthetic
atatccgcaatcccgagcag

SEQ ID NO: 105 - DNA, 24 bp, Artificial Sequence
  Synthetic
acacgcagcatttcatcgggtcca